(19) United States
(12) Patent Application Publication
(10) Pub. No.: US 2025/0319185 A1
(43) Pub. Date: Oct. 16, 2025

(54) BCMA-DIRECTED CELLULAR IMMUNOTHERAPY COMPOSITIONS AND METHODS

(71) Applicant: NKARTA, INC., South San Francisco, CA (US)

(72) Inventors: Kanya Lakshmi RAJANGAM, San Francisco, CA (US); James Barnaby TRAGER, Albany, CA (US); Luxuan GUO BUREN, San Francisco, CA (US); Chao GUO, San Francisco, CA (US); Alexandra Leida Liana LAZETIC, San Jose, CA (US); Nitinkumar Lakshmanbhai PATEL, Fremont, CA (US)

(21) Appl. No.: 18/578,164

(22) PCT Filed: Jul. 8, 2022
(86) PCT No.: PCT/US2022/073567

§ 371 Date: Jan. 10, 2024

Related U.S. Application Data
(60) Provisional application No. 63/220,842, filed on Jul. 12, 2021.

(51) Int. Cl. A61K 40/42; A61K 40/11; A61K 40/15; A61K 40/31; C07K 14/725; C07K 16/28

(52) U.S. Cl. CPC A61K 40/4215; A61K 40/11; A61K 40/15; A61K 40/31; C07K 14/7051; C07K 16/2878; C07K 2317/569

(57) ABSTRACT

Provided for herein in several embodiments are anti-BCMA binding moieties. These anti-BCMA binding moieties may be used in BCMA-directed chimeric antigen receptors (CARs). Also disclosed herein are immune cell-based compositions comprising the anti-BCMA binding moieties and BCMA-directed CARs. In several embodiments, the immune-cell based compositions also target an additional tumor marker and/or an additional epitope of BCMA. In several embodiments, the BCMA-directed CAR is expressed in a Natural Killer cell. In several embodiments, combinations of BCMA-CAR-expressing NK cells are administered in conjunction with, for example CAR-expressing NK cells and/or CAR-expressing T cells that are directed to an additional cancer marker and/or an additional epitope of BCMA. Also provided for herein are methods and uses of the chimeric antigen receptors in immunotherapy.

### CROSS REFERENCE TO RELATED APPLICATIONS

This application claims priority to U.S. Provisional Patent Application No. 63/220,842, filed Jul. 12, 2021, the entire contents of which is incorporated by reference herein.

### INCORPORATION BY REFERENCE OF MATERIAL IN ASCII TEXT FILE

A Sequence Listing contained in the following ASCII text file being submitted concurrently herewith: File Name: NKT074PR_ST26.xml; created Jul. 8, 2022, which is 13,071,224 bytes in size. This Sequence Listing in electronic format is hereby expressly incorporated by reference in its entirety.

### FIELD

Some embodiments of the methods and compositions provided herein relate to cellular therapy employing B-Cell Maturation Antigen (BCMA)-targeting chimeric antigen receptors (CARs). Some embodiments relate to one or more of such constructs expressed by NK and/or T cells. Also disclosed herein are antigen binding molecules that bind to BCMA.

### BACKGROUND

As further knowledge is gained about various cancers and characteristics of a cancerous cell that specifically distinguish that cell from a healthy cell, therapeutics are under development that leverage the distinct features of a cancerous cell. Immunotherapies that employ engineered immune cells are one approach to treating cancers.

### SUMMARY

Immunotherapy presents a new technological advancement in the treatment of disease, where immune cells are engineered to express certain targeting and/or effector molecules that specifically identify and react to diseased or damaged cells. This represents a promising advance due, at least in part, to the potential for specifically targeting diseased or damaged cells, as opposed to more traditional approaches, such as chemotherapy, where all cells are impacted, and the desired outcome is that sufficient healthy cells survive to mitigate side effects in the patient. One immunotherapy approach is the recombinant expression of chimeric antigen receptors (CARs) in immune cells to achieve the targeted recognition and destruction of aberrant cells of interest, such as cancer.

In certain cancers, patient responses to immunotherapy are initially robust and positive, but are short-lived. Such profiles are addressed by several embodiments of the cellular immunotherapy compositions provided for herein. For example, in several embodiments, natural killer (NK) cells are engineered to express one or more chimeric antigen receptors (CARs). Due to the enhanced cytotoxicity that the engineered NK cells exhibit, in conjunction with the inherent rapid immune response of NK cells, several embodiments allow for an enhanced initial anti-cancer effect that can substantially reduce, or even eliminate, tumor burden. In several embodiments, such engineered NK cells are particularly important, at least in part due to their reduced immunogenic potential as compared to T cells, because aggressive cancers may not allow enough time for an autologous T cell therapy to be generated.

An anti-BCMA binding moiety comprising a heavy chain variable region (VH) comprising a HCDR1, HCDR2, and HCDR3, wherein: the HCDR1 comprises a sequence having at least 85%, 90%, 91%, 92%, 93%, 94%, 95%, 96%, 97%, 98%, 99%, or 100% identity to any one of SEQ ID NOs: 114, 105, 107, 129, 104, 106, 108-113, or 115-128, the HCDR2 comprises a sequence having at least 85%, 90%, 91%, 92%, 93%, 94%, 95%, 96%, 97%, 98%, 99%, or 100% identity to any one of SEQ ID NOs: 140, 131, 133, 155, 130, 132, 134-139, or 141-154, and the HCDR3 comprises a sequence having at least 85%, 90%, 91%, 92%, 93%, 94%, 95%, 96%, 97%, 98%, 99%, or 100% identity to any one of SEQ ID NOs: 166, 157, 159, 181, 156, 158, 160-165, or 167-180. In several embodiments, the anti-BMCA binding moiety further comprises a light chain variable region (VL) comprising an LCDR1, LCDR2, and LCDR3, wherein the LCDR1 comprises a sequence having at least 85%, 90%, 91%, 92%, 93%, 94%, 95%, 96%, 97%, 98%, 99%, or 100% identity to any one of SEQ ID NOs: 395, 386, 388, 410, 385, 387, 389-392, or 396-409, the LCDR2 comprises a sequence having at least 85%, 90%, 91%, 92%, 93%, 94%, 95%, 96%, 97%, 98%, 99%, or 100% identity to any one of SEQ ID NOs: 421, 412 414, 436, 411, 413, 415-420, or 422-435, and the LCDR3 comprises a sequence having at least 85%, 90%, 91%, 92%, 93%, 94%, 95%, 96%, 97%, 98%, 99%, or 100% identity to any one of SEQ ID NOs: 447, 438, 440, 462, 437, 439, 441-446, or 448-446. In several embodiments, provided for herein is a BCMA-directed chimeric antigen receptor (CAR) comprising the anti-BCMA binding moiety. Also provided is a population of immune cells engineered to express the anti-BMCA binding moiety, optionally in the form of the BCMA-directed CAR. In several embodiments, provided for herein are methods of treating cancer, such as multiple myeloma, comprising administering to a subject a population of immune cells (e.g., natural killer (NK) cells and/or T cells) engineered to express the BMCA-directed. Uses of the BCMA-directed CAR in the manufacture of a medicament and/or for the treatment of cancer is also provided for herein, in several embodiments.

In several embodiments, the HCDR1 of the anti-BCMA binding moiety comprises a sequence having at least 85%, 90%, 91%, 92%, 93%, 94%, 95%, 96%, 97%, 98%, 99%, or 100% identity to SEQ ID NO: 114, the HCDR2 comprises a sequence having at least 85%, 90%, 91%, 92%, 93%, 94%, 95%, 96%, 97%, 98%, 99%, or 100% identity to SEQ ID NO: 140, and the HCDR3 comprises a sequence having at least 85%, 90%, 91%, 92%, 93%, 94%, 95%, 96%, 97%, 98%, 99%, or 100% identity to SEQ ID NO: 166.

In some embodiments, the HCDR1 of the anti-BCMA binding moiety comprises a sequence having at least 85%, 90%, 91%, 92%, 93%, 94%, 95%, 96%, 97%, 98%, 99%, or 100% identity to SEQ ID NO: 105, the HCDR2 comprises a sequence having at least 85%, 90%, 91%, 92%, 93%, 94%, 95%, 96%, 97%, 98%, 99%, or 100% identity to SEQ ID NO: 131, and the HCDR3 comprises a sequence having at least 85%, 90%, 91%, 92%, 93%, 94%, 95%, 96%, 97%, 98%, 99%, or 100% identity to SEQ ID NO: 157.

In some embodiments, the HCDR1 of the anti-BCMA binding moiety comprises a sequence having at least 85%, 90%, 91%, 92%, 93%, 94%, 95%, 96%, 97%, 98%, 99%, or 100% identity to SEQ ID NO: 107, the HCDR2 comprises a sequence having at least 85%, 90%, 91%, 92%, 93%, 94%, 95%, 96%, 97%, 98%, 99%, or 100% identity to SEQ ID NO: 133, and the HCDR3 comprises a sequence having at least 85%, 90%, 91%, 92%, 93%, 94%, 95%, 96%, 97%, 98%, 99%, or 100% identity to SEQ ID NO: 159.

In some embodiments, the HCDR1 of the anti-BCMA binding moiety comprises a sequence having at least 85%, 90%, 91%, 92%, 93%, 94%, 95%, 96%, 97%, 98%, 99%, or 100% identity to SEQ ID NO: 129, the HCDR2 comprises a sequence having at least 85%, 90%, 91%, 92%, 93%, 94%, 95%, 96%, 97%, 98%, 99%, or 100% identity to SEQ ID NO: 155, and the HCDR3 comprises a sequence having at least 85%, 90%, 91%, 92%, 93%, 94%, 95%, 96%, 97%, 98%, 99%, or 100% identity to SEQ ID NO: 181.

In several embodiments, the LCDR1 of the anti-BCMA binding moiety comprises a sequence having at least 85%, 90%, 91%, 92%, 93%, 94%, 95%, 96%, 97%, 98%, 99%, or 100% identity to SEQ ID NO: 395, the LCDR2 comprises a sequence having at least 85%, 90%, 91%, 92%, 93%, 94%, 95%, 96%, 97%, 98%, 99%, or 100% identity to SEQ ID NO: 421, and the LCDR3 comprises a sequence having at least 85%, 90%, 91%, 92%, 93%, 94%, 95%, 96%, 97%, 98%, 99%, or 100% identity to SEQ ID NO: 447.

In some embodiments, the LCDR1 of the anti-BCMA binding moiety comprises a sequence having at least 85%, 90%, 91%, 92%, 93%, 94%, 95%, 96%, 97%, 98%, 99%, or 100% identity to SEQ ID NO: 386, the LCDR2 comprises a sequence having at least 85%, 90%, 91%, 92%, 93%, 94%, 95%, 96%, 97%, 98%, 99%, or 100% identity to SEQ ID NO: 412, and the LCDR3 comprises a sequence having at least 85%, 90%, 91%, 92%, 93%, 94%, 95%, 96%, 97%, 98%, 99%, or 100% identity to SEQ ID NO: 438.

In some embodiments, the LCDR1 of the anti-BCMA binding moiety comprises a sequence having at least 85%, 90%, 91%, 92%, 93%, 94%, 95%, 96%, 97%, 98%, 99%, or 100% identity to SEQ ID NO: 388, the LCDR2 comprises a sequence having at least 85%, 90%, 91%, 92%, 93%, 94%, 95%, 96%, 97%, 98%, 99%, or 100% identity to SEQ ID NO: 414, and the LCDR3 comprises a sequence having at least 85%, 90%, 91%, 92%, 93%, 94%, 95%, 96%, 97%, 98%, 99%, or 100% identity to SEQ ID NO: 440.

In some embodiments, the LCDR1 of the anti-BCMA binding moiety comprises a sequence having at least 85%, 90%, 91%, 92%, 93%, 94%, 95%, 96%, 97%, 98%, 99%, or 100% identity to SEQ ID NO: 410, the LCDR2 comprises a sequence having at least 85%, 90%, 91%, 92%, 93%, 94%, 95%, 96%, 97%, 98%, 99%, or 100% identity to SEQ ID NO: 436, and the LCDR3 comprises a sequence having at least 85%, 90%, 91%, 92%, 93%, 94%, 95%, 96%, 97%, 98%, 99%, or 100% identity to SEQ ID NO: 462.

In several embodiments, the VH of the anti-BCMA binding moiety comprises a sequence having at least 85%, 90%, 91%, 92%, 93%, 94%, 95%, 96%, 97%, 98%, 99%, or 100% identity to any one of SEQ ID NOs: 270, 261, 263, 285, 260, 262, 263-269, or 271-284. In several embodiments, the VL of the anti-BCMA binding moiety comprises a sequence having at least 85%, 90%, 91%, 92%, 93%, 94%, 95%, 96%, 97%, 98%, 99%, or 100% identity to any one of SEQ ID NOs: 551, 542, 544, 566, 541, 543, 545-550, or 552-565.

In several embodiments, the HCDR1 of the anti-BCMA binding moiety is encoded by a nucleic acid comprising a sequence having at least 85%, 90%, 91%, 92%, 93%, 94%, 95%, 96%, 97%, 98%, 99%, or 100% identity to any one of SEQ ID NOs: 192, 183, 185, 207, 182, 184, 186-191, or 192-206, the HCDR2 of the anti-BCMA binding moiety is encoded by a nucleic acid comprising a sequence having at least 85%, 90%, 91%, 92%, 93%, 94%, 95%, 96%, 97%, 98%, 99%, or 100% identity to any one of SEQ ID NOs: 218, 209, 211, 233, 208, 210, 212-217, or 219-232; and the HCDR3 of the anti-BCMA binding moiety is encoded by a nucleic acid comprising a sequence having at least 85%, 90%, 91%, 92%, 93%, 94%, 95%, 96%, 97%, 98%, 99%, or 100% identity to any one of SEQ ID NOs: 244, 235, 237, 259, 234, 236, 238-243, or 245-258.

In several embodiments, the LCDR1 of the anti-BCMA binding moiety is encoded by a nucleic acid comprising a sequence having at least 85%, 90%, 91%, 92%, 93%, 94%, 95%, 96%, 97%, 98%, 99%, or 100% identity to any one of SEQ ID NOs: 473, 464, 466, 488, 463, 465, 467-472, or 474-487, the LCDR2 of the anti-BCMA binding moiety is encoded by a nucleic acid comprising a sequence having at least 85%, 90%, 91%, 92%, 93%, 94%, 95%, 96%, 97%, 98%, 99%, or 100% identity to any one of SEQ ID NOs: 499, 490, 492, 514, 489, 491, 493-498, or 500-513; and the LCDR3 of the anti-BCMA binding moiety is encoded by a nucleic acid comprising a sequence having at least 85%, 90%, 91%, 92%, 93%, 94%, 95%, 96%, 97%, 98%, 99%, or 100% identity to any one of SEQ ID NOs: 525, 516, 518, 540, 515, 517, 519-524, or 526-539.

In several embodiments, the VH of the anti-BCMA binding moiety is encoded by a nucleic acid comprising a sequence having at least 85%, 90%, 91%, 92%, 93%, 94%, 95%, 96%, 97%, 98%, 99%, or 100% identity to any one of SEQ ID NOs: 296, 287, 289, 311, 286, 288, 290-295, or 297-310. In several embodiments, the VL of the anti-BCMA binding moiety is encoded by a nucleic acid comprising a sequence having at least 85%, 90%, 91%, 92%, 93%, 94%, 95%, 96%, 97%, 98%, 99%, or 100% identity to any one of SEQ ID NOs: 577, 568, 570, 592, 567, 569, 571-576, or 578-591.

In several embodiments, the VH and VL (when present) are separated by a linker. In several embodiments, the linker of the anti-BCMA binding moiety comprises a sequence at least 85%, 90%, 91%, 92,%, 93%, 94%, 95%, 96%, 97%, 98%, 99%, or 100% identity to SEQ ID NO: 1388. In several embodiments, the VH is N-terminal of the VL. In several embodiments, the anti-BCMA binding moiety comprises a sequence having at least 85%, 90%, 91%, 92%, 93%, 94%, 95%, 96%, 97%, 98%, 99%, or 100% identity to any one of SEQ ID NOs: 603, 594, 596, 618, 593, 595, 597-602, or 604-617. In several embodiments, the anti-BCMA binding moiety is encoded by a nucleic acid comprising a sequence having at least 85%, 90%, 91%, 92%, 93%, 94%, 95%, 96%, 97%, 98%, 99%, or 100% identity to any one of SEQ ID NOs: 629, 620, 622, 644, 619, 621, 623-628, 630-643, or 645-670.

In several embodiments, the VL (when present) is N-terminal of the VH. In several embodiments, the anti-BCMA binding moiety comprises a sequence having at least 85%, 90%, 91%, 92%, 93%, 94%, 95%, 96%, 97%, 98%, 99%, or 100% identity to any one of SEQ ID NOs: 681, 672, 674, 696, 671, 673, 675,-680, or 682-695. In several embodiments, the anti-BCMA binding moiety is encoded by a nucleic acid comprising a sequence having at least 85%, 90%, 91%, 92%, 93%, 94%, 95%, 96%, 97%, 98%, 99%, or 100% identity to any one of SEQ ID NOs: 707, 698, 700, 722, 697, 699, 701-706, 708-721, or 723-748.

In several embodiments, the linker of the anti-BCMA binding moiety comprises a sequence having at least 85%, 90%, 91%, 92%, 93%, 94%, 95%, 96%, 97%, 98%, 99%, or 100% identity to of SEQ ID NO: 2260. In several embodiments, the anti-BCMA binding moiety comprises a sequence having at least 85%, 90%, 91%, 92%, 93%, 94%, 95%, 96%, 97%, 98%, 99%, or 100% identity to any one of SEQ ID NOs: 1483, 1474, 1476, 1498, 1473, 1475, 1477-1482, or 1484-1497. In several embodiments, the anti-BCMA binding moiety is encoded by a nucleic acid comprising a sequence having at least 85%, 90%, 91%, 92%, 93%, 94%, 95%, 96%, 97%, 98%, 99%, or 100% identity to any one of SEQ ID NOs: 1509, 1500, 1502, 1524, 1499, 1501, 1503-1508, or 1510-1523.

Also provided for herein is a BCMA-directed chimeric antigen receptor (CAR) comprising an anti-BCMA binding moiety described herein. Also provided for herein is an immune cell comprising an anti-BCMA binding moiety described herein. In several embodiments, the immune cell is a natural killer (NK) cell or T cell.

In several embodiments, the CAR further comprises a hinge domain; a transmembrane domain; and an intracellular signaling domain comprising a CD3ζ subdomain. In several embodiments, the intracellular signaling domain of the BCMA-directed CAR further comprises an OX40 subdomain. In several embodiments, the OX40 subdomain comprises a sequence having at least 85%, 90%, 91%, 92%, 93%, 94%, 95%, 96%, 97%, 98%, 99%, or 100% identity to SEQ ID NO: 1394. In several embodiments, the CD3ζ subdomain comprises a sequence having at least 85%, 90%, 91%, 92%, 93%, 94%, 95%, 96%, 97%, 98%, 99%, or 100% identity to SEQ ID NO: 1395. In several embodiments, the transmembrane domain is a CD8 transmembrane domain. In several embodiments, the CD8 transmembrane domain comprises a sequence having at least 85%, 90%, 91%, 92%, 93%, 94%, 95%, 96%, 97%, 98%, 99%, or 100% identity to SEQ ID NO: 1392. In several embodiments, the hinge domain is a CD8 hinge domain. In several embodiments, the CD8 hinge domain comprises a sequence having at least 85%, 90%, 91%, 92%, 93%, 94%, 95%, 96%, 97%, 98%, 99%, or 100% identity to SEQ ID NO: 1389. In several embodiments, CD28-derived hinge, transmembrane, and/or intracellular domains may be used in place of one or more of the CD8 domains.

In several embodiments, the BCMA-directed CAR comprises a sequence having at least 85%, 90%, 91%, 92%, 93%, 94%, 95%, 96%, 97%, 98%, 99%, or 100% identity to any one of SEQ ID NOs: 3897, 3888, 3890, 3912, 3887, 3889, 3891-3896, or 3898-3911. In several embodiments, the BCMA-directed CAR is encoded by a nucleic acid comprising a sequence having at least 85%, 90%, 91%, 92%, 93%, 94%, 95%, 96%, 97%, 98%, 99%, or 100% identity to any one of SEQ ID NOs: 3923, 3914, 3916, 3938, 3913, 3915, 3917-3922, 3924-3937, or 3939-3964. In several embodiments, the nucleic acid encoding the BCMA-directed CAR further encodes a membrane-bound interleukin 15 (mbIL15) and comprises a sequence having at least 85%, 90%, 91%, 92%, 93%, 94%, 95%, 96%, 97%, 98%, 99%, or 100% identity to any one of SEQ ID NOs: 4001, 3992, 3994, 4016, 3991, 3993, 3995-4000, 4002-4015, or 4017-4042.

In several embodiments, the BCMA-directed CAR comprises a sequence having at least 85%, 90%, 91%, 92%, 93%, 94%, 95%, 96%, 97%, 98%, 99%, or 100% identity to any one of SEQ ID NOs: 4053, 4044, 4046, 4068, 4043, 4045, 4047-4052, or 4054-4067. In several embodiments, the BCMA-directed CAR is encoded by a nucleic acid comprising a sequence having at least 85%, 90%, 91%, 92%, 93%, 94%, 95%, 96%, 97%, 98%, 99%, or 100% identity to any one of SEQ ID NOs: 4079, 4070, 4072, 4094, 4069, 4071, 4073-4078, 4080-4093, or 4095-4120. In several embodiments, the nucleic acid encoding the BCMA-directed CAR further encodes a membrane-bound interleukin 15 (mbIL15) and comprises a sequence having at least 85%, 90%, 91%, 92%, 93%, 94%, 95%, 96%, 97%, 98%, 99%, or 100% identity to any one of SEQ ID NOs: 4157, 4148, 4150, 4172, 4147, 4149, 4151-4156, 4158-4171, or 4173-4198.

In several embodiments, the BCMA-directed CAR comprises a sequence having at least 85%, 90%, 91%, 92%, 93%, 94%, 95%, 96%, 97%, 98%, 99%, or 100% identity to any one of SEQ ID NOs: 3869, 3867, 3868, or 3870. In several embodiments, the BCMA-directed CAR is encoded by a nucleic acid comprising a sequence having at least 85%, 90%, 91%, 92%, 93%, 94%, 95%, 96%, 97%, 98%, 99%, or 100% identity to any one of SEQ ID NOs: 3885, 3883, 3884, or 3886. In several embodiments, the nucleic acid encoding the BCMA-directed CAR further encodes a membrane-bound interleukin 15 (mbIL15) and comprises a sequence having at least 85%, 90%, 91%, 92%, 93%, 94%, 95%, 96%, 97%, 98%, 99%, or 100% identity to any one of SEQ ID NOs: 3837, 3835, 3836, or 3839.

Also provided for herein is a VHH-BCMA-directed chimeric antigen receptor (CAR), the CAR comprising an extracellular anti-BCMA binding moiety, wherein the anti-BCMA binding moiety comprise a heavy chain variable region (VH) comprising a HCDR1, HCDR2, and HCDR3, wherein: the HCDR1 comprises a sequence having at least 85%, 90%, 91%, 92%, 93%, 94%, 95%, 96%, 97%, 98%, 99%, or 100% identity to any one of SEQ ID NOs: 104-129, 1525-1543, or 3117-3139, the HCDR2 comprises a sequence having at least 85%, 90%, 91%, 92%, 93%, 94%, 95%, 96%, 97%, 98%, 99%, or 100% identity to any one of SEQ ID NOs: 130-155, 1544-1562, or 3140-3162; and the HCDR3 comprises a sequence having at least 85%, 90%, 91%, 92%, 93%, 94%, 95%, 96%, 97%, 98%, 99%, or 100% identity to any one of SEQ ID NOs: 156-181, 1563-1581, or 3163-3185; a hinge domain; a transmembrane domain; and an intracellular signaling domain.

In several embodiments, the anti-BCMA binding moiety further comprises an additional VH comprising an additional HCDR1, HCDR2, and HCDR3, wherein the additional HCDR1 comprises a sequence having at least 85%, 90%, 91%, 92%, 93%, 94%, 95%, 96%, 97%, 98%, 99%, or 100% identity to any one of SEQ ID NOs: 3117-3139, the additional HCDR2 comprises a sequence having at least 85%, 90%, 91%, 92%, 93%, 94%, 95%, 96%, 97%, 98%, 99%, or 100% identity to any one of SEQ ID NOs: 3140-3162; and the additional HCDR3 comprises a sequence having at least 85%, 90%, 91%, 92%, 93%, 94%, 95%, 96%, 97%, 98%, 99%, or 100% identity to any one of SEQ ID NOs: 3163-3185. In several embodiments, other sequences are used for the additional HCDR1, HCDR2, and/or HCDR3.

In several embodiments, the intracellular signaling domain comprises comprising a co-stimulatory subdomain and a CD3ζ subdomain.

Also provided for herein is a population of engineered immune cells engineered to express a BCMA-directed chimeric antigen receptor (CAR), the CAR comprising an extracellular anti-BCMA binding moiety comprising a heavy chain variable region (VH) comprising a HCDR1, HCDR2, and HCDR3, wherein the HCDR1 comprises a sequence having at least 85%, 90%, 91%, 92%, 93%, 94%, 95%, 96%, 97%, 98%, 99%, or 100% identity to any one of SEQ ID NOs: 104-129, 1525-1543, or 3117-3139, the HCDR2 comprises a sequence having at least 85%, 90%, 91%, 92%, 93%, 94%, 95%, 96%, 97%, 98%, 99%, or 100% identity to any one of SEQ ID NOs: 130-155, 1544-1562, or 3140-3162; the HCDR3 comprises a sequence having at least 85%, 90%, 91%, 92%, 93%, 94%, 95%, 96%, 97%, 98%, 99%, or 100% identity to any one of SEQ ID NOs: 156-181, 1563-1581, or 3163-3185, and a hinge domain, a transmembrane domain; and an intracellular signaling domain. In several embodiments, the CAR expressed by the immune cells further comprises an additional VH comprising an additional HCDR1, HCDR2, and HCDR3, wherein the additional HCDR1 comprises a sequence having at least 85%, 90%, 91%, 92%, 93%, 94%, 95%, 96%, 97%, 98%, 99%, or 100% identity to any one of SEQ ID NOs: 3117-3139, the additional HCDR2 comprises a sequence having at least 85%, 90%, 91%, 92%, 93%, 94%, 95%, 96%, 97%, 98%, 99%, or 100% identity to any one of SEQ ID NOs: 3140-3162, and the additional HCDR3 comprises a sequence having at least 85%, 90%, 91%, 92%, 93%, 94%, 95%, 96%, 97%, 98%, 99%, or 100% identity to any one of SEQ ID NOs: 3163-3185. Other sequences may be used for the additional HCDR1, HCDR2, and/or HCDR3, depending on the embodiment. In several embodiments, the HCDR1 is encoded by a nucleic sequence having at least 85%, 90%, 91%, 92%, 93%, 94%, 95%, 96%, 97%, 98%, 99%, or 100% identity to any one of SEQ ID NOs: 182-207 or 3186-3208, the HCDR2 is encoded by a nucleic sequence having at least 85%, 90%, 91%, 92%, 93%, 94%, 95%, 96%, 97%, 98%, 99%, or 100% identity to any one of SEQ ID NOs: 208-233 or 3209-3231, and the HCDR3 is encoded by a nucleic sequence having at least 85%, 90%, 91%, 92%, 93%, 94%, 95%, 96%, 97%, 98%, 99%, or 100% identity to any one of SEQ ID NOs: 234-259 or 3232-3254.

In several embodiments, the CAR further comprises an a light chain variable region (VL) comprising an LCDR1, LCDR2, and LCDR3, wherein the LCDR1 comprises a sequence having at least 85%, 90%, 91%, 92%, 93%, 94%, 95%, 96%, 97%, 98%, 99%, or 100% identity to any one of SEQ ID NOs: 3117-3139, the LCDR2 comprises a sequence having at least 85%, 90%, 91%, 92%, 93%, 94%, 95%, 96%, 97%, 98%, 99%, or 100% identity to any one of SEQ ID NOs: 3140-3162, and the LCDR3 comprises a sequence having at least 85%, 90%, 91%, 92%, 93%, 94%, 95%, 96%, 97%, 98%, 99%, or 100% identity to any one of SEQ ID NOs: 3163-3185.

Also provided for herein is a population of immune cells engineered to express a BCMA-directed chimeric antigen receptor (CAR), the CAR comprising an anti-BCMA binding moiety comprising a heavy chain variable region (VH) comprising a HCDR1, HCDR2, and HCDR3, and a light chain variable region (VL) comprising an LCDR1, LCDR2, and LCDR3, wherein: the HCDR1 comprises a sequence having at least 85%, 90%, 91%, 92%, 93%, 94%, 95%, 96%, 97%, 98%, 99%, or 100% identity to any one of SEQ ID NOs: 114, 105, 107, 129, 104, 106, 108-113, or 115-128, the HCDR2 comprises a sequence having at least 85%, 90%, 91%, 92%, 93%, 94%, 95%, 96%, 97%, 98%, 99%, or 100% identity to any one of SEQ ID NOs: 140, 131, 133, 155, 130, 132, 134-139, or 141-154, the HCDR3 comprises a sequence having at least 85%, 90%, 91%, 92%, 93%, 94%, 95%, 96%, 97%, 98%, 99%, or 100% identity to any one of SEQ ID NOs: 166, 157, 159, 181, 156, 158, 160-165, or 167-180; the LCDR1 comprises a sequence having at least 85%, 90%, 91%, 92%, 93%, 94%, 95%, 96%, 97%, 98%, 99%, or 100% identity to any one of SEQ ID NOs: 395, 386, 388, 410, 385, 387, 389-392, or 396-409, the LCDR2 comprises a sequence having at least 85%, 90%, 91%, 92%, 93%, 94%, 95%, 96%, 97%, 98%, 99%, or 100% identity to any one of SEQ ID NOs: 421, 412 414, 436, 411, 413, 415-420, or 422-435, the LCDR3 comprises a sequence having at least 85%, 90%, 91%, 92%, 93%, 94%, 95%, 96%, 97%, 98%, 99%, or 100% identity to any one of SEQ ID NOs: 447, 438, 440, 462, 437, 439, 441-446, or 448-446.

In several embodiments, there is provided a method of treating a cancer, the method comprising administering to a subject in need thereof a population of immune cells comprising a BCMA-directed chimeric antigen receptor (CAR), the CAR comprising a heavy chain variable region (VH) comprising a HCDR1, HCDR2, and HCDR3, and a light chain variable region (VL) comprising an LCDR1, LCDR2, and LCDR3, wherein the HCDR1 comprises a sequence having at least 85%, 90%, 91%, 92%, 93%, 94%, 95%, 96%, 97%, 98%, 99%, or 100% identity to any one of SEQ ID NOs: 114, 105, 107, 129, 104, 106, 108-113, or 115-128, the HCDR2 comprises a sequence having at least 85%, 90%, 91%, 92%, 93%, 94%, 95%, 96%, 97%, 98%, 99%, or 100% identity to any one of SEQ ID NOs: 140, 131, 133, 155, 130, 132, 134-139, or 141-154, the HCDR3 comprises a sequence having at least 85%, 90%, 91%, 92%, 93%, 94%, 95%, 96%, 97%, 98%, 99%, or 100% identity to any one of SEQ ID NOs: 166, 157, 159, 181, 156, 158, 160-165, or 167-180, the LCDR1 comprises a sequence having at least 85%, 90%, 91%, 92%, 93%, 94%, 95%, 96%, 97%, 98%, 99%, or 100% identity to any one of SEQ ID NOs: 395, 386, 388, 410, 385, 387, 389-392, or 396-409, the LCDR2 comprises a sequence having at least 85%, 90%, 91%, 92%, 93%, 94%, 95%, 96%, 97%, 98%, 99%, or 100% identity to any one of SEQ ID NOs: 421, 412 414, 436, 411, 413, 415-420, or 422-435, the LCDR3 comprises a sequence having at least 85%, 90%, 91%, 92%, 93%, 94%, 95%, 96%, 97%, 98%, 99%, or 100% identity to any one of SEQ ID NOs: 447, 438, 440, 462, 437, 439, 441-446, or 448-446 and a hinge domain, a transmembrane domain, and an intracellular signaling domain comprising a CD3ζ subdomain. In several embodiments, the VH comprises a sequence having at least 85%, 90%, 91%, 92%, 93%, 94%, 95%, 96%, 97%, 98%, 99%, or 100% identity to any one of SEQ ID NOs: 270, 261, 263, 285, 260, 262, 263-269, or 271-284 and the VL comprises a sequence having at least 85%, 90%, 91%, 92%, 93%, 94%, 95%, 96%, 97%, 98%, 99%, or 100% identity to any one of SEQ ID NOs: 551, 542, 544, 566, 541, 543, 545-550, or 552-565. In several embodiments, the BCMA-directed CAR comprises a sequence having at least 85%, 90%, 91%, 92%, 93%, 94%, 95%, 96%, 97%, 98%, 99%, or 100% identity to any one of SEQ ID NOs: 3897, 3888, 3890, 3912, 3887, 3889, 3891-3896, or 3898-3911. In several embodiments, the BCMA-directed CAR comprises a sequence having at least 85%, 90%, 91%, 92%, 93%, 94%, 95%, 96%, 97%, 98%, 99%, or 100% identity to any one of SEQ ID NOs: 4053, 4044, 4046, 4068, 4043, 4045, 4047-4052, or 4054-4067. In several embodiments, the BCMA-directed CAR comprises a sequence having at least 85%, 90%, 91%, 92%, 93%, 94%, 95%, 96%, 97%, 98%, 99%, or 100% identity to any one of SEQ ID NOs: 3869, 3867, 3868, or 3870. In several embodiments, the methods (and uses of the population of immune cells is for the treatment of a cancer, such as multiple myeloma.

Disclosed herein are anti-BCMA binding moieties comprising a heavy chain variable region (VH) comprising a HCDR1, HCDR2, and HCDR3, and a light chain variable region (VL) comprising an LCDR1, LCDR2, and LCDR3. In some embodiments of the anti-BCMA binding moieties, the HCDR1 comprises a sequence having at least 85%, 90%, 91%, 92%, 93%, 94%, 95%, 96%, 97%, 98%, 99%, or 100% identity to any one of SEQ ID NOs: 104-129, the HCDR2 comprises a sequence having at least 85%, 90%, 91%, 92%, 93%, 94%, 95%, 96%, 97%, 98%, 99%, or 100% identity to any one of SEQ ID NOs: 130-155, and the HCDR3 comprises a sequence having at least 85%, 90%, 91%, 92%, 93%, 94%, 95%, 96%, 97%, 98%, 99%, or 100% identity to any one of SEQ ID NOs: 156-181. In some embodiments of the anti-BCMA binding moieties, the LCDR1 comprises a sequence having at least 85%, 90%, 91%, 92%, 93%, 94%, 95%, 96%, 97%, 98%, 99%, or 100% identity to any one of SEQ ID NOs: 385-410, the LCDR2 comprises a sequence having at least 85%, 90%, 91%, 92%, 93%, 94%, 95%, 96%, 97%, 98%, 99%, or 100% identity to any one of SEQ ID NOs: 411-436, and the LCDR3 comprises a sequence having at least 85%, 90%, 91%, 92%, 93%, 94%, 95%, 96%, 97%, 98%, 99%, or 100% identity to any one of SEQ ID NOs: 437-462. In some embodiments, the VH comprises a sequence having at least 85%, 90%, 91%, 92%, 93%, 94%, 95%, 96%, 97%, 98%, 99%, or 100% identity to any one of SEQ ID NOs: 260-285. In some embodiments, the VL comprises a sequence having at least 85%, 90%, 91%, 92%, 93%, 94%, 95%, 96%, 97%, 98%, 99%, or 100% identity to any one of SEQ ID NOs: 541-566.

Also disclosed herein are BCMA-directed chimeric antigen receptors comprising an extracellular anti-BCMA binding moiety, a hinge domain, a transmembrane domain, and an intracellular signaling domain comprising an OX40 subdomain and a CD3ζ subdomain. In some embodiments, the anti-BCMA binding moiety is any one of the anti-BCMA binding moieties disclosed herein. In some embodiments, the OX40 subdomain comprises a sequence having at least 85%, 90%, 91%, 92%, 93%, 94%, 95%, 96%, 97%, 98%, 99%, or 100% identity to SEQ ID NO: 1394. In some embodiments, the CD3ζ subdomain comprises a sequence having at least 85%, 90%, 91%, 92%, 93%, 94%, 95%, 96%, 97%, 98%, 99%, or 100% identity to SEQ ID NO: 1395. In some embodiments, the transmembrane domain is a CD8 transmembrane domain. In some embodiments, the hinge domain is a CD8 hinge domain, an IgG4 hinge domain, or an RQRCD8 hinge domain.

Also disclosed herein are BCMA-directed CAR constructs comprising a BCMA-directed CAR and a membrane-bound IL15 (mbIL15). In some embodiments, the BCMA-directed CAR is any one of the BCMA-directed CARs disclosed herein. In some embodiments, the mbIL15 comprises a sequence having at least 85%, 90%, 91%, 92%, 93%, 94%, 95%, 96%, 97%, 98%, 99%, or 100% identity to SEQ ID NO: 1398.

Also disclosed herein are immune cells or populations of immune cells comprising any one of the anti-BCMA binding moieties, BCMA-directed CARs, or BCMA-directed CAR constructs disclosed herein. In some embodiments, the immune cells are NK cells and/or T cells.

Also disclosed herein are methods of treating cancer in a subject in need thereof. In some embodiments, the methods comprise administering to the subject any one of the anti-BCMA binding moieties, BCMA-directed CARs, BCMA-directed CAR constructs, or engineered immune cells disclosed herein. Also disclosed herein are any one of the anti-BCMA binding moieties, BCMA-directed CARs, BCMA-directed CAR constructs, or engineered immune cells disclosed herein for use in treating cancer. Also disclosed herein are any one of the anti-BCMA binding moieties, BCMA-directed CARs, BCMA-directed CAR constructs, or engineered immune cells disclosed herein for use in the manufacture of a medicament.

### BRIEF DESCRIPTION OF THE DRAWINGS

FIGS. 1A-1D depict a collection of non-limiting embodiments of CAR constructs used, for example, to target BCMA. The constructs depicted include the membrane-bound IL15 construct separated from the CAR per se by a self-cleaving peptide (e.g. T2A). However, CARs excluding the IL15 construct (and separating self-cleaving peptide) are also provided for herein. “CD8 SP”: CD8 signal peptide; “GS linker”: linker comprising glycine and serine; “VH”: heavy chain variable region; “VL”: light chain variable region”; “CD8 TM”: CD8 transmembrane domain; “CD8 IC”: CD8 intracellular domain; “OX40”: OX40 intracellular domain; “CD3ζ”: CD3ζ domain. FIG. 1A shows VH-VL format. FIG. 1B shows a VL-VH format. FIG. 1C shows a VH-VL format with a shorter hinge (IgG4). FIG. 1D shows a VH-VL format with a longer hinge (RQRCD8). It shall be appreciated that these represent the nucleotide sequence and the encoded CAR construct would not include the T2A and the mbIL15, if encoded, would be expressed separately on the cell.

FIG. 2 depicts non-limiting combinations of heavy chain variable region (VH) complementarity-determining regions (CDRs) 1, 2 and 3 (HCDR1, HCDR2, and HCDR3) sequences. Embodiments of the anti-BCMA binding moieties, BCMA-directed CARs, and cells expressing thereof may use any of the non-limiting combinations depicted herein.

FIG. 3 depicts non-limiting combinations of light chain variable region (VL) CDRs 1, 2, and 3 (LCDR1, LCDR2, and LCDR3) sequences. Embodiments of the anti-BCMA binding moieties, BCMA-directed CARs, and cells expressing thereof may use any of the non-limiting combinations depicted herein.

FIG. 4 depicts non-limiting combinations of VH and VL sequences. Embodiments of the anti-BCMA binding moieties, BCMA-directed CARs, and cells expressing thereof may use any of the non-limiting combinations depicted herein.

FIGS. 5A-5D show schematics of various non-limiting embodiments of CAR constructs provided for herein, for example, to target BCMA. FIG. 5A shows a non-limiting schematic of a VH-GS linker-VL scFv, with CD8a hinge, CD8α transmembrane domain, CD8α intracellular domain, an OX-40 co-stimulatory domain, and a CD3ζ signaling domain. Also included in the schematic, though not used in all embodiments provided for herein, is a T2A self-cleaving peptide followed by additional nucleotides encoding mbIL15. FIG. 5B shows the encoded amino acid. FIGS. 5C and 5D show schematics of corresponding constructs, without a FLAG tag-linker complex.

FIGS. 6A-6D show schematics of various non-limiting embodiments of CAR constructs provided for herein, for example, to target BCMA. FIG. 6A shows a non-limiting schematic of a VH-Whitlow linker-VL scFv, AAA spacer, with a CD8α hinge, CD8α transmembrane domain, CD8α intracellular domain, an OX-40 co-stimulatory domain, and a CD3ζ signaling domain. Also included in the schematic, though not used in all embodiments provided for herein, is a T2A self-cleaving peptide followed by additional nucleotides encoding mbIL15. FIG. 6B shows the encoded amino acid. FIGS. 6C and 6D show schematics of corresponding constructs, without a FLAG tag-linker complex.

FIGS. 7A-7D show schematics of various non-limiting embodiments of CAR constructs provided for herein, for example, to target BCMA. FIG. 7A shows a non-limiting schematic of a VL-Whitlow linker-VH scFv, AAA spacer, with a CD8α hinge, CD8α transmembrane domain, CD8α intracellular domain, an OX-40 co-stimulatory domain, and a CD3ζ signaling domain. Also included in the schematic, though not used in all embodiments provided for herein, is a T2A self-cleaving peptide followed by additional nucleotides encoding mbIL15. FIG. 7B shows the encoded amino acid. FIGS. 7C and 7D show schematics of corresponding constructs, without a FLAG tag-linker complex.

FIGS. 8A-8D show schematics of various non-limiting embodiments of CAR constructs provided for herein, for example, to target BCMA. FIG. 8A shows a non-limiting schematic of a camelid VHH domain, with a CD8α hinge, CD8α transmembrane domain, CD8α intracellular domain, an OX-40 co-stimulatory domain, and a CD3ζ signaling domain. Also included in the schematic, though not used in all embodiments provided for herein, is a T2A self-cleaving peptide followed by additional nucleotides encoding mbIL15. FIG. 8B shows the encoded amino acid. FIGS. 8C and 8D show schematics of corresponding constructs, without a FLAG tag-linker complex.

FIGS. 9A-9D show schematics of various non-limiting embodiments of CAR constructs provided for herein, for example, to target BCMA. FIG. 9A shows a non-limiting schematic of a camelid VHH domain-GS linker-VHH domain, with a CD8α hinge, CD8α transmembrane domain, CD8α intracellular domain, an OX-40 co-stimulatory domain, and a CD3ζ signaling domain. The VHH domains may be the same, or different domains. Also included in the schematic, though not used in all embodiments provided for herein, is a T2A self-cleaving peptide followed by additional nucleotides encoding mbIL15. FIG. 9B shows the encoded amino acid. FIGS. 9C and 9D show schematics of corresponding constructs, without a FLAG tag-linker complex.

FIGS. 10A-10D show schematics of various non-limiting embodiments of CAR constructs provided for herein, for example, to target BCMA. FIG. 10A shows a non-limiting schematic of a VL-Whitlow linker-VH scFv, AAA spacer, with a CD8α hinge, CD8α transmembrane domain, CD8α intracellular domain, an OX-40 co-stimulatory domain, and a CD3ζ signaling domain. Also included in the schematic, though not used in all embodiments provided for herein, is a T2A self-cleaving peptide followed by additional nucleotides encoding mbIL15. FIG. 10B shows the encoded amino acid. FIGS. 10C and 10D show schematics of corresponding constructs, without a FLAG tag-linker complex.

FIGS. 11A-11D show schematics of various non-limiting embodiments of CAR constructs provided for herein, for example, to target BCMA. FIG. 11A shows a non-limiting schematic of a VL-Whitlow-VH, AAA spacer, with a CD28 hinge, CD28 transmembrane domain, CD28 intracellular domain, and a CD3ζ signaling domain. Also included in the schematic, though not used in all embodiments provided for herein, is a T2A self-cleaving peptide followed by additional nucleotides encoding mbIL15. FIG. 11B shows the encoded amino acid. FIGS. 11C and 11D show schematics of corresponding constructs, without a FLAG tag-linker complex.

FIGS. 12A-12D show schematics of various non-limiting embodiments of CAR constructs provided for herein, for example, to target BCMA. FIG. 12A shows a non-limiting schematic of a VH-GS linker-VL scFv, AAA spacer, with a CD28 hinge, CD28 transmembrane domain, CD28 intracellular domain, and a CD3ζ signaling domain. Also included in the schematic, though not used in all embodiments provided for herein, is a T2A self-cleaving peptide followed by additional nucleotides encoding mbIL15. FIG. 12B shows the encoded amino acid. FIGS. 12C and 12D show schematics of corresponding constructs, without a FLAG tag-linker complex.

FIGS. 13A-13D show schematics of various non-limiting embodiments of CAR constructs provided for herein, for example, to target BCMA. FIG. 13A shows a non-limiting schematic of a VH-GS linker-VL scFv, AAA spacer, with a CD8α hinge, CD8α transmembrane domain, CD8α intracellular domain, a 4-1BB co-stimulatory domain, and a CD3ζ signaling domain. Also included in the schematic, though not used in all embodiments provided for herein, is a T2A self-cleaving peptide followed by additional nucleotides encoding mbIL15. FIG. 13B shows the encoded amino acid. FIGS. 13C and 13D show schematics of corresponding constructs, without a FLAG tag-linker complex.

FIGS. 14A-14D show schematics of various non-limiting embodiments of CAR constructs provided for herein, for example, to target BCMA. FIG. 14A shows a non-limiting schematic of a VL-Whitlow-VH scFv, AAA spacer, with a CD28 hinge, CD28 transmembrane domain, CD28 intracellular domain, and a CD3ζ signaling domain. Also included in the schematic, though not used in all embodiments provided for herein, is a T2A self-cleaving peptide followed by additional nucleotides encoding mbIL15. FIG. 14B shows the encoded amino acid. FIGS. 14C and 14D show schematics of corresponding constructs, without a FLAG tag-linker complex.

FIGS. 15A-15D show schematics of various non-limiting embodiments of CAR constructs provided for herein, for example, to target BCMA. FIG. 15A shows a non-limiting schematic of a VL-Whitlow-VH scFv, AAA spacer, with a CD8α hinge, CD8α transmembrane domain, CD8α intracellular domain, a 4-1BB co-stimulatory domain, and a CD3ζ signaling domain. Also included in the schematic, though not used in all embodiments provided for herein, is a T2A self-cleaving peptide followed by additional nucleotides encoding mbIL15. FIG. 15B shows the encoded amino acid. FIGS. 15C and 15D show schematics of corresponding constructs, without a FLAG tag-linker complex.

FIGS. 16A-16D show schematics of various non-limiting embodiments of CAR constructs provided for herein, for example, to target BCMA. FIG. 16A shows a non-limiting schematic of a VH-GS linker-VL scFv, AAA spacer, with a CD8α hinge, CD8α transmembrane domain, CD8α intracellular domain, a 4-1BB co-stimulatory domain, and a CD3ζ signaling domain. Also included in the schematic, though not used in all embodiments provided for herein, is a T2A self-cleaving peptide followed by additional nucleotides encoding mbIL15. FIG. 16B shows the encoded amino acid. FIGS. 16C and 16D show schematics of corresponding constructs, without a FLAG tag-linker complex.

FIGS. 17A-17D show schematics of various non-limiting embodiments of CAR constructs provided for herein, for example, to target BCMA. FIG. 17A shows a non-limiting schematic of a VL-Whitlow linker-VH scFv, (AAA)₂ spacer, with a CD8α hinge, CD8α transmembrane domain, CD8α intracellular domain, a 4-1BB co-stimulatory domain, and a CD3ζ signaling domain. Also included in the schematic, though not used in all embodiments provided for herein, is a T2A self-cleaving peptide followed by additional nucleotides encoding mbIL15. FIG. 17B shows the encoded amino acid. FIGS. 17C and 17D show schematics of corresponding constructs, without a FLAG tag-linker complex.

FIGS. 18A-18D show schematics of various non-limiting embodiments of CAR constructs provided for herein, for example, to target BCMA. FIG. 18A shows a non-limiting schematic of a multi-domain camelid BCMA binder with a VHH-GS linker-VHH structure, AAA spacer, with a CD8α hinge, CD8α transmembrane domain, CD8α intracellular domain, a 4-1BB co-stimulatory domain, and a CD3ζ signaling domain. Also included in the schematic, though not used in all embodiments provided for herein, is a T2A self-cleaving peptide followed by additional nucleotides encoding mbIL15. FIG. 18B shows the encoded amino acid. FIGS. 18C and 18D show schematics of corresponding constructs, without a FLAG tag-linker complex.

FIG. 19 shows a dot plot depicting expression of various non-limiting examples of CAR formats in Jurkat cells.

FIG. 20 depicts data related to the function of a non-limiting embodiment of a BMCA-directed CAR. This figure shows data related to the assessment of the tonic activation (Y axis) versus BCMA-binding induced activation (X-axis) when target MM.1S cells were co-cultured with Jurkat cells engineered to express a BCMA-directed CAR as provided for herein. The effector to target (E:T) cell ratio used was 1:1 (Jurkat:MM.1S).

FIG. 21 shows data related to the ratio of BCMA-induced activation to tonic activation of various non-limiting embodiments of CARs as provided for herein.

FIG. 22 shows additional data related to the expression (left panel) of various clones of a CAR, each individual clone varying in sequence, but sharing a common CAR structure. The right panel of FIG. 22 shows BCMA binding for each clone.

FIGS. 23A-23B show scatterplots of activity versus CAR expression. FIG. 23A shows a scatterplot of the BCMA-binding induced CAR activation versus CAR expression. FIG. 23B shows a scatterplot of tonic signaling versus CAR expression.

FIG. 24 shows a scatterplot of BCMA binding versus CAR expression.

FIG. 25 shows a scatterplot of the activation/tonic signaling ratio of various CAR constructs provided for herein versus

FIGS. 26A-26F show data related to the function of CARs provided for herein based on one aspect of their structure, the linker. FIG. 26A shows data related to the activation/tonic signaling ratio for CARs using a Whitlow linker versus a GS linker. FIG. 26B shows expression data for CARs using a Whitlow linker versus a GS linker. FIG. 26C shows data related to the activation/tonic signaling ratio for CARs provided for herein using scFvs employing a VH-VL format versus a VL-VH format. FIG. 26D shows data related to the expression of CARs provided for herein using scFvs employing a VH-VL format versus a VL-VH format. FIG. 26E shows data related to the activation/tonic signaling ratio for CARs provided for herein using CD28 domains (hinge, transmembrane, and intracellular) versus CARs using CD8 domains with an OX-40 co-stimulatory domain.

FIG. 27 shows summary data related to the expression and BCMA binding function of monovalent or bivalent CARs using one or more camelid VHH domains.

FIGS. 28A-28B show data for expression and BCMA-binding function of monovalent and bivalent CARs using one or more camelid VHH domains. FIG. 28A shows expression data for bivalent (Y axis) versus monovalent CARs using one or more camelid VHH domains. FIG. 28B shows BCMA binding data for bivalent (Y axis) versus monovalent VHH-CARs.

FIGS. 29A-29D show assessment of various characteristics of cells expressing CARs employing one or more VHH domains. FIG. 29A shows a scatterplot of tonic signaling activity for bivalent (Y axis) versus monovalent (X axis) CARs comprising one or more VHH domains. FIG. 29B shows a scatterplot of the activation/tonic signaling ratio for bivalent (Y axis) versus monovalent (X axis) VHH-CARs. FIG. 29C is a histogram showing the activation/tonic signaling ratio for selected bivalent (closed bars) and monovalent (open bars) VHH-CARs. FIG. 29D shows data related to the tonic signaling detected from selected bivalent (closed bars) and monovalent (open bars) VHH-CARs.

FIGS. 30A-30B show data comparing different CAR constructs. FIG. 30A shows a scatterplot of tonic signaling versus expression for CARs using scFv (circle) versus VHH-CARs (triangle) and corresponding controls (scFv is square, VHH is an X).

### DETAILED DESCRIPTION

Some embodiments of the methods and compositions provided herein relate to anti-BCMA binding moieties. Also disclosed herein are BCMA-directed chimeric antigen receptors (CARs) comprising any of the anti-BCMA binding moieties disclosed herein. In some embodiments, the CARs are expressed on a cell as described herein. Some embodiments include methods of use of the compositions or cells in immunotherapy. Some embodiments relate to use of anti-BCMA CARs expressed on Natural Killer (NK) cells.

The term “anticancer effect” refers to a biological effect which can be manifested by various means, including but not limited to, a decrease in tumor volume, a decrease in the number of cancer cells, a decrease in the number of metastases, an increase in life expectancy, decrease in cancer cell proliferation, decrease in cancer cell survival, or amelioration of various physiological symptoms associated with the cancerous condition. An “anticancer effect” can also be manifested by the ability of the CARs in prevention of the occurrence of cancer in the first place.

### Cell Types

Some embodiments of the methods and compositions provided herein relate to a cell such as an immune cell. For example, an immune cell may be engineered to include a chimeric antigen receptor such as a BCMA-directed CAR, or engineered to include a nucleic acid encoding said CAR as described herein.

Traditional anti-cancer therapies relied on a surgical approach, radiation therapy, chemotherapy, or combinations of these methods. As research led to a greater understanding of some of the mechanisms of certain cancers, this knowledge was leveraged to develop targeted cancer therapies. Targeted therapy is a cancer treatment that employs certain drugs that target specific genes or proteins found in cancer cells or cells supporting cancer growth, (like blood vessel cells) to reduce or arrest cancer cell growth. More recently, genetic engineering has enabled approaches to be developed that harness certain aspects of the immune system to fight cancers. In some cases, a patient's own immune cells are modified to specifically eradicate that patient's type of cancer. Various types of immune cells can be used, such as T cells and/or Natural Killer (NK cells), as described in more detail herein.

To facilitate cancer immunotherapies, there are provided for herein polynucleotides, polypeptides, and vectors that encode chimeric antigen receptors (CAR) that comprise a target binding moiety (e.g., an extracellular binder of a ligand expressed by a cancer cell, such as a BCMA-directed chimeric antigen receptor) and a cytotoxic signaling complex. Some embodiments include a polynucleotide, polypeptide, or vector that encodes a BCMA-directed chimeric antigen receptor to facilitate targeting of an immune cell to a cancer and exerting cytotoxic effects on the cancer cell. Also provided are engineered immune cells (e.g., T cells and/or NK cells) expressing such CARs. There are also provided herein, in several embodiments, polynucleotides, polypeptides, and vectors that encode a construct comprising an extracellular domain comprising two or more subdomains and a cytotoxic signaling complex. Also provided are engineered immune cells (e.g., T cells and/or NK cells) expressing such bi-specific constructs. Methods of treating cancer and other uses of such cells for cancer immunotherapy are also provided for herein.

### Engineered Cells for Immunotherapy

In several embodiments, cells of the immune system are engineered to have enhanced cytotoxic effects against target cells, such as tumor cells. For example, a cell of the immune system may be engineered to include a BCMA-directed chimeric antigen receptor as described herein. In several embodiments, white blood cells or leukocytes, are used, since their native function is to defend the body against growth of abnormal cells and infectious disease. There are a variety of types of white bloods cells that serve specific roles in the human immune system, and are therefore a preferred starting point for the engineering of cells disclosed herein. White blood cells include granulocytes and agranulocytes (presence or absence of granules in the cytoplasm, respectively). Granulocytes include basophils, eosinophils, neutrophils, and mast cells. Agranulocytes include lymphocytes and monocytes. Cells such as those that follow or are otherwise described herein may be engineered to include a chimeric antigen receptor such as a BCMA-directed chimeric antigen receptor, or a nucleic acid encoding such chimeric antigen receptor and/or engineered to co-express a membrane-bound interleukin 15 (mbIL15) co-stimulatory domain.

### Monocytes for Immunotherapy

Monocytes are a subtype of leukocyte. Monocytes can differentiate into macrophages and myeloid lineage dendritic cells. Monocytes are associated with the adaptive immune system and serve the main functions of phagocytosis, antigen presentation, and cytokine production. Phagocytosis is the process of uptake cellular material, or entire cells, followed by digestion and destruction of the engulfed cellular material. In several embodiments, monocytes are used in connection with one or more additional engineered cells as disclosed herein. Some embodiments of the methods and compositions described herein relate to a monocyte that includes a BCMA-directed chimeric antigen receptor, or a nucleic acid encoding the BCMA-directed chimeric antigen receptor. Several embodiments of the methods and compositions disclosed herein relate to monocytes engineered to express a BCMA-directed chimeric antigen receptor and a membrane-bound interleukin 15 (mbIL15) co-stimulatory domain.

### Lymphocytes for Immunotherapy

Lymphocytes, the other primary sub-type of leukocyte include T cells (cell-mediated, cytotoxic adaptive immunity), natural killer cells (cell-mediated, cytotoxic innate immunity), and B cells (humoral, antibody-driven adaptive immunity). While B cells are engineered according to several embodiments, disclosed herein, several embodiments also relate to engineered T cells or engineered NK cells (mixtures of T cells and NK cells are used in some embodiments). Some embodiments of the methods and compositions described herein relate to a lymphocyte that includes a BCMA-directed chimeric antigen receptor, or a nucleic acid encoding the BCMA-directed chimeric antigen receptor. Several embodiments of the methods and compositions disclosed herein relate to lymphocytes engineered to express a BCMA-directed chimeric antigen receptor and a membrane-bound interleukin 15 (mbIL15) co-stimulatory domain.

### T Cells for Immunotherapy

T cells are distinguishable from other lymphocytes sub-types (e.g., B cells or NK cells) based on the presence of a T cell receptor on the cell surface. T cells can be divided into various different subtypes, including effector T cells, helper T cells, cytotoxic T cells, memory T cells, regulatory T cells, natural killer T cell, mucosal associated invariant T cells and gamma delta T cells. In some embodiments, a specific subtype of T cell is engineered. In some embodiments, a mixed pool of T cell subtypes is engineered. In some embodiments, there is no specific selection of a type of T cells to be engineered to express the cytotoxic receptor complexes disclosed herein. In several embodiments, specific techniques, such as use of cytokine stimulation are used to enhance expansion/collection of T cells with a specific marker profile. For example, in several embodiments, activation of certain human T cells, e.g. CD4+ T cells, CD8+ T cells is achieved through use of CD3 and/or CD28 as stimulatory molecules. In several embodiments, there is provided a method of treating or preventing cancer or an infectious disease, comprising administering a therapeutically effective amount of T cells expressing the cytotoxic receptor complex and/or a homing moiety as described herein. In several embodiments, the engineered T cells are autologous cells, while in some embodiments, the T cells are allogeneic cells. Some embodiments of the methods and compositions described herein relate to a T cell that includes a BCMA-directed chimeric antigen receptor, or a nucleic acid encoding the BCMA-directed chimeric antigen receptor. Several embodiments of the methods and compositions disclosed herein relate to T cells engineered to express a BCMA-directed chimeric antigen receptor and a membrane-bound interleukin 15 (mbIL15) co-stimulatory domain.

### NK Cells for Immunotherapy

In several embodiments, there is provided a method of treating or preventing cancer or an infectious disease, comprising administering a therapeutically effective amount of natural killer (NK) cells expressing the cytotoxic receptor complex and/or a homing moiety as described herein. In several embodiments, the engineered NK cells are autologous cells, while in some embodiments, the NK cells are allogeneic cells. In several embodiments, NK cells are preferred because the natural cytotoxic potential of NK cells is relatively high. In several embodiments, it is unexpectedly beneficial that the engineered cells disclosed herein can further upregulate the cytotoxic activity of NK cells, leading to an even more effective activity against target cells (e.g., tumor or other diseased cells). In several embodiments, the high degree of acute cytotoxicity of NK cells (which is further enhanced by the engineering methods disclosed herein) is leveraged to provide particularly efficacious cellular therapy compositions. Some embodiments of the methods and compositions described herein relate to an NK that includes a BCMA-directed chimeric antigen receptor, or a nucleic acid encoding the BCMA-directed chimeric antigen receptor. Several embodiments of the methods and compositions disclosed herein relate to NK cells engineered to express a BCMA-directed chimeric antigen receptor and a membrane-bound interleukin 15 (mbIL15) co-stimulatory domain. In some embodiments, the NK cells are derived from cell line NK-92. NK-92 cells are derived from NK cells, but lack major inhibitory receptors displayed by normal NK cells, while retaining the majority of activating receptors. Some embodiments of NK-92 cells described herein related to NK-92 cell engineered to silence certain additional inhibitory receptors, for example, SMAD3, allowing for upregulation of interferon-γ (IFNγ), granzyme B, and/or perforin production. Additional information relating to the NK-92 cell line is disclosed in WO 1998/49268 and U.S. Patent Application Publication No. 2002-0068044 and incorporated in their entireties herein by reference. NK-92 cells are used, in several embodiments, in combination with one or more of the other cell types disclosed herein. For example, in one embodiment, NK-92 cells are used in combination with NK cells as disclosed herein. In an additional embodiment, NK-92 cells are used in combination with T cells as disclosed herein.

### Hematopoietic Stem Cells for Cancer Immunotherapy

In some embodiments, hematopoietic stem cells (HSCs) are used in the methods of immunotherapy disclosed herein. In several embodiments, the cells are engineered to express a homing moiety and/or a cytotoxic receptor complex. HSCs are used, in several embodiments, to leverage their ability to engraft for long-term blood cell production, which could result in a sustained source of targeted anti-cancer effector cells, for example to combat cancer remissions. In several embodiments, this ongoing production helps to offset anergy or exhaustion of other cell types, for example due to the tumor microenvironment. In several embodiments, allogeneic HSCs are used, while in some embodiments, autologous HSCs are used. In several embodiments, HSCs are used in combination with one or more additional engineered cell type disclosed herein. Some embodiments of the methods and compositions described herein relate to a stem cell, such as a hematopoietic stem cell, that includes a BCMA-directed chimeric antigen receptor, or a nucleic acid encoding the BCMA-directed chimeric antigen receptor. Several embodiments of the methods and compositions disclosed herein relate to stem cells, such as hematopoietic stem cells that are engineered to express a BCMA-directed chimeric antigen receptor and a membrane-bound interleukin 15 (mbIL15) co-stimulatory domain.

### Induced Pluripotent Stem Cells for Cancer Immunotherapy

In some embodiments, induced pluripotent stem cells (iPSCs) are used in the method of immunotherapy disclosed herein. iPSCs are used, in several embodiments, to leverage their ability to differentiate and derive into non-pluripotent cells, including, but not limited to, CD34 cells, hemogenic endothelium cells, HSCs (hematopoietic stem and progenitor cells), hematopoietic multipotent progenitor cells, T cell progenitors, NK cell progenitors, T cells, NKT cells, NK cells, and B cells comprising one or several genetic modifications at selected sites through differentiating iPSCs or less differentiated cells comprising the same genetic modifications at the same selected sites. In several embodiments, the iPSCs are used to generate iPSC-derived NK or T cells. In several embodiments, the cells are engineered to express a homing moiety and/or a cytotoxic receptor complex. In several embodiments, iPSCs are used in combination with one or more additional engineered cell type disclosed herein. Some embodiments of the methods and compositions described herein relate to a stem cell, such as a induced pluripotent stem cell engineered to express a CAR that targets a tumor marker, for example, CD19, CD123, CD70, Her2, mesothelin, Claudin 6, BCMA, EGFR, among any of the others disclosed herein, and optionally a membrane-bound interleukin 15 (mbIL15) co-stimulatory domain. Several embodiments of the methods and compositions disclosed herein relate to induced pluripotent stem cells engineered to express an activating chimeric receptor that targets a ligand on a tumor cell, for example, MICA, MICB, ULBP1, ULBP2, ULBP3, ULBP4, ULBP5, and ULBP6 (among others) and optionally a membrane-bound interleukin 15 (mbIL15) co-stimulatory domain.

### Genetic Engineering of Immune Cells

As discussed above, a variety of cell types can be utilized in cellular immunotherapy. Further, as elaborated on in more detail below, and shown in the Examples, genetic modifications can be made to these cells in order to enhance one or more aspects of their efficacy (e.g., cytotoxicity) and/or persistence (e.g., active life span). As discussed herein, in several embodiments NK cells are used for immunotherapy. In several embodiments provided for herein, gene editing of the NK cell can advantageously impart to the edited NK cell the ability to resist and/or overcome various inhibitory signals that are generated in the tumor microenvironment. It is known that tumors generate a variety of signaling molecules that are intended to reduce the anti-tumor effects of immune cells. As discussed in more detail below, in several embodiments, gene editing of the NK cell limits this tumor microenvironment suppressive effect on the NK cells, T cells, combinations of NK and T cells, or any edited/engineered immune cell provided for herein. As discussed below, in several embodiments, gene editing is employed to reduce or knockout expression of target proteins, for example by disrupting the underlying gene encoding the protein. In several embodiments, gene editing can reduce expression of a target protein by about 30%, about 40%, about 50%, about 60%, about 70%, about 75%, about 80%, about 85%, about 90%, about 95%, about 97%, about 98%, about 99%, or more (including any amount between those listed). In several embodiments, the gene is completely knocked out, such that expression of the target protein is undetectable. In several embodiments, gene editing is used to “knock in” or otherwise enhance expression of a target protein. In several embodiments, expression of a target protein can be enhanced by about 30%, about 40%, about 50%, about 60%, about 70%, about 75%, about 80%, about 85%, about 90%, about 95%, about 97%, about 98%, about 99%, or more (including any amount between those listed).

By way of non-limiting example, TGF-beta is one such cytokine released by tumor cells that results in immune suppression within the tumor microenvironment. That immune suppression reduces the ability of immune cells, even engineered CAR-immune cells is some cases, to destroy the tumor cells, thus allowing for tumor progression. In several embodiments, as discussed in detail below, immune checkpoint inhibitors are disrupted through gene editing. In several embodiments, blockers of immune suppressing cytokines in the tumor microenvironment are used, including blockers of their release or competitive inhibitors that reduce the ability of the signaling molecule to bind and inhibit an immune cell. Such signaling molecules include, but are not limited to TGF-beta, IL10, arginase, inducible NOS, reactive-NOS, Arg1, Indoleamine 2,3-dioxygenase (IDO), and PGE2. However, in additional embodiments, there are provided immune cells, such as NK cells, wherein the ability of the NK cell (or other cell) to respond to a given immunosuppressive signaling molecule is disrupted and/or eliminated. For example, in several embodiments, in several embodiments, NK cells or T cells are genetically edited to become have reduced sensitivity to TGF-beta. TGF-beta is an inhibitor of NK cell function on at least the levels of proliferation and cytotoxicity. Thus, according to some embodiments, the expression of the TGF-beta receptor is knocked down or knocked out through gene editing, such that the edited NK is resistant to the immunosuppressive effects of TGF-beta in the tumor microenvironment. In several embodiments, the TGFB2 receptor is knocked down or knocked out through gene editing, for example, by use of CRISPR-Cas editing. Small interfering RNA, antisense RNA, TALENs or zinc fingers are used in other embodiments. Other isoforms of the TGF-beta receptor (e.g., TGF-beta 1 and/or TGF-beta 3) are edited in some embodiments. In some embodiments TGF-beta receptors in T cells are knocked down through gene editing.

In accordance with additional embodiments, other modulators of one or more aspects of NK cell (or T cell) function are modulated through gene editing. A variety of cytokines impart either negative (as with TGF-beta above) or positive signals to immune cells. By way of non-limiting example, IL15 is a positive regulator of NK cells, which as disclosed herein, can enhance one or more of NK cell homing, NK cell migration, NK cell expansion/proliferation, NK cell cytotoxicity, and/or NK cell persistence. To keep NK cells in check under normal physiological circumstances, a cytokine-inducible SH2-containing protein (CIS, encoded by the CISH gene) acts as a critical negative regulator of IL-15 signaling in NK cells. As discussed herein, because IL15 biology impacts multiple aspects of NK cell functionality, including, but not limited to, proliferation/expansion, activation, cytotoxicity, persistence, homing, migration, among others. Thus, according to several embodiments, editing CISH enhances the functionality of NK cells across multiple functionalities, leading to a more effective and long-lasting NK cell therapeutic. In several embodiments, inhibitors of CIS are used in conjunction with engineered NK cell administration. In several embodiments, the CIS expression is knocked down or knocked out through gene editing of the CISH gene, for example, by use of CRISPR-Cas editing. Small interfering RNA, antisense RNA, TALENs or zinc fingers are used in other embodiments. In some embodiments CIS expression in T cells is knocked down through gene editing.

In several embodiments, CISH gene editing endows an NK cell with enhanced ability to home to a target site. In several embodiments, CISH gene editing endows an NK cell with enhanced ability to migrate, e.g., within a tissue in response to, for example chemoattractants or away from repellants. In several embodiments, CISH gene editing endows an NK cell with enhanced ability to be activated, and thus exert, for example, anti-tumor effects. In several embodiments, CISH gene editing endows an NK cell with enhanced proliferative ability, which in several embodiments, allows for generation of robust NK cell numbers from a donor blood sample. In addition, in such embodiments, NK cells edited for CISH and engineered to express a CAR are more readily, robustly, and consistently expanded in culture. In several embodiments, CISH gene editing endows an NK cell with enhanced cytotoxicity. In several embodiments, the editing of CISH synergistically enhances the cytotoxic effects of engineered NK cells and/or engineered T cells that express a CAR.

In several embodiments, CISH gene editing activates or inhibits a wide variety of pathways. The CIS protein is a negative regulator of IL15 signaling by way of, for example, inhibiting JAK-STAT signaling pathways. These pathways would typically lead to transcription of IL15-responsive genes (including CISH). In several embodiments, knockdown of CISH disinhibits JAK-STAT (e.g., JAK1-STAT5) signaling and there is enhanced transcription of IL15-responsive genes. In several embodiments, knockout of CISH yields enhanced signaling through mammalian target of rapamycin (mTOR), with corresponding increases in expression of genes related to cell metabolism and respiration. In several embodiments, knockout of CISH yields IL15 induced increased expression of IL-2Rα (CD25), but not IL-15Rα or IL-2/15Rβ, enhanced NK cell membrane binding of IL15 and/or IL2, increased phosphorylation of STAT-3 and/or STAT-5, and elevated expression of the antiapoptotic proteins, such as Bcl-2. In several embodiments, CISH knockout results in IL15-induced upregulation of selected genes related to mitochondrial functions (e.g., electron transport chain and cellular respiration) and cell cycle. Thus, in several embodiments, knockout of CISH by gene editing enhances the NK cell cytotoxicity and/or persistence, at least in part via metabolic reprogramming. In several embodiments, negative regulators of cellular metabolism, such as TXNIP, are downregulated in response to CISH knockout. In several embodiments, promotors for cell survival and proliferation including BIRC5 (Survivin), TOP2A, CKS2, and RACGAP1 are upregulated after CISH knockout, whereas antiproliferative or proapoptotic proteins such as TGFB1, ATM, and PTCH1 are downregulated. In several embodiments, CISH knockout alters the state (e.g., activates or inactivates) signaling via or through one or more of CXCL-10, IL2, TNF, IFNg, IL13, IL4, Jnk, PRF1, STAT5, PRKCQ, IL2 receptor Beta, SOCS2, MYD88, STAT3, STAT1, TBX21, LCK, JAK3, IL& receptor, ABL1, IL9, STAT5A, STAT5B, Tcf7, PRDM1, and/or EOMES.

In several embodiments, gene editing of the immune cells can also provide unexpected enhancement in the expansion, persistence and/or cytotoxicity of the edited immune cell. As disclosed herein, engineered cells (e.g., those expressing a CAR) may also be edited, the combination of which provides for a robust cell for immunotherapy. In several embodiments, the edits allow for unexpectedly improved NK cell expansion, persistence and/or cytotoxicity. In several embodiments, knockout of CISH expression in NK cells removes a potent negative regulator of IL15-mediated signaling in NK cells, disinhibits the NK cells and allows for one or more of enhanced NK cell homing, NK cell migration, activation of NK cells, expansion, cytotoxicity and/or persistence. Additionally, in several embodiments, the editing can enhance NK and/or T cell function in the otherwise suppressive tumor microenvironment. In several embodiments, CISH gene editing results in enhanced NK cell expansion, persistence and/or cytotoxicity without requiring Notch ligand being provided exogenously. Additional

In several embodiments, gene editing is accomplished by one or more of a variety of engineered nucleases. In several embodiments, restriction enzymes are used, particularly when double strand breaks are desired at multiple regions. In several embodiments, a bioengineered nuclease is used. Depending on the embodiment, one or more of a Zinc Finger Nuclease (ZFN), transcription-activator like effector nuclease (TALEN), meganuclease and/or clustered regularly interspaced short palindromic repeats (CRISPR/Cas9) system are used to specifically edit the genes encoding one or more of the TCR subunits.

Meganucleases are characterized by their capacity to recognize and cut large DNA sequences (from 14 to 40 base pairs). In several embodiments, a meganuclease from the LAGLIDADG family is used, and is subjected to mutagenesis and screening to generate a meganuclease variant that recognizes a unique sequence(s), such as a specific site in the TCR, or CISH, or any other target gene disclosed herein. Target sites in the TCR can readily be identified. Further information of target sites within a region of the TCR can be found in US Patent Publication No. 2018/0325955, and US Patent Publication No. 2015/0017136, each of which is incorporated by reference herein in its entirety. In several embodiments, two or more meganucleases, or functions fragments thereof, are fused to create a hybrid enzymes that recognize a desired target sequence within the target gene (e.g., CISH).

In contrast to meganucleases, ZFNs and TALEN function based on a non-specific DNA cutting catalytic domain which is linked to specific DNA sequence recognizing peptides such as zinc fingers or transcription activator-like effectors (TALEs). Advantageously, the ZFNs and TALENs thus allow sequence-independent cleavage of DNA, with a high degree of sequence-specificity in target recognition. Zinc finger motifs naturally function in transcription factors to recognize specific DNA sequences for transcription. The C-terminal part of each finger is responsible for the specific recognition of the DNA sequence. While the sequences recognized by ZFNs are relatively short, (e.g., ˜3 base pairs), in several embodiments, combinations of 2, 3, 4, 5, 6, 7, 8, 9, 10 or more zinc fingers whose recognition sites have been characterized are used, thereby allowing targeting of specific sequences, such as a portion of the TCR (or an immune checkpoint inhibitor). The combined ZFNs are then fused with the catalytic domain(s) of an endonuclease, such as FokI (optionally a FokI heterodimer), in order to induce a targeted DNA break. Additional information on uses of ZFNs to edit the TCR and/or immune checkpoint inhibitors can be found in U.S. Pat. No. 9,597,357, which is incorporated by reference herein.

Transcription activator-like effector nucleases (TALENs) are specific DNA-binding proteins that feature an array of 33 or 34-amino acid repeats. Like ZFNs, TALENs are a fusion of a DNA cutting domain of a nuclease to TALE domains, which allow for sequence-independent introduction of double stranded DNA breaks with highly precise target site recognition. TALENs can create double strand breaks at the target site that can be repaired by error-prone non-homologous end-joining (NHEJ), resulting in gene disruptions through the introduction of small insertions or deletions. Advantageously, TALENs are used in several embodiments, at least in part due to their higher specificity in DNA binding, reduced off-target effects, and ease in construction of the DNA-binding domain.

CRISPRs (Clustered Regularly Interspaced Short Palindromic Repeats) are genetic elements that bacteria use as protection against viruses. The repeats are short sequences that originate from viral genomes and have been incorporated into the bacterial genome. Cas (CRISPR associated proteins) process these sequences and cut matching viral DNA sequences. By introducing plasmids containing Cas genes and specifically constructed CRISPRs into eukaryotic cells, the eukaryotic genome can be cut at any desired position. Additional information on CRISPR can be found in US Patent Publication No. 2014/0068797, which is incorporated by reference herein. In several embodiments, CRISPR is used to manipulate the gene(s) encoding a target gene to be knocked out or knocked in, for example CISH, TGFBR2, TCR, B2M, CIITA, CD47, HLA-E, etc. In several embodiments, CRISPR is used to edit one or more of the TCRs of a T cell and/or the genes encoding one or more immune checkpoint inhibitors. In several embodiments, the immune checkpoint inhibitor is selected from one or more of CTLA4 and PD1. In several embodiments, CRISPR is used to truncate one or more of TCRα, TCRβ, TCRγ, and TCRS. In several embodiments, a TCR is truncated without impacting the function of the CD3z signaling domain of the TCR. Depending on the embodiment and which target gene is to be edited, a Class 1 or Class 2 Cas is used. In several embodiments, a Class 1 Cas is used and the Cas type is selected from the following types: I, IA, IB, IC, ID, IE, IF, IU, III, IIIA, IIIB, IIIC, IIID, IV IVA, IVB, and combinations thereof. In several embodiments, the Cas is selected from the group consisting of Cas3, Cas8a, Cas5, Cas8b, Cas8c, Cas10d, Cse1, Cse2, Csy1, Csy2, Csy3, GSU0054, Cas10, Csm2, Cmr5, Cas10, Csx11, Csx10, Csf1, and combinations thereof. In several embodiments, a Class 2 Cas is used and the Cas type is selected from the following types: II, IIA, IIB, IIC, V, VI, and combinations thereof. In several embodiments, the Cas is selected from the group consisting of Cas9, Csn2, Cas4, Cpf1, C2c1, C2c3, Cas13a (previously known as C2c2), Cas13b, Cas13c, CasX, CasY and combinations thereof. In some embodiments, class 2 CasX is used, wherein CasX is capable of forming a complex with a guide nucleic acid and wherein the complex can bind to a target DNA, and wherein the target DNA comprises a non-target strand and a target strand. In some embodiments, class 2 CasY is used, wherein CasY is capable of binding and modifying a target nucleic acid and/or a polypeptide associated with target nucleic acid.

### Extracellular Domains (Binding Moieties)

Some embodiments of the compositions and methods described herein relate to a chimeric antigen receptor, such as a BCMA-directed chimeric antigen receptor, comprising an extracellular domain. In some embodiments, the extracellular domain comprises a binding moiety (also referred to as an antigen-binding protein or antigen-binding domain) that may be targeted to a tumor antigen, as described herein. in some embodiments, the binding moiety is derived from or comprises wild-type or non-wild-type sequence of an antibody, an antibody fragment, an scFv, a Fv, a Fab, a (Fab′)2, a single domain antibody (sdAb), a VH or VL domain, a camelid VHH domain, or a non-immunoglobulin scaffold such as a DARPIN, an affibody, an affilin, an adnectin, an affitin, a repebody, a fynomer, an alphabody, an avimer, an atrimer, a centyrin, a pronectin, an anticalin, a kunitz domain, an Armadillo repeat protein, an autoantigen, a receptor or a ligand. In some embodiments, the binding moiety contains more than one antigen binding domain. In embodiments, the binding moiety is operably linked directly or via an optional linker to the NH2-terminal end of a TCR domain (e.g. constant chains of TCR-alpha, TCR-beta1, TCR-beta2, preTCR-alpha, pre-TCR-alpha-Del48, TCR-gamma, or TCR-delta).

There are provided, in several embodiments, binding moieties (also referred to as antigen-binding proteins). As used herein, the term “binding moiety” shall be given its ordinary meaning, and shall also refer to a protein comprising an antigen-binding fragment that binds to an antigen and, optionally, a scaffold or framework portion that allows the antigen-binding fragment to adopt a conformation that promotes binding of the binding moiety to the antigen. In some embodiments, the antigen is a cancer antigen (e.g., BCMA) or a fragment thereof. In some embodiments, the antigen-binding fragment comprises at least one CDR from an antibody that binds to the antigen. In some embodiments, the antigen-binding fragment comprises all three CDRs from the heavy chain of an antibody that binds to the antigen or from the light chain of an antibody that binds to the antigen. In still some embodiments, the antigen-binding fragment comprises all six CDRs from an antibody that binds to the antigen (three from the heavy chain and three from the light chain). In several embodiments, the antigen-binding fragment comprises one, two, three, four, five, or six CDRs from an antibody that binds to the antigen, and in several embodiments, the CDRs can be any combination of heavy and/or light chain CDRs. The antigen-binding fragment in some embodiments is an antibody fragment.

Nonlimiting examples of binding moieties include antibodies, antibody fragments (e.g., an antigen-binding fragment of an antibody), antibody derivatives, and antibody analogs. Further specific examples include, but are not limited to, a single-chain variable fragment (scFv), a nanobody (e.g. VH domain of camelid heavy chain antibodies; VHH fragment), an Fab fragment, an Fab′ fragment, an F(ab′)2 fragment, an Fv fragment, an Fd fragment, and a complementarity determining region (CDR) fragment. These molecules can be derived from any mammalian source, such as human, mouse, rat, rabbit, or pig, dog, or camelid. Antibody fragments may compete for binding of a target antigen with an intact (e.g., native) antibody and the fragments may be produced by the modification of intact antibodies (e.g. enzymatic or chemical cleavage) or synthesized de novo using recombinant DNA technologies or peptide synthesis. The binding moiety can comprise, for example, an alternative protein scaffold or artificial scaffold with grafted CDRs or CDR derivatives. Such scaffolds include, but are not limited to, antibody-derived scaffolds comprising mutations introduced to, for example, stabilize the three-dimensional structure of the binding moiety as well as wholly synthetic scaffolds comprising, for example, a biocompatible polymer. In addition, peptide antibody mimetics (“PAMs”) can be used, as well as scaffolds based on antibody mimetics utilizing fibronectin components as a scaffold.

In some embodiments, the binding moiety comprises one or more antibody fragments incorporated into a single polypeptide chain or into multiple polypeptide chains. For instance, binding moieties can include, but are not limited to, a diabody; an intrabody; a domain antibody (single VL or VH domain or two or more VH domains joined by a peptide linker); a maxibody (2 scFvs fused to Fc region); a triabody; a tetrabody; a minibody (scFv fused to CH3 domain); a peptibody (one or more peptides attached to an Fc region); a linear antibody (a pair of tandem Fd segments (VH-CH1-VH-CH1) which, together with complementary light chain polypeptides, form a pair of antigen binding regions); a small modular immunopharmaceutical; and immunoglobulin fusion proteins (e.g. IgG-scFv, IgG-Fab, 2scFv-IgG, 4scFv-IgG, VH-IgG, IgG-VH, and Fab-scFv-Fc).

In some embodiments, the binding moiety has the structure of an immunoglobulin. As used herein, the term “immunoglobulin” shall be given its ordinary meaning, and shall also refer to a tetrameric molecule, with each tetramer comprising two identical pairs of polypeptide chains, each pair having one “light” (about 25 kDa) and one “heavy” chain (about 50-70 kDa). The amino-terminal portion of each chain includes a variable region of about 100 to 110 or more amino acids primarily responsible for antigen recognition. The carboxy-terminal portion of each chain defines a constant region primarily responsible for effector function.

Within light and heavy chains, the variable (V) and constant regions (C) are joined by a “J” region of about 12 or more amino acids, with the heavy chain also including a “D” region of about 10 more amino acids. The variable regions of each light/heavy chain pair form the antibody binding site such that an intact immunoglobulin has two binding sites.

Immunoglobulin chains exhibit the same general structure of relatively conserved framework regions (FR) joined by three hypervariable regions, also called complementarity determining regions or CDRs. From N-terminus to C-terminus, both light and heavy chains comprise the domains FR1, CDR1, FR2, CDR2, FR3, CDR3 and FR4.

Human light chains are classified as kappa and lambda light chains. An antibody “light chain”, refers to the smaller of the two types of polypeptide chains present in antibody molecules in their naturally occurring conformations. Kappa (K) and lambda (k) light chains refer to the two major antibody light chain isotypes. A light chain may include a polypeptide comprising, from amino terminus to carboxyl terminus, a single immunoglobulin light chain variable region (VL) and a single immunoglobulin light chain constant domain (CL).

Heavy chains are classified as mu (μ), delta (Δ), gamma (γ), alpha (α), and epsilon (ε), and define the antibody's isotype as IgM, IgD, IgG, IgA, and IgE, respectively. An antibody “heavy chain” refers to the larger of the two types of polypeptide chains present in antibody molecules in their naturally occurring conformations, and which normally determines the class to which the antibody belongs. A heavy chain may include a polypeptide comprising, from amino terminus to carboxyl terminus, a single immunoglobulin heavy chain variable region (VH), an immunoglobulin heavy chain constant domain 1 (CH1), an immunoglobulin hinge region, an immunoglobulin heavy chain constant domain 2 (CH2), an immunoglobulin heavy chain constant domain 3 (CH3), and optionally an immunoglobulin heavy chain constant domain 4 (CH4).

The IgG-class is further divided into subclasses, namely, IgG1, IgG2, IgG3, and IgG4. The IgA-class is further divided into subclasses, namely IgA1 and IgA2. The IgM has subclasses including, but not limited to, IgM1 and IgM2. The heavy chains in IgG, IgA, and IgD antibodies have three domains (CH1, CH2, and CH3), whereas the heavy chains in IgM and IgE antibodies have four domains (CH1, CH2, CH3, and CH4). The immunoglobulin heavy chain constant domains can be from any immunoglobulin isotype, including subtypes. The antibody chains are linked together via inter-polypeptide disulfide bonds between the CL domain and the CH1 domain (e.g., between the light and heavy chain) and between the hinge regions of the antibody heavy chains.

In some embodiments, the binding moiety is an antibody. The term “antibody”, as used herein, refers to a protein, or polypeptide sequence derived from an immunoglobulin molecule which specifically binds with an antigen. Antibodies can be monoclonal, or polyclonal, multiple or single chain, or intact immunoglobulins, and may be derived from natural sources or from recombinant sources. Antibodies can be tetramers of immunoglobulin molecules. The antibody may be “humanized”, “chimeric” or non-human. An antibody may include an intact immunoglobulin of any isotype, and includes, for instance, chimeric, humanized, human, and bispecific antibodies. An intact antibody will generally comprise at least two full-length heavy chains and two full-length light chains. Antibody sequences can be derived solely from a single species, or can be “chimeric,” that is, different portions of the antibody can be derived from two different species as described further below. Unless otherwise indicated, the term “antibody” also includes antibodies comprising two substantially full-length heavy chains and two substantially full-length light chains provided the antibodies retain the same or similar binding and/or function as the antibody comprised of two full length light and heavy chains. For example, antibodies having 1, 2, 3, 4, or 5 amino acid residue substitutions, insertions or deletions at the N-terminus and/or C-terminus of the heavy and/or light chains are included in the definition provided that the antibodies retain the same or similar binding and/or function as the antibodies comprising two full length heavy chains and two full length light chains. Examples of antibodies include monoclonal antibodies, polyclonal antibodies, chimeric antibodies, humanized antibodies, human antibodies, bispecific antibodies, and synthetic antibodies. There is provided, in some embodiments, monoclonal and polyclonal antibodies. As used herein, the term “polyclonal antibody” shall be given its ordinary meaning, and shall also refer to a population of antibodies that are typically widely varied in composition and binding specificity. As used herein, the term “monoclonal antibody” (“mAb”) shall be given its ordinary meaning, and shall also refer to one or more of a population of antibodies having identical sequences. Monoclonal antibodies bind to the antigen at a particular epitope on the antigen.

In some embodiments, the binding moiety is a fragment or antigen-binding fragment of an antibody. The term “antibody fragment” refers to at least one portion of an antibody, that retains the ability to specifically interact with (e.g., by binding, steric hindrance, stabilizing/destabilizing, spatial distribution) an epitope of an antigen. Examples of antibody fragments include, but are not limited to, Fab, Fab′, F(ab′)2, Fv fragments, scFv antibody fragments, disulfide-linked Fvs (sdFv), a Fd fragment consisting of the VH and CHI domains, linear antibodies, single domain antibodies such as sdAb (either VL or VH), camelid VHH domains, multi-specific antibodies formed from antibody fragments such as a bivalent fragment comprising two Fab fragments linked by a disulfide bridge at the hinge region, and an isolated CDR or other epitope binding fragments of an antibody. An antigen binding fragment can also be incorporated into single domain antibodies, maxibodies, minibodies, nanobodies, intrabodies, diabodies, triabodies, tetrabodies, v-NAR and bis-scFv (see, e.g., Hollinger and Hudson, Nature Biotechnology 23: 1126-1136, 2005). Antigen binding fragments can also be grafted into scaffolds based on polypeptides such as a fibronectin type III (Fn3) (see U.S. Pat. No. 6,703,199, which describes fibronectin polypeptide mini bodies). An antibody fragment may include a Fab, Fab′, F(ab′)2, and/or Fv fragment that contains at least one CDR of an immunoglobulin that is sufficient to confer specific antigen binding to a cancer antigen. Antibody fragments may be produced by recombinant DNA techniques or by enzymatic or chemical cleavage of intact antibodies.

In some embodiments, Fab fragments are provided. A Fab fragment is a monovalent fragment having the VL, VH, CL and CH1 domains; a F(ab′)2 fragment is a bivalent fragment having two Fab fragments linked by a disulfide bridge at the hinge region; a Fd fragment has the VH and CH1 domains; an Fv fragment has the VL and VH domains of a single arm of an antibody; and a dAb fragment has a VH domain, a VL domain, or an antigen-binding fragment of a VH or VL domain. In some embodiments, these antibody fragments can be incorporated into single domain antibodies, single-chain antibodies, maxibodies, minibodies, intrabodies, diabodies, triabodies, tetrabodies, v-NAR and bis-scFv. In some embodiments, the antibodies comprise at least one CDR as described herein.

There is also provided for herein, in several embodiments, single-chain variable fragments. As used herein, the term “single-chain variable fragment” (“scFv”) shall be given its ordinary meaning, and shall also refer to a fusion protein in which a VL and a VH region are joined via a linker (e.g., a synthetic sequence of amino acid residues) to form a continuous protein chain wherein the linker is long enough to allow the protein chain to fold back on itself and form a monovalent antigen binding site). For the sake of clarity, unless otherwise indicated as such, a “single-chain variable fragment” is not an antibody as defined herein. Diabodies are bivalent antibodies comprising two polypeptide chains, wherein each polypeptide chain comprises VH and VL domains joined by a linker that is configured to reduce or not allow for pairing between two domains on the same chain, thus allowing each domain to pair with a complementary domain on another polypeptide chain. According to several embodiments, if the two polypeptide chains of a diabody are identical, then a diabody resulting from their pairing will have two identical antigen binding sites. Polypeptide chains having different sequences can be used to make a diabody with two different antigen binding sites. Similarly, tribodies and tetrabodies are antibodies comprising three and four polypeptide chains, respectively, and forming three and four antigen binding sites, respectively, which can be the same or different.

In several embodiments, the binding moiety comprises one or more CDRs. As used herein, the term “CDR” shall be given its ordinary meaning, and shall also refer to the complementarity determining region (also termed “minimal recognition units” or “hypervariable region”) within antibody variable sequences. The CDRs permit the binding moiety to specifically bind to a particular antigen of interest. There are three heavy chain variable region CDRs (HCDR1, HCDR2 and HCDR3) and three light chain variable region CDRs (LCDR1, LCDR2 and LCDR3). The CDRs in each of the two chains typically are aligned by the framework regions to form a structure that binds specifically to a specific epitope or domain on the target protein. From N-terminus to C-terminus, naturally-occurring light and heavy chain variable regions both typically conform to the following order of these elements: FR1, CDR1, FR2, CDR2, FR3, CDR3 and FR4. A numbering system has been devised for assigning numbers to amino acids that occupy positions in each of these domains. This numbering system is defined in Kabat Sequences of Proteins of Immunological Interest (1987 and 1991, NIH, Bethesda, MD), or Chothia & Lesk, 1987, J. Mol. Biol. 196:901-917; Chothia et al., 1989, Nature 342:878-883. Complementarity determining regions (CDRs) and framework regions (FR) of a given antibody may be identified using this system. Other numbering systems for the amino acids in immunoglobulin chains include IMGT® (the international ImMunoGeneTics information system; Lefranc et al, Dev. Comp. Immunol. 29:185-203; 2005) and AHo (Honegger and Pluckthun, J. Mol. Biol. 309(3):657-670; 2001). One or more CDRs may be incorporated into a molecule either covalently or noncovalently to make it an binding moiety.

### Anti-BCMA Binding Moieties

Disclosed herein are anti-BCMA binding moieties comprising a heavy chain variable region (VH) comprising heavy chain complementarity determining regions (CDRs) 1, 2, and 3 (HCDR1, HCDR2, and HCDR3), and a light chain variable region (VL) comprising light chain CDRs 1, 2, and 3 (LCDR1, LCDR2, and LCDR3).

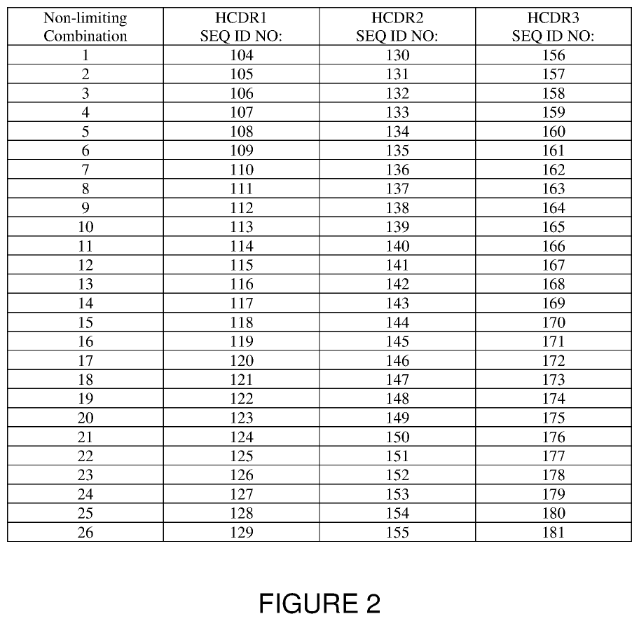

In some embodiments, the HCDR1 comprises a sequence having at least 85%, 90%, 91%, 92%, 93%, 94%, 95%, 96%, 97%, 98%, 99%, or 100% identity to any one of SEQ ID NOs: 104-129. In some embodiments, the HCDR2 comprises a sequence having at least 85%, 90%, 91%, 92%, 93%, 94%, 95%, 96%, 97%, 98%, 99%, or 100% identity to any one of SEQ ID NOs: 130-155. In some embodiments, the HCDR3 comprises a sequence having at least 85%, 90%, 91%, 92%, 93%, 94%, 95%, 96%, 97%, 98%, 99%, or 100% identity to any one of SEQ ID NOs: 156-181. In some embodiments, the HCDR1 comprises a sequence having at least 85%, 90%, 91%, 92%, 93%, 94%, 95%, 96%, 97%, 98%, 99%, or 100% identity to any one of SEQ ID NOs: 104-129, the HCDR2 comprises a sequence having at least 85%, 90%, 91%, 92%, 93%, 94%, 95%, 96%, 97%, 98%, 99%, or 100% identity to any one of SEQ ID NOs: 130-155, and the HCDR3 comprises a sequence having at least 85%, 90%, 91%, 92%, 93%, 94%, 95%, 96%, 97%, 98%, 99%, or 100% identity to any one of SEQ ID NOs: 156-181. In some embodiments, the VH comprises a combination of HCDR1, HCDR2, and HCDR3 as depicted in FIG. 2.

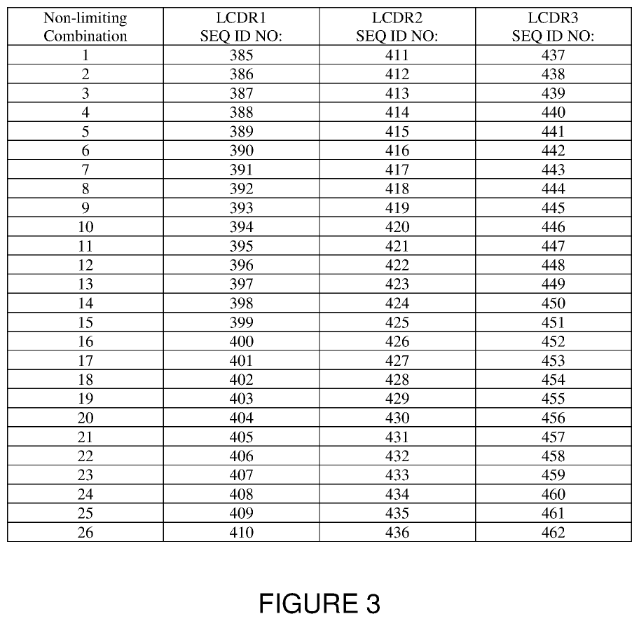

In some embodiments, the LCDR1 comprises a sequence having at least 85%, 90%, 91%, 92%, 93%, 94%, 95%, 96%, 97%, 98%, 99%, or 100% identity to any one of SEQ ID NOs: 385-410. In some embodiments, the LCDR2 comprises a sequence having at least 85%, 90%, 91%, 92%, 93%, 94%, 95%, 96%, 97%, 98%, 99%, or 100% identity to any one of SEQ ID NOs: 411-436. In some embodiments, the LCDR3 comprises a sequence having at least 85%, 90%, 91%, 92%, 93%, 94%, 95%, 96%, 97%, 98%, 99%, or 100% identity to any one of SEQ ID NOs: 437-462. In some embodiments, the LCDR1 comprises a sequence having at least 85%, 90%, 91%, 92%, 93%, 94%, 95%, 96%, 97%, 98%, 99%, or 100% identity to any one of SEQ ID NOs: 385-410, the LCDR2 comprises a sequence having at least 85%, 90%, 91%, 92%, 93%, 94%, 95%, 96%, 97%, 98%, 99%, or 100% identity to any one of SEQ ID NOs: 411-436, and the LCDR3 comprises a sequence having at least 85%, 90%, 91%, 92%, 93%, 94%, 95%, 96%, 97%, 98%, 99%, or 100% identity to any one of SEQ ID NOs: 437-462. In some embodiments, the VL comprises a combination of LCDR1, LCDR2, and LCDR3 as depicted in FIG. 3.

In some embodiments, 1) the HCDR1 comprises a sequence having at least 85%, 90%, 91%, 92%, 93%, 94%, 95%, 96%, 97%, 98%, 99%, or 100% identity to SEQ ID NO: 104, the HCDR2 comprises a sequence having at least 85%, 90%, 91%, 92%, 93%, 94%, 95%, 96%, 97%, 98%, 99%, or 100% identity to SEQ ID NO: 130, and the HCDR3 comprises a sequence having at least 85%, 90%, 91%, 92%, 93%, 94%, 95%, 96%, 97%, 98%, 99%, or 100% identity to SEQ ID NO: 156;
2) the HCDR1 comprises a sequence having at least 85%, 90%, 91%, 92%, 93%, 94%, 95%, 96%, 97%, 98%, 99%, or 100% identity to SEQ ID NO: 105, the HCDR2 comprises a sequence having at least 85%, 90%, 91%, 92%, 93%, 94%, 95%, 96%, 97%, 98%, 99%, or 100% identity to SEQ ID NO: 131, and the HCDR3 comprises a sequence having at least 85%, 90%, 91%, 92%, 93%, 94%, 95%, 96%, 97%, 98%, 99%, or 100% identity to SEQ ID NO: 157;
3) the HCDR1 comprises a sequence having at least 85%, 90%, 91%, 92%, 93%, 94%, 95%, 96%, 97%, 98%, 99%, or 100% identity to SEQ ID NO: 106, the HCDR2 comprises a sequence having at least 85%, 90%, 91%, 92%, 93%, 94%, 95%, 96%, 97%, 98%, 99%, or 100% identity to SEQ ID NO: 132, and the HCDR3 comprises a sequence having at least 85%, 90%, 91%, 92%, 93%, 94%, 95%, 96%, 97%, 98%, 99%, or 100% identity to SEQ ID NO: 158;
4) the HCDR1 comprises a sequence having at least 85%, 90%, 91%, 92%, 93%, 94%, 95%, 96%, 97%, 98%, 99%, or 100% identity to SEQ ID NO: 107, the HCDR2 comprises a sequence having at least 85%, 90%, 91%, 92%, 93%, 94%, 95%, 96%, 97%, 98%, 99%, or 100% identity to SEQ ID NO: 133, and the HCDR3 comprises a sequence having at least 85%, 90%, 91%, 92%, 93%, 94%, 95%, 96%, 97%, 98%, 99%, or 100% identity to SEQ ID NO: 159;
5) the HCDR1 comprises a sequence having at least 85%, 90%, 91%, 92%, 93%, 94%, 95%, 96%, 97%, 98%, 99%, or 100% identity to SEQ ID NO: 108, the HCDR2 comprises a sequence having at least 85%, 90%, 91%, 92%, 93%, 94%, 95%, 96%, 97%, 98%, 99%, or 100% identity to SEQ ID NO: 134, and the HCDR3 comprises a sequence having at least 85%, 90%, 91%, 92%, 93%, 94%, 95%, 96%, 97%, 98%, 99%, or 100% identity to SEQ ID NO: 160;
6) the HCDR1 comprises a sequence having at least 85%, 90%, 91%, 92%, 93%, 94%, 95%, 96%, 97%, 98%, 99%, or 100% identity to SEQ ID NO: 109, the HCDR2 comprises a sequence having at least 85%, 90%, 91%, 92%, 93%, 94%, 95%, 96%, 97%, 98%, 99%, or 100% identity to SEQ ID NO: 135, and the HCDR3 comprises a sequence having at least 85%, 90%, 91%, 92%, 93%, 94%, 95%, 96%, 97%, 98%, 99%, or 100% identity to SEQ ID NO: 161;
7) the HCDR1 comprises a sequence having at least 85%, 90%, 91%, 92%, 93%, 94%, 95%, 96%, 97%, 98%, 99%, or 100% identity to SEQ ID NO: 110, the HCDR2 comprises a sequence having at least 85%, 90%, 91%, 92%, 93%, 94%, 95%, 96%, 97%, 98%, 99%, or 100% identity to SEQ ID NO: 136, and the HCDR3 comprises a sequence having at least 85%, 90%, 91%, 92%, 93%, 94%, 95%, 96%, 97%, 98%, 99%, or 100% identity to SEQ ID NO: 162;
8) the HCDR1 comprises a sequence having at least 85%, 90%, 91%, 92%, 93%, 94%, 95%, 96%, 97%, 98%, 99%, or 100% identity to SEQ ID NO: 111, the HCDR2 comprises a sequence having at least 85%, 90%, 91%, 92%, 93%, 94%, 95%, 96%, 97%, 98%, 99%, or 100% identity to SEQ ID NO: 137, and the HCDR3 comprises a sequence having at least 85%, 90%, 91%, 92%, 93%, 94%, 95%, 96%, 97%, 98%, 99%, or 100% identity to SEQ ID NO: 163;
9) the HCDR1 comprises a sequence having at least 85%, 90%, 91%, 92%, 93%, 94%, 95%, 96%, 97%, 98%, 99%, or 100% identity to SEQ ID NO: 112, the HCDR2 comprises a sequence having at least 85%, 90%, 91%, 92%, 93%, 94%, 95%, 96%, 97%, 98%, 99%, or 100% identity to SEQ ID NO: 138, and the HCDR3 comprises a sequence having at least 85%, 90%, 91%, 92%, 93%, 94%, 95%, 96%, 97%, 98%, 99%, or 100% identity to SEQ ID NO: 164;
10) the HCDR1 comprises a sequence having at least 85%, 90%, 91%, 92%, 93%, 94%, 95%, 96%, 97%, 98%, 99%, or 100% identity to SEQ ID NO: 113, the HCDR2 comprises a sequence having at least 85%, 90%, 91%, 92%, 93%, 94%, 95%, 96%, 97%, 98%, 99%, or 100% identity to SEQ ID NO: 139, and the HCDR3 comprises a sequence having at least 85%, 90%, 91%, 92%, 93%, 94%, 95%, 96%, 97%, 98%, 99%, or 100% identity to SEQ ID NO: 165;
11) the HCDR1 comprises a sequence having at least 85%, 90%, 91%, 92%, 93%, 94%, 95%, 96%, 97%, 98%, 99%, or 100% identity to SEQ ID NO: 114, the HCDR2 comprises a sequence having at least 85%, 90%, 91%, 92%, 93%, 94%, 95%, 96%, 97%, 98%, 99%, or 100% identity to SEQ ID NO: 140, and the HCDR3 comprises a sequence having at least 85%, 90%, 91%, 92%, 93%, 94%, 95%, 96%, 97%, 98%, 99%, or 100% identity to SEQ ID NO: 166;
12) the HCDR1 comprises a sequence having at least 85%, 90%, 91%, 92%, 93%, 94%, 95%, 96%, 97%, 98%, 99%, or 100% identity to SEQ ID NO: 115, the HCDR2 comprises a sequence having at least 85%, 90%, 91%, 92%, 93%, 94%, 95%, 96%, 97%, 98%, 99%, or 100% identity to SEQ ID NO: 141, and the HCDR3 comprises a sequence having at least 85%, 90%, 91%, 92%, 93%, 94%, 95%, 96%, 97%, 98%, 99%, or 100% identity to SEQ ID NO: 167;
13) the HCDR1 comprises a sequence having at least 85%, 90%, 91%, 92%, 93%, 94%, 95%, 96%, 97%, 98%, 99%, or 100% identity to SEQ ID NO: 116, the HCDR2 comprises a sequence having at least 85%, 90%, 91%, 92%, 93%, 94%, 95%, 96%, 97%, 98%, 99%, or 100% identity to SEQ ID NO: 142, and the HCDR3 comprises a sequence having at least 85%, 90%, 91%, 92%, 93%, 94%, 95%, 96%, 97%, 98%, 99%, or 100% identity to SEQ ID NO: 168;
14) the HCDR1 comprises a sequence having at least 85%, 90%, 91%, 92%, 93%, 94%, 95%, 96%, 97%, 98%, 99%, or 100% identity to SEQ ID NO: 117, the HCDR2 comprises a sequence having at least 85%, 90%, 91%, 92%, 93%, 94%, 95%, 96%, 97%, 98%, 99%, or 100% identity to SEQ ID NO: 143, and the HCDR3 comprises a sequence having at least 85%, 90%, 91%, 92%, 93%, 94%, 95%, 96%, 97%, 98%, 99%, or 100% identity to SEQ ID NO: 169;
15) the HCDR1 comprises a sequence having at least 85%, 90%, 91%, 92%, 93%, 94%, 95%, 96%, 97%, 98%, 99%, or 100% identity to SEQ ID NO: 118, the HCDR2 comprises a sequence having at least 85%, 90%, 91%, 92%, 93%, 94%, 95%, 96%, 97%, 98%, 99%, or 100% identity to SEQ ID NO: 144, and the HCDR3 comprises a sequence having at least 85%, 90%, 91%, 92%, 93%, 94%, 95%, 96%, 97%, 98%, 99%, or 100% identity to SEQ ID NO: 170;
16) the HCDR1 comprises a sequence having at least 85%, 90%, 91%, 92%, 93%, 94%, 95%, 96%, 97%, 98%, 99%, or 100% identity to SEQ ID NO: 119, the HCDR2 comprises a sequence having at least 85%, 90%, 91%, 92%, 93%, 94%, 95%, 96%, 97%, 98%, 99%, or 100% identity to SEQ ID NO: 145, and the HCDR3 comprises a sequence having at least 85%, 90%, 91%, 92%, 93%, 94%, 95%, 96%, 97%, 98%, 99%, or 100% identity to SEQ ID NO: 171;
17) the HCDR1 comprises a sequence having at least 85%, 90%, 91%, 92%, 93%, 94%, 95%, 96%, 97%, 98%, 99%, or 100% identity to SEQ ID NO: 120, the HCDR2 comprises a sequence having at least 85%, 90%, 91%, 92%, 93%, 94%, 95%, 96%, 97%, 98%, 99%, or 100% identity to SEQ ID NO: 146, and the HCDR3 comprises a sequence having at least 85%, 90%, 91%, 92%, 93%, 94%, 95%, 96%, 97%, 98%, 99%, or 100% identity to SEQ ID NO: 172;
18) the HCDR1 comprises a sequence having at least 85%, 90%, 91%, 92%, 93%, 94%, 95%, 96%, 97%, 98%, 99%, or 100% identity to SEQ ID NO: 121, the HCDR2 comprises a sequence having at least 85%, 90%, 91%, 92%, 93%, 94%, 95%, 96%, 97%, 98%, 99%, or 100% identity to SEQ ID NO: 147, and the HCDR3 comprises a sequence having at least 85%, 90%, 91%, 92%, 93%, 94%, 95%, 96%, 97%, 98%, 99%, or 100% identity to SEQ ID NO: 173;
19) the HCDR1 comprises a sequence having at least 85%, 90%, 91%, 92%, 93%, 94%, 95%, 96%, 97%, 98%, 99%, or 100% identity to SEQ ID NO: 122, the HCDR2 comprises a sequence having at least 85%, 90%, 91%, 92%, 93%, 94%, 95%, 96%, 97%, 98%, 99%, or 100% identity to SEQ ID NO: 148, and the HCDR3 comprises a sequence having at least 85%, 90%, 91%, 92%, 93%, 94%, 95%, 96%, 97%, 98%, 99%, or 100% identity to SEQ ID NO: 174;
20) the HCDR1 comprises a sequence having at least 85%, 90%, 91%, 92%, 93%, 94%, 95%, 96%, 97%, 98%, 99%, or 100% identity to SEQ ID NO: 123, the HCDR2 comprises a sequence having at least 85%, 90%, 91%, 92%, 93%, 94%, 95%, 96%, 97%, 98%, 99%, or 100% identity to SEQ ID NO: 149, and the HCDR3 comprises a sequence having at least 85%, 90%, 91%, 92%, 93%, 94%, 95%, 96%, 97%, 98%, 99%, or 100% identity to SEQ ID NO: 175;
21) the HCDR1 comprises a sequence having at least 85%, 90%, 91%, 92%, 93%, 94%, 95%, 96%, 97%, 98%, 99%, or 100% identity to SEQ ID NO: 124, the HCDR2 comprises a sequence having at least 85%, 90%, 91%, 92%, 93%, 94%, 95%, 96%, 97%, 98%, 99%, or 100% identity to SEQ ID NO: 150, and the HCDR3 comprises a sequence having at least 85%, 90%, 91%, 92%, 93%, 94%, 95%, 96%, 97%, 98%, 99%, or 100% identity to SEQ ID NO: 176;
22) the HCDR1 comprises a sequence having at least 85%, 90%, 91%, 92%, 93%, 94%, 95%, 96%, 97%, 98%, 99%, or 100% identity to SEQ ID NO: 125, the HCDR2 comprises a sequence having at least 85%, 90%, 91%, 92%, 93%, 94%, 95%, 96%, 97%, 98%, 99%, or 100% identity to SEQ ID NO: 151, and the HCDR3 comprises a sequence having at least 85%, 90%, 91%, 92%, 93%, 94%, 95%, 96%, 97%, 98%, 99%, or 100% identity to SEQ ID NO: 177;
23) the HCDR1 comprises a sequence having at least 85%, 90%, 91%, 92%, 93%, 94%, 95%, 96%, 97%, 98%, 99%, or 100% identity to SEQ ID NO: 126, the HCDR2 comprises a sequence having at least 85%, 90%, 91%, 92%, 93%, 94%, 95%, 96%, 97%, 98%, 99%, or 100% identity to SEQ ID NO: 152, and the HCDR3 comprises a sequence having at least 85%, 90%, 91%, 92%, 93%, 94%, 95%, 96%, 97%, 98%, 99%, or 100% identity to SEQ ID NO: 178;
24) the HCDR1 comprises a sequence having at least 85%, 90%, 91%, 92%, 93%, 94%, 95%, 96%, 97%, 98%, 99%, or 100% identity to SEQ ID NO: 127, the HCDR2 comprises a sequence having at least 85%, 90%, 91%, 92%, 93%, 94%, 95%, 96%, 97%, 98%, 99%, or 100% identity to SEQ ID NO: 153, and the HCDR3 comprises a sequence having at least 85%, 90%, 91%, 92%, 93%, 94%, 95%, 96%, 97%, 98%, 99%, or 100% identity to SEQ ID NO: 179;
25) the HCDR1 comprises a sequence having at least 85%, 90%, 91%, 92%, 93%, 94%, 95%, 96%, 97%, 98%, 99%, or 100% identity to SEQ ID NO: 128, the HCDR2 comprises a sequence having at least 85%, 90%, 91%, 92%, 93%, 94%, 95%, 96%, 97%, 98%, 99%, or 100% identity to SEQ ID NO: 154, and the HCDR3 comprises a sequence having at least 85%, 90%, 91%, 92%, 93%, 94%, 95%, 96%, 97%, 98%, 99%, or 100% identity to SEQ ID NO: 180; or
26) the HCDR1 comprises a sequence having at least 85%, 90%, 91%, 92%, 93%, 94%, 95%, 96%, 97%, 98%, 99%, or 100% identity to SEQ ID NO: 129, the HCDR2 comprises a sequence having at least 85%, 90%, 91%, 92%, 93%, 94%, 95%, 96%, 97%, 98%, 99%, or 100% identity to SEQ ID NO: 155, and the HCDR3 comprises a sequence having at least 85%, 90%, 91%, 92%, 93%, 94%, 95%, 96%, 97%, 98%, 99%, or 100% identity to SEQ ID NO: 181.

In some embodiments, 1) the LCDR1 comprises a sequence having at least 85%, 90%, 91%, 92%, 93%, 94%, 95%, 96%, 97%, 98%, 99%, or 100% identity to SEQ ID NO: 385, the LCDR2 comprises a sequence having at least 85%, 90%, 91%, 92%, 93%, 94%, 95%, 96%, 97%, 98%, 99%, or 100% identity to SEQ ID NO: 411, and the LCDR3 comprises a sequence having at least 85%, 90%, 91%, 92%, 93%, 94%, 95%, 96%, 97%, 98%, 99%, or 100% identity to SEQ ID NO: 437;
2) the LCDR1 comprises a sequence having at least 85%, 90%, 91%, 92%, 93%, 94%, 95%, 96%, 97%, 98%, 99%, or 100% identity to SEQ ID NO: 386, the LCDR2 comprises a sequence having at least 85%, 90%, 91%, 92%, 93%, 94%, 95%, 96%, 97%, 98%, 99%, or 100% identity to SEQ ID NO: 412, and the LCDR3 comprises a sequence having at least 85%, 90%, 91%, 92%, 93%, 94%, 95%, 96%, 97%, 98%, 99%, or 100% identity to SEQ ID NO: 438;
3) the LCDR1 comprises a sequence having at least 85%, 90%, 91%, 92%, 93%, 94%, 95%, 96%, 97%, 98%, 99%, or 100% identity to SEQ ID NO: 387, the LCDR2 comprises a sequence having at least 85%, 90%, 91%, 92%, 93%, 94%, 95%, 96%, 97%, 98%, 99%, or 100% identity to SEQ ID NO: 413, and the LCDR3 comprises a sequence having at least 85%, 90%, 91%, 92%, 93%, 94%, 95%, 96%, 97%, 98%, 99%, or 100% identity to SEQ ID NO: 439;
4) the LCDR1 comprises a sequence having at least 85%, 90%, 91%, 92%, 93%, 94%, 95%, 96%, 97%, 98%, 99%, or 100% identity to SEQ ID NO: 388, the LCDR2 comprises a sequence having at least 85%, 90%, 91%, 92%, 93%, 94%, 95%, 96%, 97%, 98%, 99%, or 100% identity to SEQ ID NO: 414, and the LCDR3 comprises a sequence having at least 85%, 90%, 91%, 92%, 93%, 94%, 95%, 96%, 97%, 98%, 99%, or 100% identity to SEQ ID NO: 440;
5) the LCDR1 comprises a sequence having at least 85%, 90%, 91%, 92%, 93%, 94%, 95%, 96%, 97%, 98%, 99%, or 100% identity to SEQ ID NO: 389, the LCDR2 comprises a sequence having at least 85%, 90%, 91%, 92%, 93%, 94%, 95%, 96%, 97%, 98%, 99%, or 100% identity to SEQ ID NO: 415, and the LCDR3 comprises a sequence having at least 85%, 90%, 91%, 92%, 93%, 94%, 95%, 96%, 97%, 98%, 99%, or 100% identity to SEQ ID NO: 441;
6) the LCDR1 comprises a sequence having at least 85%, 90%, 91%, 92%, 93%, 94%, 95%, 96%, 97%, 98%, 99%, or 100% identity to SEQ ID NO: 390, the LCDR2 comprises a sequence having at least 85%, 90%, 91%, 92%, 93%, 94%, 95%, 96%, 97%, 98%, 99%, or 100% identity to SEQ ID NO: 416, and the LCDR3 comprises a sequence having at least 85%, 90%, 91%, 92%, 93%, 94%, 95%, 96%, 97%, 98%, 99%, or 100% identity to SEQ ID NO: 442;
7) the LCDR1 comprises a sequence having at least 85%, 90%, 91%, 92%, 93%, 94%, 95%, 96%, 97%, 98%, 99%, or 100% identity to SEQ ID NO: 391, the LCDR2 comprises a sequence having at least 85%, 90%, 91%, 92%, 93%, 94%, 95%, 96%, 97%, 98%, 99%, or 100% identity to SEQ ID NO: 417, and the LCDR3 comprises a sequence having at least 85%, 90%, 91%, 92%, 93%, 94%, 95%, 96%, 97%, 98%, 99%, or 100% identity to SEQ ID NO: 443;
8) the LCDR1 comprises a sequence having at least 85%, 90%, 91%, 92%, 93%, 94%, 95%, 96%, 97%, 98%, 99%, or 100% identity to SEQ ID NO: 392, the LCDR2 comprises a sequence having at least 85%, 90%, 91%, 92%, 93%, 94%, 95%, 96%, 97%, 98%, 99%, or 100% identity to SEQ ID NO: 418, and the LCDR3 comprises a sequence having at least 85%, 90%, 91%, 92%, 93%, 94%, 95%, 96%, 97%, 98%, 99%, or 100% identity to SEQ ID NO: 444;
9) the LCDR1 comprises a sequence having at least 85%, 90%, 91%, 92%, 93%, 94%, 95%, 96%, 97%, 98%, 99%, or 100% identity to SEQ ID NO: 393, the LCDR2 comprises a sequence having at least 85%, 90%, 91%, 92%, 93%, 94%, 95%, 96%, 97%, 98%, 99%, or 100% identity to SEQ ID NO: 419, and the LCDR3 comprises a sequence having at least 85%, 90%, 91%, 92%, 93%, 94%, 95%, 96%, 97%, 98%, 99%, or 100% identity to SEQ ID NO: 445;
10) the LCDR1 comprises a sequence having at least 85%, 90%, 91%, 92%, 93%, 94%, 95%, 96%, 97%, 98%, 99%, or 100% identity to SEQ ID NO: 394, the LCDR2 comprises a sequence having at least 85%, 90%, 91%, 92%, 93%, 94%, 95%, 96%, 97%, 98%, 99%, or 100% identity to SEQ ID NO: 420, and the LCDR3 comprises a sequence having at least 85%, 90%, 91%, 92%, 93%, 94%, 95%, 96%, 97%, 98%, 99%, or 100% identity to SEQ ID NO: 446;
11) the LCDR1 comprises a sequence having at least 85%, 90%, 91%, 92%, 93%, 94%, 95%, 96%, 97%, 98%, 99%, or 100% identity to SEQ ID NO: 395, the LCDR2 comprises a sequence having at least 85%, 90%, 91%, 92%, 93%, 94%, 95%, 96%, 97%, 98%, 99%, or 100% identity to SEQ ID NO: 421, and the LCDR3 comprises a sequence having at least 85%, 90%, 91%, 92%, 93%, 94%, 95%, 96%, 97%, 98%, 99%, or 100% identity to SEQ ID NO: 447;
12) the LCDR1 comprises a sequence having at least 85%, 90%, 91%, 92%, 93%, 94%, 95%, 96%, 97%, 98%, 99%, or 100% identity to SEQ ID NO: 396, the LCDR2 comprises a sequence having at least 85%, 90%, 91%, 92%, 93%, 94%, 95%, 96%, 97%, 98%, 99%, or 100% identity to SEQ ID NO: 422, and the LCDR3 comprises a sequence having at least 85%, 90%, 91%, 92%, 93%, 94%, 95%, 96%, 97%, 98%, 99%, or 100% identity to SEQ ID NO: 448;
13) the LCDR1 comprises a sequence having at least 85%, 90%, 91%, 92%, 93%, 94%, 95%, 96%, 97%, 98%, 99%, or 100% identity to SEQ ID NO: 397, the LCDR2 comprises a sequence having at least 85%, 90%, 91%, 92%, 93%, 94%, 95%, 96%, 97%, 98%, 99%, or 100% identity to SEQ ID NO: 423, and the LCDR3 comprises a sequence having at least 85%, 90%, 91%, 92%, 93%, 94%, 95%, 96%, 97%, 98%, 99%, or 100% identity to SEQ ID NO: 449;
14) the LCDR1 comprises a sequence having at least 85%, 90%, 91%, 92%, 93%, 94%, 95%, 96%, 97%, 98%, 99%, or 100% identity to SEQ ID NO: 398, the LCDR2 comprises a sequence having at least 85%, 90%, 91%, 92%, 93%, 94%, 95%, 96%, 97%, 98%, 99%, or 100% identity to SEQ ID NO: 424, and the LCDR3 comprises a sequence having at least 85%, 90%, 91%, 92%, 93%, 94%, 95%, 96%, 97%, 98%, 99%, or 100% identity to SEQ ID NO: 450;
15) the LCDR1 comprises a sequence having at least 85%, 90%, 91%, 92%, 93%, 94%, 95%, 96%, 97%, 98%, 99%, or 100% identity to SEQ ID NO: 399, the LCDR2 comprises a sequence having at least 85%, 90%, 91%, 92%, 93%, 94%, 95%, 96%, 97%, 98%, 99%, or 100% identity to SEQ ID NO: 425, and the LCDR3 comprises a sequence having at least 85%, 90%, 91%, 92%, 93%, 94%, 95%, 96%, 97%, 98%, 99%, or 100% identity to SEQ ID NO: 451;
16) the LCDR1 comprises a sequence having at least 85%, 90%, 91%, 92%, 93%, 94%, 95%, 96%, 97%, 98%, 99%, or 100% identity to SEQ ID NO: 400, the LCDR2 comprises a sequence having at least 85%, 90%, 91%, 92%, 93%, 94%, 95%, 96%, 97%, 98%, 99%, or 100% identity to SEQ ID NO: 426, and the LCDR3 comprises a sequence having at least 85%, 90%, 91%, 92%, 93%, 94%, 95%, 96%, 97%, 98%, 99%, or 100% identity to SEQ ID NO: 452;
17) the LCDR1 comprises a sequence having at least 85%, 90%, 91%, 92%, 93%, 94%, 95%, 96%, 97%, 98%, 99%, or 100% identity to SEQ ID NO: 401, the LCDR2 comprises a sequence having at least 85%, 90%, 91%, 92%, 93%, 94%, 95%, 96%, 97%, 98%, 99%, or 100% identity to SEQ ID NO: 427, and the LCDR3 comprises a sequence having at least 85%, 90%, 91%, 92%, 93%, 94%, 95%, 96%, 97%, 98%, 99%, or 100% identity to SEQ ID NO: 453;
18) the LCDR1 comprises a sequence having at least 85%, 90%, 91%, 92%, 93%, 94%, 95%, 96%, 97%, 98%, 99%, or 100% identity to SEQ ID NO: 402, the LCDR2 comprises a sequence having at least 85%, 90%, 91%, 92%, 93%, 94%, 95%, 96%, 97%, 98%, 99%, or 100% identity to SEQ ID NO: 428, and the LCDR3 comprises a sequence having at least 85%, 90%, 91%, 92%, 93%, 94%, 95%, 96%, 97%, 98%, 99%, or 100% identity to SEQ ID NO: 454;
19) the LCDR1 comprises a sequence having at least 85%, 90%, 91%, 92%, 93%, 94%, 95%, 96%, 97%, 98%, 99%, or 100% identity to SEQ ID NO: 403, the LCDR2 comprises a sequence having at least 85%, 90%, 91%, 92%, 93%, 94%, 95%, 96%, 97%, 98%, 99%, or 100% identity to SEQ ID NO: 429, and the LCDR3 comprises a sequence having at least 85%, 90%, 91%, 92%, 93%, 94%, 95%, 96%, 97%, 98%, 99%, or 100% identity to SEQ ID NO: 455;
20) the LCDR1 comprises a sequence having at least 85%, 90%, 91%, 92%, 93%, 94%, 95%, 96%, 97%, 98%, 99%, or 100% identity to SEQ ID NO: 404, the LCDR2 comprises a sequence having at least 85%, 90%, 91%, 92%, 93%, 94%, 95%, 96%, 97%, 98%, 99%, or 100% identity to SEQ ID NO: 430, and the LCDR3 comprises a sequence having at least 85%, 90%, 91%, 92%, 93%, 94%, 95%, 96%, 97%, 98%, 99%, or 100% identity to SEQ ID NO: 456;
21) the LCDR1 comprises a sequence having at least 85%, 90%, 91%, 92%, 93%, 94%, 95%, 96%, 97%, 98%, 99%, or 100% identity to SEQ ID NO: 405, the LCDR2 comprises a sequence having at least 85%, 90%, 91%, 92%, 93%, 94%, 95%, 96%, 97%, 98%, 99%, or 100% identity to SEQ ID NO: 431, and the LCDR3 comprises a sequence having at least 85%, 90%, 91%, 92%, 93%, 94%, 95%, 96%, 97%, 98%, 99%, or 100% identity to SEQ ID NO: 457;
22) the LCDR1 comprises a sequence having at least 85%, 90%, 91%, 92%, 93%, 94%, 95%, 96%, 97%, 98%, 99%, or 100% identity to SEQ ID NO: 406, the LCDR2 comprises a sequence having at least 85%, 90%, 91%, 92%, 93%, 94%, 95%, 96%, 97%, 98%, 99%, or 100% identity to SEQ ID NO: 432, and the LCDR3 comprises a sequence having at least 85%, 90%, 91%, 92%, 93%, 94%, 95%, 96%, 97%, 98%, 99%, or 100% identity to SEQ ID NO: 458;
23) the LCDR1 comprises a sequence having at least 85%, 90%, 91%, 92%, 93%, 94%, 95%, 96%, 97%, 98%, 99%, or 100% identity to SEQ ID NO: 407, the LCDR2 comprises a sequence having at least 85%, 90%, 91%, 92%, 93%, 94%, 95%, 96%, 97%, 98%, 99%, or 100% identity to SEQ ID NO: 433, and the LCDR3 comprises a sequence having at least 85%, 90%, 91%, 92%, 93%, 94%, 95%, 96%, 97%, 98%, 99%, or 100% identity to SEQ ID NO: 459;
24) the LCDR1 comprises a sequence having at least 85%, 90%, 91%, 92%, 93%, 94%, 95%, 96%, 97%, 98%, 99%, or 100% identity to SEQ ID NO: 408, the LCDR2 comprises a sequence having at least 85%, 90%, 91%, 92%, 93%, 94%, 95%, 96%, 97%, 98%, 99%, or 100% identity to SEQ ID NO: 434, and the LCDR3 comprises a sequence having at least 85%, 90%, 91%, 92%, 93%, 94%, 95%, 96%, 97%, 98%, 99%, or 100% identity to SEQ ID NO: 460;
25) the LCDR1 comprises a sequence having at least 85%, 90%, 91%, 92%, 93%, 94%, 95%, 96%, 97%, 98%, 99%, or 100% identity to SEQ ID NO: 409, the LCDR2 comprises a sequence having at least 85%, 90%, 91%, 92%, 93%, 94%, 95%, 96%, 97%, 98%, 99%, or 100% identity to SEQ ID NO: 435, and the LCDR3 comprises a sequence having at least 85%, 90%, 91%, 92%, 93%, 94%, 95%, 96%, 97%, 98%, 99%, or 100% identity to SEQ ID NO: 461; or
26) the LCDR1 comprises a sequence having at least 85%, 90%, 91%, 92%, 93%, 94%, 95%, 96%, 97%, 98%, 99%, or 100% identity to SEQ ID NO: 410, the LCDR2 comprises a sequence having at least 85%, 90%, 91%, 92%, 93%, 94%, 95%, 96%, 97%, 98%, 99%, or 100% identity to SEQ ID NO: 436, and the LCDR3 comprises a sequence having at least 85%, 90%, 91%, 92%, 93%, 94%, 95%, 96%, 97%, 98%, 99%, or 100% identity to SEQ ID NO: 462.

In some embodiments, 1) the HCDR1 comprises a sequence having at least 85%, 90%, 91%, 92%, 93%, 94%, 95%, 96%, 97%, 98%, 99%, or 100% identity to SEQ ID NO: 104, the HCDR2 comprises a sequence having at least 85%, 90%, 91%, 92%, 93%, 94%, 95%, 96%, 97%, 98%, 99%, or 100% identity to SEQ ID NO: 130, the HCDR3 comprises a sequence having at least 85%, 90%, 91%, 92%, 93%, 94%, 95%, 96%, 97%, 98%, 99%, or 100% identity to SEQ ID NO: 156; the LCDR1 comprises a sequence having at least 85%, 90%, 91%, 92%, 93%, 94%, 95%, 96%, 97%, 98%, 99%, or 100% identity to SEQ ID NO: 385, the LCDR2 comprises a sequence having at least 85%, 90%, 91%, 92%, 93%, 94%, 95%, 96%, 97%, 98%, 99%, or 100% identity to SEQ ID NO: 411, and the LCDR3 comprises a sequence having at least 85%, 90%, 91%, 92%, 93%, 94%, 95%, 96%, 97%, 98%, 99%, or 100% identity to SEQ ID NO: 437;
2) the HCDR1 comprises a sequence having at least 85%, 90%, 91%, 92%, 93%, 94%, 95%, 96%, 97%, 98%, 99%, or 100% identity to SEQ ID NO: 105, the HCDR2 comprises a sequence having at least 85%, 90%, 91%, 92%, 93%, 94%, 95%, 96%, 97%, 98%, 99%, or 100% identity to SEQ ID NO: 131, the HCDR3 comprises a sequence having at least 85%, 90%, 91%, 92%, 93%, 94%, 95%, 96%, 97%, 98%, 99%, or 100% identity to SEQ ID NO: 157; the LCDR1 comprises a sequence having at least 85%, 90%, 91%, 92%, 93%, 94%, 95%, 96%, 97%, 98%, 99%, or 100% identity to SEQ ID NO: 386, the LCDR2 comprises a sequence having at least 85%, 90%, 91%, 92%, 93%, 94%, 95%, 96%, 97%, 98%, 99%, or 100% identity to SEQ ID NO: 412, and the LCDR3 comprises a sequence having at least 85%, 90%, 91%, 92%, 93%, 94%, 95%, 96%, 97%, 98%, 99%, or 100% identity to SEQ ID NO: 438;
3) the HCDR1 comprises a sequence having at least 85%, 90%, 91%, 92%, 93%, 94%, 95%, 96%, 97%, 98%, 99%, or 100% identity to SEQ ID NO: 106, the HCDR2 comprises a sequence having at least 85%, 90%, 91%, 92%, 93%, 94%, 95%, 96%, 97%, 98%, 99%, or 100% identity to SEQ ID NO: 132, the HCDR3 comprises a sequence having at least 85%, 90%, 91%, 92%, 93%, 94%, 95%, 96%, 97%, 98%, 99%, or 100% identity to SEQ ID NO: 158; the LCDR1 comprises a sequence having at least 85%, 90%, 91%, 92%, 93%, 94%, 95%, 96%, 97%, 98%, 99%, or 100% identity to SEQ ID NO: 387, the LCDR2 comprises a sequence having at least 85%, 90%, 91%, 92%, 93%, 94%, 95%, 96%, 97%, 98%, 99%, or 100% identity to SEQ ID NO: 413, and the LCDR3 comprises a sequence having at least 85%, 90%, 91%, 92%, 93%, 94%, 95%, 96%, 97%, 98%, 99%, or 100% identity to SEQ ID NO: 439;
4) the HCDR1 comprises a sequence having at least 85%, 90%, 91%, 92%, 93%, 94%, 95%, 96%, 97%, 98%, 99%, or 100% identity to SEQ ID NO: 107, the HCDR2 comprises a sequence having at least 85%, 90%, 91%, 92%, 93%, 94%, 95%, 96%, 97%, 98%, 99%, or 100% identity to SEQ ID NO: 133, the HCDR3 comprises a sequence having at least 85%, 90%, 91%, 92%, 93%, 94%, 95%, 96%, 97%, 98%, 99%, or 100% identity to SEQ ID NO: 159; the LCDR1 comprises a sequence having at least 85%, 90%, 91%, 92%, 93%, 94%, 95%, 96%, 97%, 98%, 99%, or 100% identity to SEQ ID NO: 388, the LCDR2 comprises a sequence having at least 85%, 90%, 91%, 92%, 93%, 94%, 95%, 96%, 97%, 98%, 99%, or 100% identity to SEQ ID NO: 414, and the LCDR3 comprises a sequence having at least 85%, 90%, 91%, 92%, 93%, 94%, 95%, 96%, 97%, 98%, 99%, or 100% identity to SEQ ID NO: 440;
5) the HCDR1 comprises a sequence having at least 85%, 90%, 91%, 92%, 93%, 94%, 95%, 96%, 97%, 98%, 99%, or 100% identity to SEQ ID NO: 108, the HCDR2 comprises a sequence having at least 85%, 90%, 91%, 92%, 93%, 94%, 95%, 96%, 97%, 98%, 99%, or 100% identity to SEQ ID NO: 134, the HCDR3 comprises a sequence having at least 85%, 90%, 91%, 92%, 93%, 94%, 95%, 96%, 97%, 98%, 99%, or 100% identity to SEQ ID NO: 160; the LCDR1 comprises a sequence having at least 85%, 90%, 91%, 92%, 93%, 94%, 95%, 96%, 97%, 98%, 99%, or 100% identity to SEQ ID NO: 389, the LCDR2 comprises a sequence having at least 85%, 90%, 91%, 92%, 93%, 94%, 95%, 96%, 97%, 98%, 99%, or 100% identity to SEQ ID NO: 415, and the LCDR3 comprises a sequence having at least 85%, 90%, 91%, 92%, 93%, 94%, 95%, 96%, 97%, 98%, 99%, or 100% identity to SEQ ID NO: 441;
6) the HCDR1 comprises a sequence having at least 85%, 90%, 91%, 92%, 93%, 94%, 95%, 96%, 97%, 98%, 99%, or 100% identity to SEQ ID NO: 109, the HCDR2 comprises a sequence having at least 85%, 90%, 91%, 92%, 93%, 94%, 95%, 96%, 97%, 98%, 99%, or 100% identity to SEQ ID NO: 135, the HCDR3 comprises a sequence having at least 85%, 90%, 91%, 92%, 93%, 94%, 95%, 96%, 97%, 98%, 99%, or 100% identity to SEQ ID NO: 161; the LCDR1 comprises a sequence having at least 85%, 90%, 91%, 92%, 93%, 94%, 95%, 96%, 97%, 98%, 99%, or 100% identity to SEQ ID NO: 390, the LCDR2 comprises a sequence having at least 85%, 90%, 91%, 92%, 93%, 94%, 95%, 96%, 97%, 98%, 99%, or 100% identity to SEQ ID NO: 416, and the LCDR3 comprises a sequence having at least 85%, 90%, 91%, 92%, 93%, 94%, 95%, 96%, 97%, 98%, 99%, or 100% identity to SEQ ID NO: 442;
7) the HCDR1 comprises a sequence having at least 85%, 90%, 91%, 92%, 93%, 94%, 95%, 96%, 97%, 98%, 99%, or 100% identity to SEQ ID NO: 110, the HCDR2 comprises a sequence having at least 85%, 90%, 91%, 92%, 93%, 94%, 95%, 96%, 97%, 98%, 99%, or 100% identity to SEQ ID NO: 136, the HCDR3 comprises a sequence having at least 85%, 90%, 91%, 92%, 93%, 94%, 95%, 96%, 97%, 98%, 99%, or 100% identity to SEQ ID NO: 162; the LCDR1 comprises a sequence having at least 85%, 90%, 91%, 92%, 93%, 94%, 95%, 96%, 97%, 98%, 99%, or 100% identity to SEQ ID NO: 391, the LCDR2 comprises a sequence having at least 85%, 90%, 91%, 92%, 93%, 94%, 95%, 96%, 97%, 98%, 99%, or 100% identity to SEQ ID NO: 417, and the LCDR3 comprises a sequence having at least 85%, 90%, 91%, 92%, 93%, 94%, 95%, 96%, 97%, 98%, 99%, or 100% identity to SEQ ID NO: 443;
8) the HCDR1 comprises a sequence having at least 85%, 90%, 91%, 92%, 93%, 94%, 95%, 96%, 97%, 98%, 99%, or 100% identity to SEQ ID NO: 111, the HCDR2 comprises a sequence having at least 85%, 90%, 91%, 92%, 93%, 94%, 95%, 96%, 97%, 98%, 99%, or 100% identity to SEQ ID NO: 137, the HCDR3 comprises a sequence having at least 85%, 90%, 91%, 92%, 93%, 94%, 95%, 96%, 97%, 98%, 99%, or 100% identity to SEQ ID NO: 163; the LCDR1 comprises a sequence having at least 85%, 90%, 91%, 92%, 93%, 94%, 95%, 96%, 97%, 98%, 99%, or 100% identity to SEQ ID NO: 392, the LCDR2 comprises a sequence having at least 85%, 90%, 91%, 92%, 93%, 94%, 95%, 96%, 97%, 98%, 99%, or 100% identity to SEQ ID NO: 418, and the LCDR3 comprises a sequence having at least 85%, 90%, 91%, 92%, 93%, 94%, 95%, 96%, 97%, 98%, 99%, or 100% identity to SEQ ID NO: 444;
9) the HCDR1 comprises a sequence having at least 85%, 90%, 91%, 92%, 93%, 94%, 95%, 96%, 97%, 98%, 99%, or 100% identity to SEQ ID NO: 112, the HCDR2 comprises a sequence having at least 85%, 90%, 91%, 92%, 93%, 94%, 95%, 96%, 97%, 98%, 99%, or 100% identity to SEQ ID NO: 138, the HCDR3 comprises a sequence having at least 85%, 90%, 91%, 92%, 93%, 94%, 95%, 96%, 97%, 98%, 99%, or 100% identity to SEQ ID NO: 164; the LCDR1 comprises a sequence having at least 85%, 90%, 91%, 92%, 93%, 94%, 95%, 96%, 97%, 98%, 99%, or 100% identity to SEQ ID NO: 393, the LCDR2 comprises a sequence having at least 85%, 90%, 91%, 92%, 93%, 94%, 95%, 96%, 97%, 98%, 99%, or 100% identity to SEQ ID NO: 419, and the LCDR3 comprises a sequence having at least 85%, 90%, 91%, 92%, 93%, 94%, 95%, 96%, 97%, 98%, 99%, or 100% identity to SEQ ID NO: 445;
10) the HCDR1 comprises a sequence having at least 85%, 90%, 91%, 92%, 93%, 94%, 95%, 96%, 97%, 98%, 99%, or 100% identity to SEQ ID NO: 113, the HCDR2 comprises a sequence having at least 85%, 90%, 91%, 92%, 93%, 94%, 95%, 96%, 97%, 98%, 99%, or 100% identity to SEQ ID NO: 139, the HCDR3 comprises a sequence having at least 85%, 90%, 91%, 92%, 93%, 94%, 95%, 96%, 97%, 98%, 99%, or 100% identity to SEQ ID NO: 165; the LCDR1 comprises a sequence having at least 85%, 90%, 91%, 92%, 93%, 94%, 95%, 96%, 97%, 98%, 99%, or 100% identity to SEQ ID NO: 394, the LCDR2 comprises a sequence having at least 85%, 90%, 91%, 92%, 93%, 94%, 95%, 96%, 97%, 98%, 99%, or 100% identity to SEQ ID NO: 420, and the LCDR3 comprises a sequence having at least 85%, 90%, 91%, 92%, 93%, 94%, 95%, 96%, 97%, 98%, 99%, or 100% identity to SEQ ID NO: 446;
11) the HCDR1 comprises a sequence having at least 85%, 90%, 91%, 92%, 93%, 94%, 95%, 96%, 97%, 98%, 99%, or 100% identity to SEQ ID NO: 114, the HCDR2 comprises a sequence having at least 85%, 90%, 91%, 92%, 93%, 94%, 95%, 96%, 97%, 98%, 99%, or 100% identity to SEQ ID NO: 140, the HCDR3 comprises a sequence having at least 85%, 90%, 91%, 92%, 93%, 94%, 95%, 96%, 97%, 98%, 99%, or 100% identity to SEQ ID NO: 166; the LCDR1 comprises a sequence having at least 85%, 90%, 91%, 92%, 93%, 94%, 95%, 96%, 97%, 98%, 99%, or 100% identity to SEQ ID NO: 395, the LCDR2 comprises a sequence having at least 85%, 90%, 91%, 92%, 93%, 94%, 95%, 96%, 97%, 98%, 99%, or 100% identity to SEQ ID NO: 421, and the LCDR3 comprises a sequence having at least 85%, 90%, 91%, 92%, 93%, 94%, 95%, 96%, 97%, 98%, 99%, or 100% identity to SEQ ID NO: 447;
12) the HCDR1 comprises a sequence having at least 85%, 90%, 91%, 92%, 93%, 94%, 95%, 96%, 97%, 98%, 99%, or 100% identity to SEQ ID NO: 115, the HCDR2 comprises a sequence having at least 85%, 90%, 91%, 92%, 93%, 94%, 95%, 96%, 97%, 98%, 99%, or 100% identity to SEQ ID NO: 141, the HCDR3 comprises a sequence having at least 85%, 90%, 91%, 92%, 93%, 94%, 95%, 96%, 97%, 98%, 99%, or 100% identity to SEQ ID NO: 167; the LCDR1 comprises a sequence having at least 85%, 90%, 91%, 92%, 93%, 94%, 95%, 96%, 97%, 98%, 99%, or 100% identity to SEQ ID NO: 396, the LCDR2 comprises a sequence having at least 85%, 90%, 91%, 92%, 93%, 94%, 95%, 96%, 97%, 98%, 99%, or 100% identity to SEQ ID NO: 422, and the LCDR3 comprises a sequence having at least 85%, 90%, 91%, 92%, 93%, 94%, 95%, 96%, 97%, 98%, 99%, or 100% identity to SEQ ID NO: 448;
13) the HCDR1 comprises a sequence having at least 85%, 90%, 91%, 92%, 93%, 94%, 95%, 96%, 97%, 98%, 99%, or 100% identity to SEQ ID NO: 116, the HCDR2 comprises a sequence having at least 85%, 90%, 91%, 92%, 93%, 94%, 95%, 96%, 97%, 98%, 99%, or 100% identity to SEQ ID NO: 142, the HCDR3 comprises a sequence having at least 85%, 90%, 91%, 92%, 93%, 94%, 95%, 96%, 97%, 98%, 99%, or 100% identity to SEQ ID NO: 168; the LCDR1 comprises a sequence having at least 85%, 90%, 91%, 92%, 93%, 94%, 95%, 96%, 97%, 98%, 99%, or 100% identity to SEQ ID NO: 397, the LCDR2 comprises a sequence having at least 85%, 90%, 91%, 92%, 93%, 94%, 95%, 96%, 97%, 98%, 99%, or 100% identity to SEQ ID NO: 423, and the LCDR3 comprises a sequence having at least 85%, 90%, 91%, 92%, 93%, 94%, 95%, 96%, 97%, 98%, 99%, or 100% identity to SEQ ID NO: 449;
14) the HCDR1 comprises a sequence having at least 85%, 90%, 91%, 92%, 93%, 94%, 95%, 96%, 97%, 98%, 99%, or 100% identity to SEQ ID NO: 117, the HCDR2 comprises a sequence having at least 85%, 90%, 91%, 92%, 93%, 94%, 95%, 96%, 97%, 98%, 99%, or 100% identity to SEQ ID NO: 143, the HCDR3 comprises a sequence having at least 85%, 90%, 91%, 92%, 93%, 94%, 95%, 96%, 97%, 98%, 99%, or 100% identity to SEQ ID NO: 169; the LCDR1 comprises a sequence having at least 85%, 90%, 91%, 92%, 93%, 94%, 95%, 96%, 97%, 98%, 99%, or 100% identity to SEQ ID NO: 398, the LCDR2 comprises a sequence having at least 85%, 90%, 91%, 92%, 93%, 94%, 95%, 96%, 97%, 98%, 99%, or 100% identity to SEQ ID NO: 424, and the LCDR3 comprises a sequence having at least 85%, 90%, 91%, 92%, 93%, 94%, 95%, 96%, 97%, 98%, 99%, or 100% identity to SEQ ID NO: 450;
15) the HCDR1 comprises a sequence having at least 85%, 90%, 91%, 92%, 93%, 94%, 95%, 96%, 97%, 98%, 99%, or 100% identity to SEQ ID NO: 118, the HCDR2 comprises a sequence having at least 85%, 90%, 91%, 92%, 93%, 94%, 95%, 96%, 97%, 98%, 99%, or 100% identity to SEQ ID NO: 144, the HCDR3 comprises a sequence having at least 85%, 90%, 91%, 92%, 93%, 94%, 95%, 96%, 97%, 98%, 99%, or 100% identity to SEQ ID NO: 170; the LCDR1 comprises a sequence having at least 85%, 90%, 91%, 92%, 93%, 94%, 95%, 96%, 97%, 98%, 99%, or 100% identity to SEQ ID NO: 399, the LCDR2 comprises a sequence having at least 85%, 90%, 91%, 92%, 93%, 94%, 95%, 96%, 97%, 98%, 99%, or 100% identity to SEQ ID NO: 425, and the LCDR3 comprises a sequence having at least 85%, 90%, 91%, 92%, 93%, 94%, 95%, 96%, 97%, 98%, 99%, or 100% identity to SEQ ID NO: 451;
16) the HCDR1 comprises a sequence having at least 85%, 90%, 91%, 92%, 93%, 94%, 95%, 96%, 97%, 98%, 99%, or 100% identity to SEQ ID NO: 119, the HCDR2 comprises a sequence having at least 85%, 90%, 91%, 92%, 93%, 94%, 95%, 96%, 97%, 98%, 99%, or 100% identity to SEQ ID NO: 145, the HCDR3 comprises a sequence having at least 85%, 90%, 91%, 92%, 93%, 94%, 95%, 96%, 97%, 98%, 99%, or 100% identity to SEQ ID NO: 171; the LCDR1 comprises a sequence having at least 85%, 90%, 91%, 92%, 93%, 94%, 95%, 96%, 97%, 98%, 99%, or 100% identity to SEQ ID NO: 400, the LCDR2 comprises a sequence having at least 85%, 90%, 91%, 92%, 93%, 94%, 95%, 96%, 97%, 98%, 99%, or 100% identity to SEQ ID NO: 426, and the LCDR3 comprises a sequence having at least 85%, 90%, 91%, 92%, 93%, 94%, 95%, 96%, 97%, 98%, 99%, or 100% identity to SEQ ID NO: 452;
17) the HCDR1 comprises a sequence having at least 85%, 90%, 91%, 92%, 93%, 94%, 95%, 96%, 97%, 98%, 99%, or 100% identity to SEQ ID NO: 120, the HCDR2 comprises a sequence having at least 85%, 90%, 91%, 92%, 93%, 94%, 95%, 96%, 97%, 98%, 99%, or 100% identity to SEQ ID NO: 146, the HCDR3 comprises a sequence having at least 85%, 90%, 91%, 92%, 93%, 94%, 95%, 96%, 97%, 98%, 99%, or 100% identity to SEQ ID NO: 172; the LCDR1 comprises a sequence having at least 85%, 90%, 91%, 92%, 93%, 94%, 95%, 96%, 97%, 98%, 99%, or 100% identity to SEQ ID NO: 401, the LCDR2 comprises a sequence having at least 85%, 90%, 91%, 92%, 93%, 94%, 95%, 96%, 97%, 98%, 99%, or 100% identity to SEQ ID NO: 427, and the LCDR3 comprises a sequence having at least 85%, 90%, 91%, 92%, 93%, 94%, 95%, 96%, 97%, 98%, 99%, or 100% identity to SEQ ID NO: 453;
18) the HCDR1 comprises a sequence having at least 85%, 90%, 91%, 92%, 93%, 94%, 95%, 96%, 97%, 98%, 99%, or 100% identity to SEQ ID NO: 121, the HCDR2 comprises a sequence having at least 85%, 90%, 91%, 92%, 93%, 94%, 95%, 96%, 97%, 98%, 99%, or 100% identity to SEQ ID NO: 147, the HCDR3 comprises a sequence having at least 85%, 90%, 91%, 92%, 93%, 94%, 95%, 96%, 97%, 98%, 99%, or 100% identity to SEQ ID NO: 173; the LCDR1 comprises a sequence having at least 85%, 90%, 91%, 92%, 93%, 94%, 95%, 96%, 97%, 98%, 99%, or 100% identity to SEQ ID NO: 402, the LCDR2 comprises a sequence having at least 85%, 90%, 91%, 92%, 93%, 94%, 95%, 96%, 97%, 98%, 99%, or 100% identity to SEQ ID NO: 428, and the LCDR3 comprises a sequence having at least 85%, 90%, 91%, 92%, 93%, 94%, 95%, 96%, 97%, 98%, 99%, or 100% identity to SEQ ID NO: 454;
19) the HCDR1 comprises a sequence having at least 85%, 90%, 91%, 92%, 93%, 94%, 95%, 96%, 97%, 98%, 99%, or 100% identity to SEQ ID NO: 122, the HCDR2 comprises a sequence having at least 85%, 90%, 91%, 92%, 93%, 94%, 95%, 96%, 97%, 98%, 99%, or 100% identity to SEQ ID NO: 148, the HCDR3 comprises a sequence having at least 85%, 90%, 91%, 92%, 93%, 94%, 95%, 96%, 97%, 98%, 99%, or 100% identity to SEQ ID NO: 174; the LCDR1 comprises a sequence having at least 85%, 90%, 91%, 92%, 93%, 94%, 95%, 96%, 97%, 98%, 99%, or 100% identity to SEQ ID NO: 403, the LCDR2 comprises a sequence having at least 85%, 90%, 91%, 92%, 93%, 94%, 95%, 96%, 97%, 98%, 99%, or 100% identity to SEQ ID NO: 429, and the LCDR3 comprises a sequence having at least 85%, 90%, 91%, 92%, 93%, 94%, 95%, 96%, 97%, 98%, 99%, or 100% identity to SEQ ID NO: 455;
20) the HCDR1 comprises a sequence having at least 85%, 90%, 91%, 92%, 93%, 94%, 95%, 96%, 97%, 98%, 99%, or 100% identity to SEQ ID NO: 123, the HCDR2 comprises a sequence having at least 85%, 90%, 91%, 92%, 93%, 94%, 95%, 96%, 97%, 98%, 99%, or 100% identity to SEQ ID NO: 149, the HCDR3 comprises a sequence having at least 85%, 90%, 91%, 92%, 93%, 94%, 95%, 96%, 97%, 98%, 99%, or 100% identity to SEQ ID NO: 175; the LCDR1 comprises a sequence having at least 85%, 90%, 91%, 92%, 93%, 94%, 95%, 96%, 97%, 98%, 99%, or 100% identity to SEQ ID NO: 404, the LCDR2 comprises a sequence having at least 85%, 90%, 91%, 92%, 93%, 94%, 95%, 96%, 97%, 98%, 99%, or 100% identity to SEQ ID NO: 430, and the LCDR3 comprises a sequence having at least 85%, 90%, 91%, 92%, 93%, 94%, 95%, 96%, 97%, 98%, 99%, or 100% identity to SEQ ID NO: 456;
21) the HCDR1 comprises a sequence having at least 85%, 90%, 91%, 92%, 93%, 94%, 95%, 96%, 97%, 98%, 99%, or 100% identity to SEQ ID NO: 124, the HCDR2 comprises a sequence having at least 85%, 90%, 91%, 92%, 93%, 94%, 95%, 96%, 97%, 98%, 99%, or 100% identity to SEQ ID NO: 150, the HCDR3 comprises a sequence having at least 85%, 90%, 91%, 92%, 93%, 94%, 95%, 96%, 97%, 98%, 99%, or 100% identity to SEQ ID NO: 176; the LCDR1 comprises a sequence having at least 85%, 90%, 91%, 92%, 93%, 94%, 95%, 96%, 97%, 98%, 99%, or 100% identity to SEQ ID NO: 405, the LCDR2 comprises a sequence having at least 85%, 90%, 91%, 92%, 93%, 94%, 95%, 96%, 97%, 98%, 99%, or 100% identity to SEQ ID NO: 431, and the LCDR3 comprises a sequence having at least 85%, 90%, 91%, 92%, 93%, 94%, 95%, 96%, 97%, 98%, 99%, or 100% identity to SEQ ID NO: 457;
22) the HCDR1 comprises a sequence having at least 85%, 90%, 91%, 92%, 93%, 94%, 95%, 96%, 97%, 98%, 99%, or 100% identity to SEQ ID NO: 125, the HCDR2 comprises a sequence having at least 85%, 90%, 91%, 92%, 93%, 94%, 95%, 96%, 97%, 98%, 99%, or 100% identity to SEQ ID NO: 151, the HCDR3 comprises a sequence having at least 85%, 90%, 91%, 92%, 93%, 94%, 95%, 96%, 97%, 98%, 99%, or 100% identity to SEQ ID NO: 177; the LCDR1 comprises a sequence having at least 85%, 90%, 91%, 92%, 93%, 94%, 95%, 96%, 97%, 98%, 99%, or 100% identity to SEQ ID NO: 406, the LCDR2 comprises a sequence having at least 85%, 90%, 91%, 92%, 93%, 94%, 95%, 96%, 97%, 98%, 99%, or 100% identity to SEQ ID NO: 432, and the LCDR3 comprises a sequence having at least 85%, 90%, 91%, 92%, 93%, 94%, 95%, 96%, 97%, 98%, 99%, or 100% identity to SEQ ID NO: 458;
23) the HCDR1 comprises a sequence having at least 85%, 90%, 91%, 92%, 93%, 94%, 95%, 96%, 97%, 98%, 99%, or 100% identity to SEQ ID NO: 126, the HCDR2 comprises a sequence having at least 85%, 90%, 91%, 92%, 93%, 94%, 95%, 96%, 97%, 98%, 99%, or 100% identity to SEQ ID NO: 152, the HCDR3 comprises a sequence having at least 85%, 90%, 91%, 92%, 93%, 94%, 95%, 96%, 97%, 98%, 99%, or 100% identity to SEQ ID NO: 178; the LCDR1 comprises a sequence having at least 85%, 90%, 91%, 92%, 93%, 94%, 95%, 96%, 97%, 98%, 99%, or 100% identity to SEQ ID NO: 407, the LCDR2 comprises a sequence having at least 85%, 90%, 91%, 92%, 93%, 94%, 95%, 96%, 97%, 98%, 99%, or 100% identity to SEQ ID NO: 433, and the LCDR3 comprises a sequence having at least 85%, 90%, 91%, 92%, 93%, 94%, 95%, 96%, 97%, 98%, 99%, or 100% identity to SEQ ID NO: 459;
24) the HCDR1 comprises a sequence having at least 85%, 90%, 91%, 92%, 93%, 94%, 95%, 96%, 97%, 98%, 99%, or 100% identity to SEQ ID NO: 127, the HCDR2 comprises a sequence having at least 85%, 90%, 91%, 92%, 93%, 94%, 95%, 96%, 97%, 98%, 99%, or 100% identity to SEQ ID NO: 153, the HCDR3 comprises a sequence having at least 85%, 90%, 91%, 92%, 93%, 94%, 95%, 96%, 97%, 98%, 99%, or 100% identity to SEQ ID NO: 179; the LCDR1 comprises a sequence having at least 85%, 90%, 91%, 92%, 93%, 94%, 95%, 96%, 97%, 98%, 99%, or 100% identity to SEQ ID NO: 408, the LCDR2 comprises a sequence having at least 85%, 90%, 91%, 92%, 93%, 94%, 95%, 96%, 97%, 98%, 99%, or 100% identity to SEQ ID NO: 434, and the LCDR3 comprises a sequence having at least 85%, 90%, 91%, 92%, 93%, 94%, 95%, 96%, 97%, 98%, 99%, or 100% identity to SEQ ID NO: 460;
25) the HCDR1 comprises a sequence having at least 85%, 90%, 91%, 92%, 93%, 94%, 95%, 96%, 97%, 98%, 99%, or 100% identity to SEQ ID NO: 128, the HCDR2 comprises a sequence having at least 85%, 90%, 91%, 92%, 93%, 94%, 95%, 96%, 97%, 98%, 99%, or 100% identity to SEQ ID NO: 154, the HCDR3 comprises a sequence having at least 85%, 90%, 91%, 92%, 93%, 94%, 95%, 96%, 97%, 98%, 99%, or 100% identity to SEQ ID NO: 180; the LCDR1 comprises a sequence having at least 85%, 90%, 91%, 92%, 93%, 94%, 95%, 96%, 97%, 98%, 99%, or 100% identity to SEQ ID NO: 409, the LCDR2 comprises a sequence having at least 85%, 90%, 91%, 92%, 93%, 94%, 95%, 96%, 97%, 98%, 99%, or 100% identity to SEQ ID NO: 435, and the LCDR3 comprises a sequence having at least 85%, 90%, 91%, 92%, 93%, 94%, 95%, 96%, 97%, 98%, 99%, or 100% identity to SEQ ID NO: 461; or
26) the HCDR1 comprises a sequence having at least 85%, 90%, 91%, 92%, 93%, 94%, 95%, 96%, 97%, 98%, 99%, or 100% identity to SEQ ID NO: 129, the HCDR2 comprises a sequence having at least 85%, 90%, 91%, 92%, 93%, 94%, 95%, 96%, 97%, 98%, 99%, or 100% identity to SEQ ID NO: 155, the HCDR3 comprises a sequence having at least 85%, 90%, 91%, 92%, 93%, 94%, 95%, 96%, 97%, 98%, 99%, or 100% identity to SEQ ID NO: 181, the LCDR1 comprises a sequence having at least 85%, 90%, 91%, 92%, 93%, 94%, 95%, 96%, 97%, 98%, 99%, or 100% identity to SEQ ID NO: 410, the LCDR2 comprises a sequence having at least 85%, 90%, 91%, 92%, 93%, 94%, 95%, 96%, 97%, 98%, 99%, or 100% identity to SEQ ID NO: 436, and the LCDR3 comprises a sequence having at least 85%, 90%, 91%, 92%, 93%, 94%, 95%, 96%, 97%, 98%, 99%, or 100% identity to SEQ ID NO: 462.

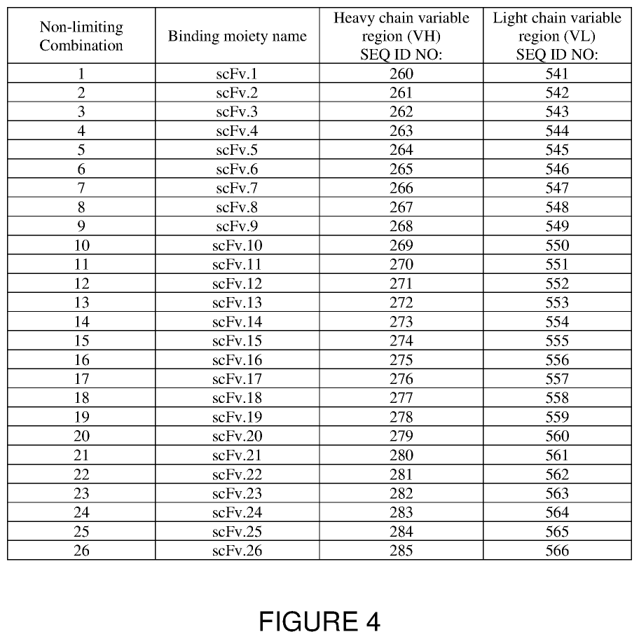

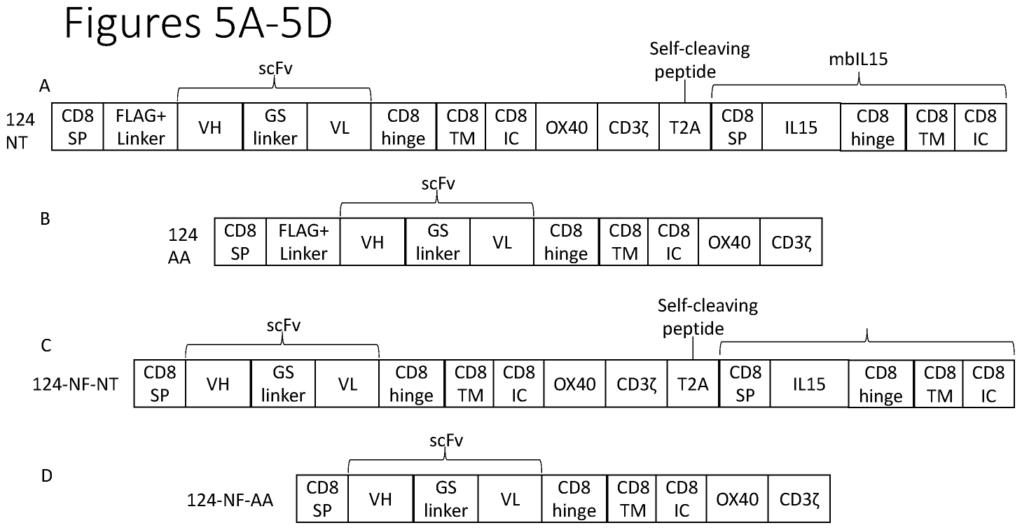

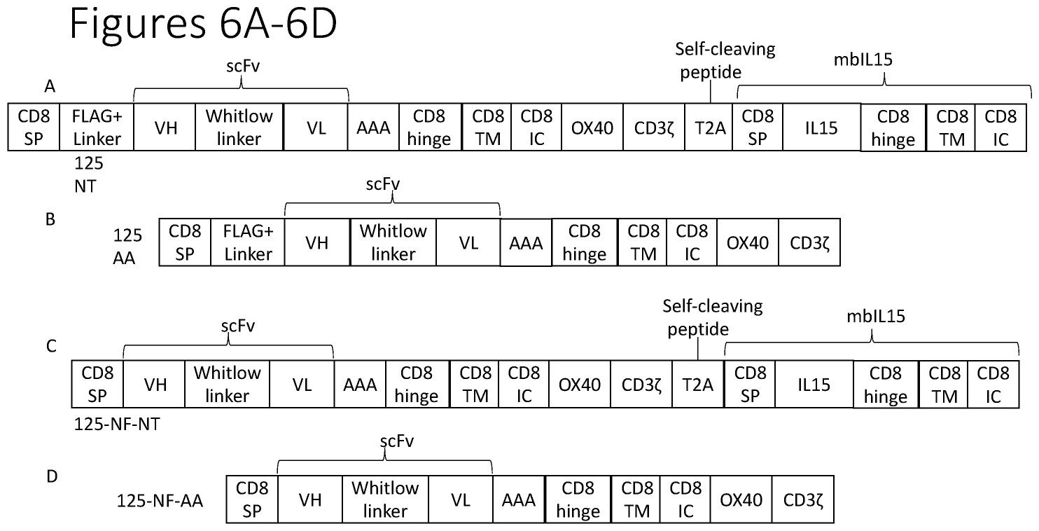

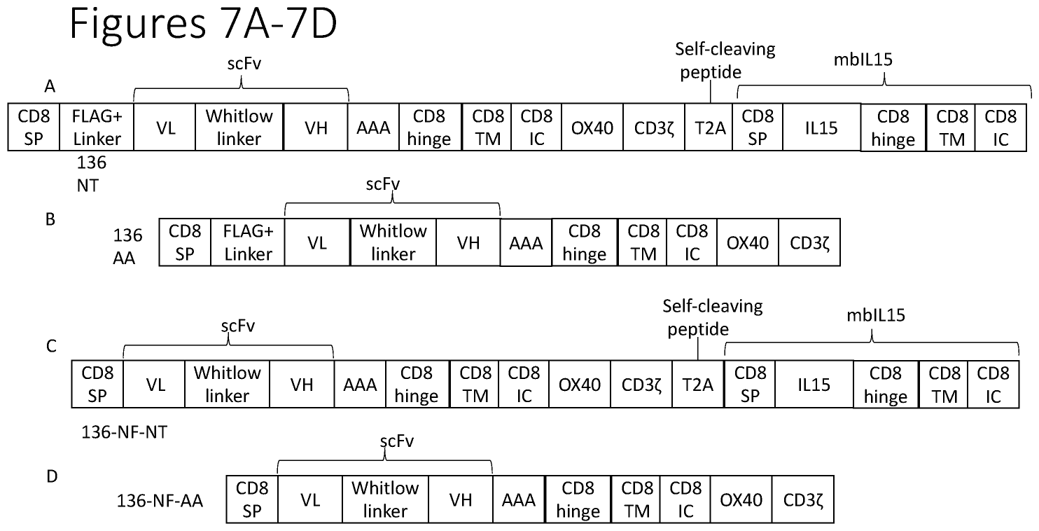

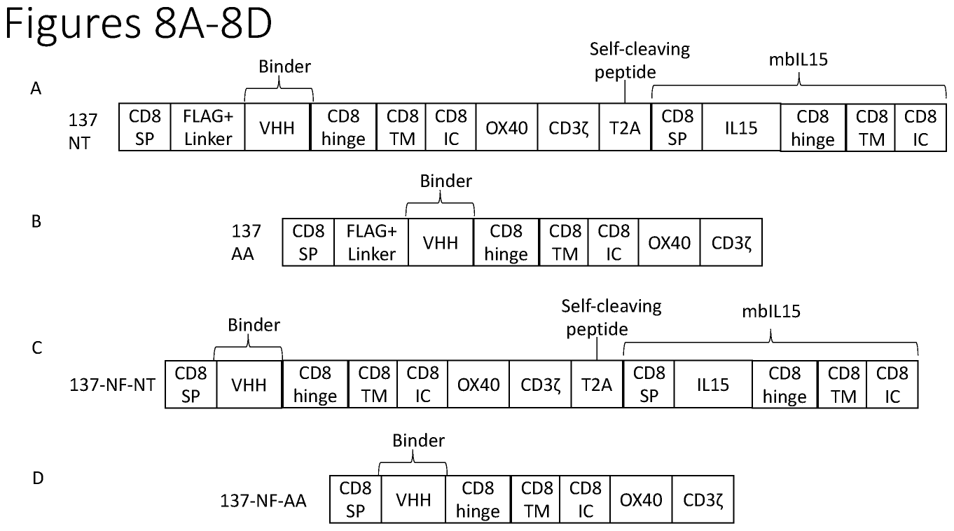

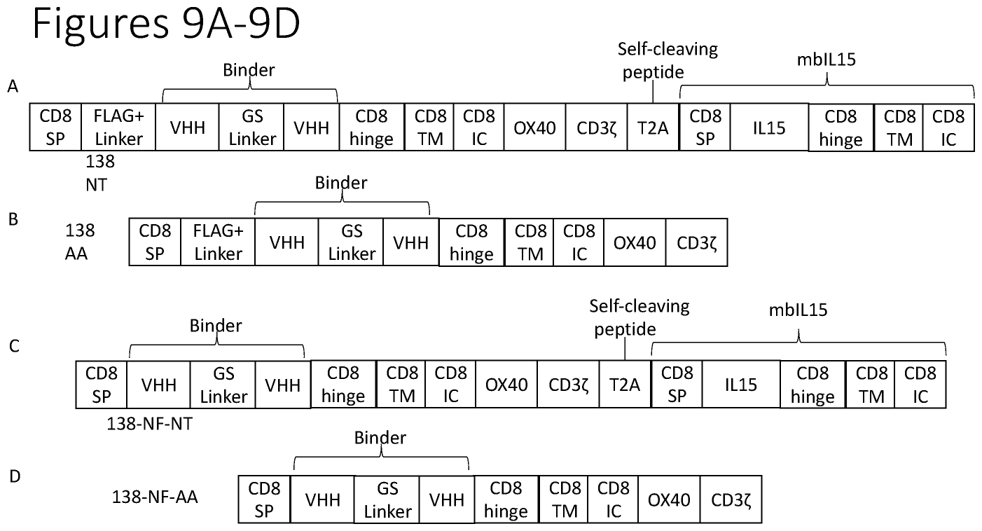

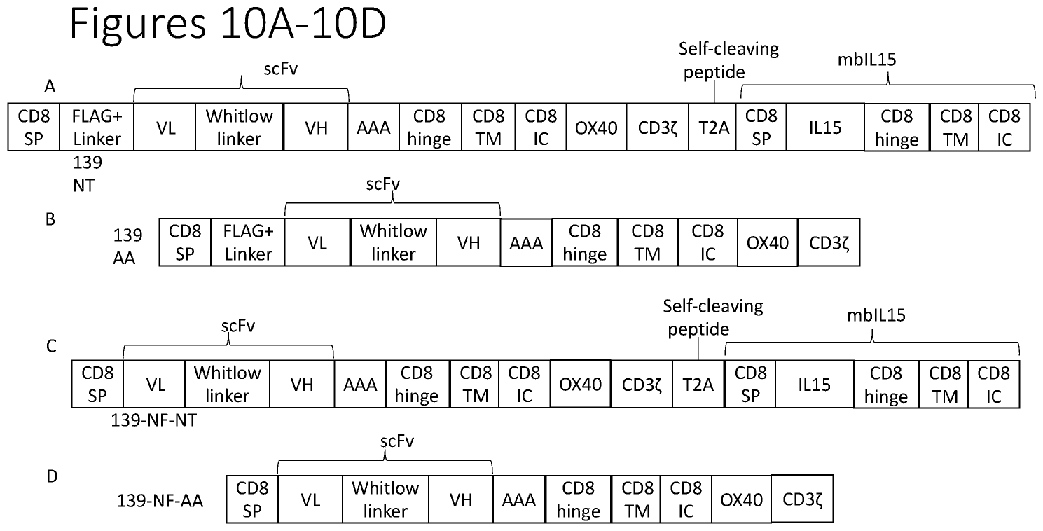

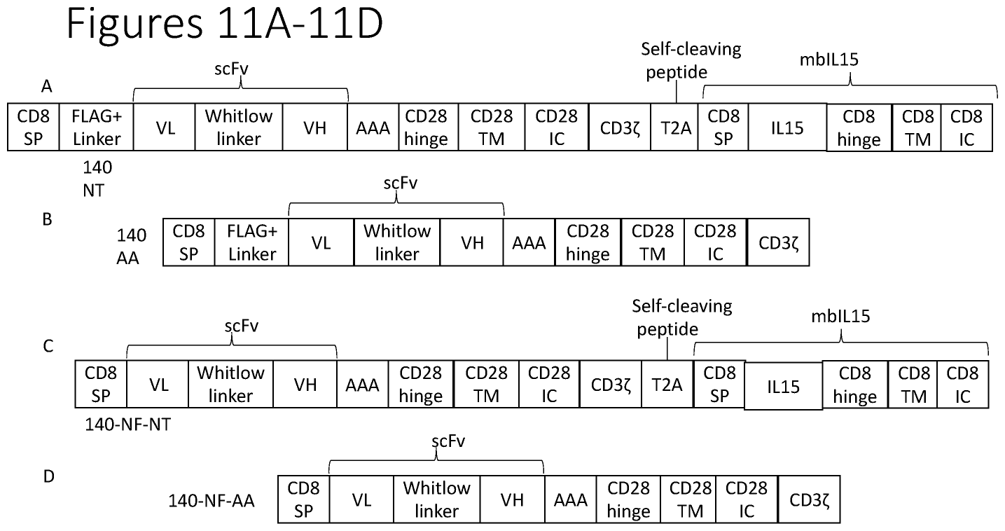

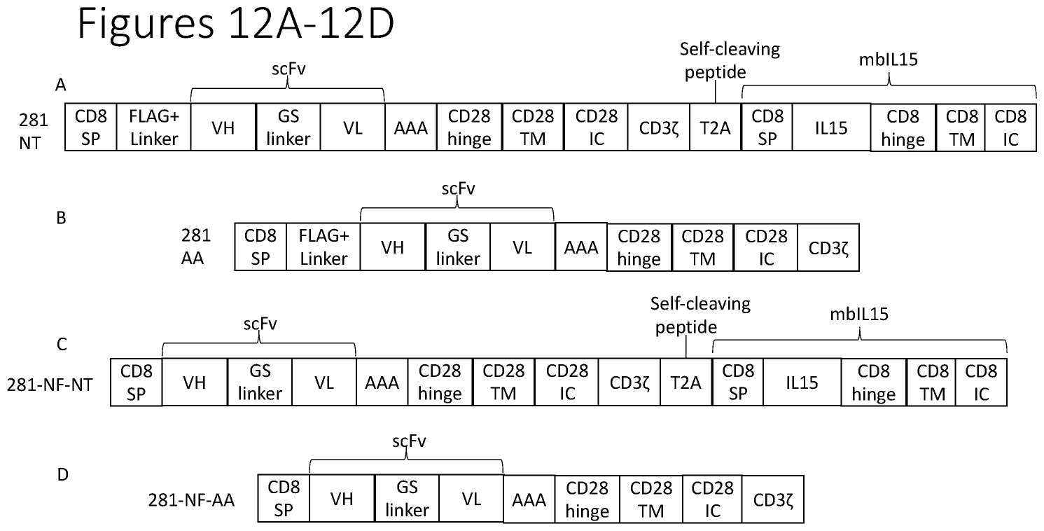

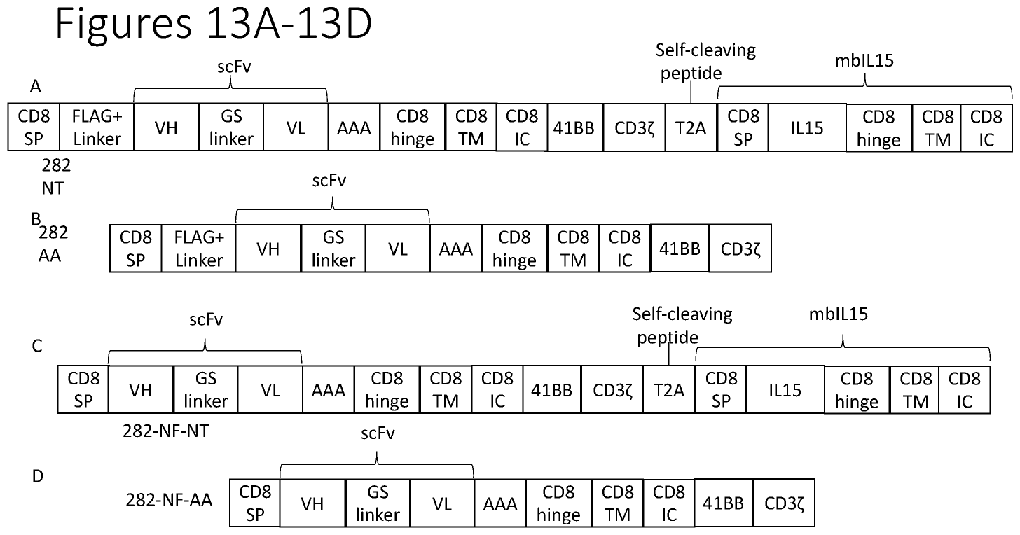

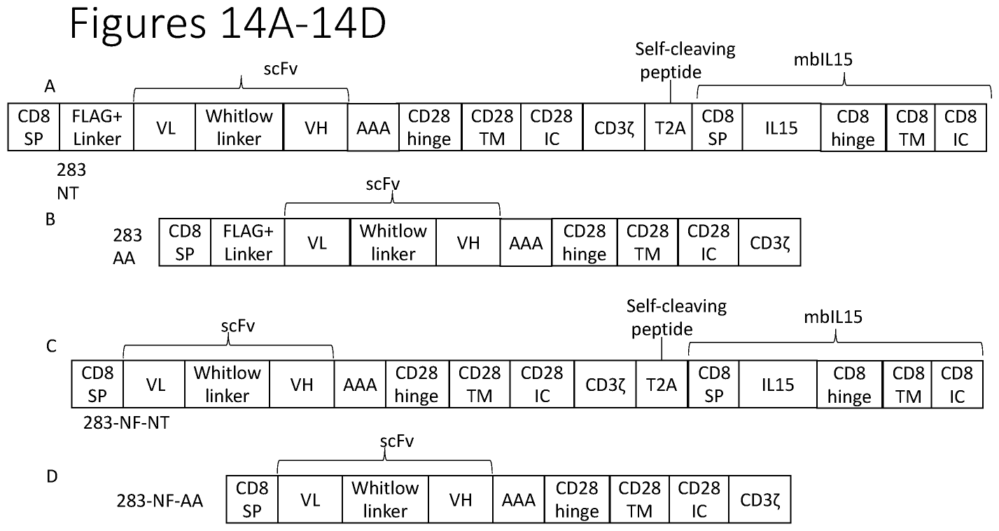

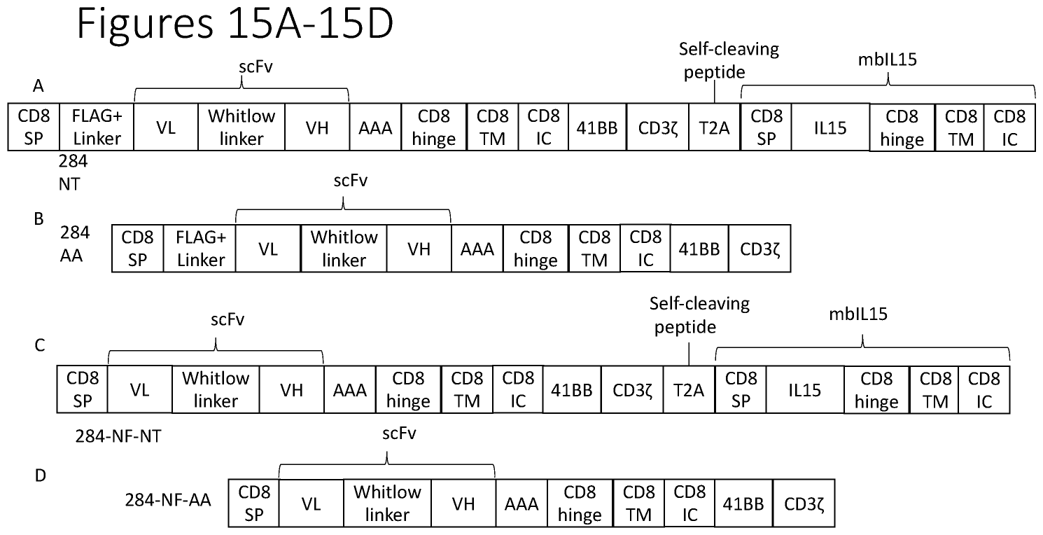

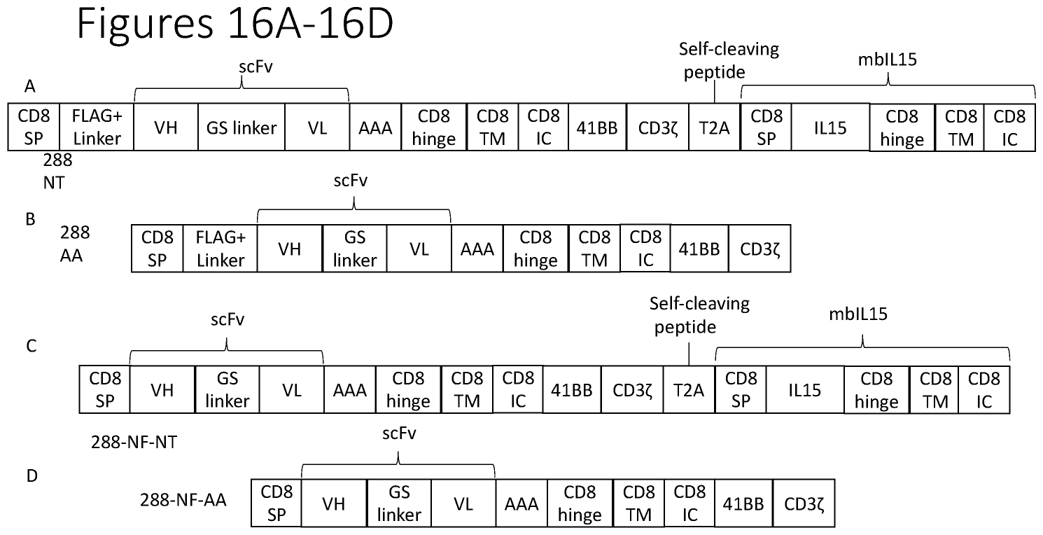

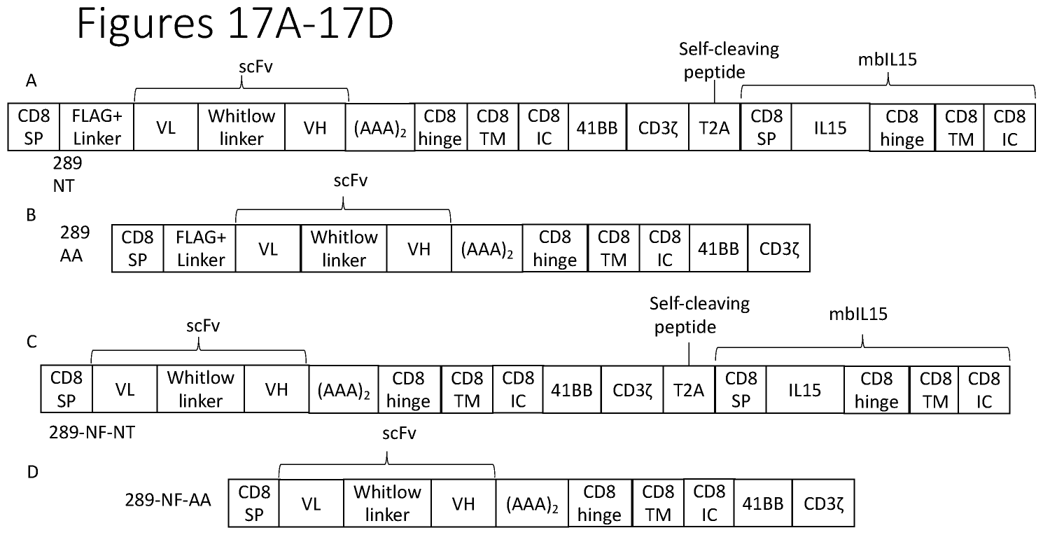

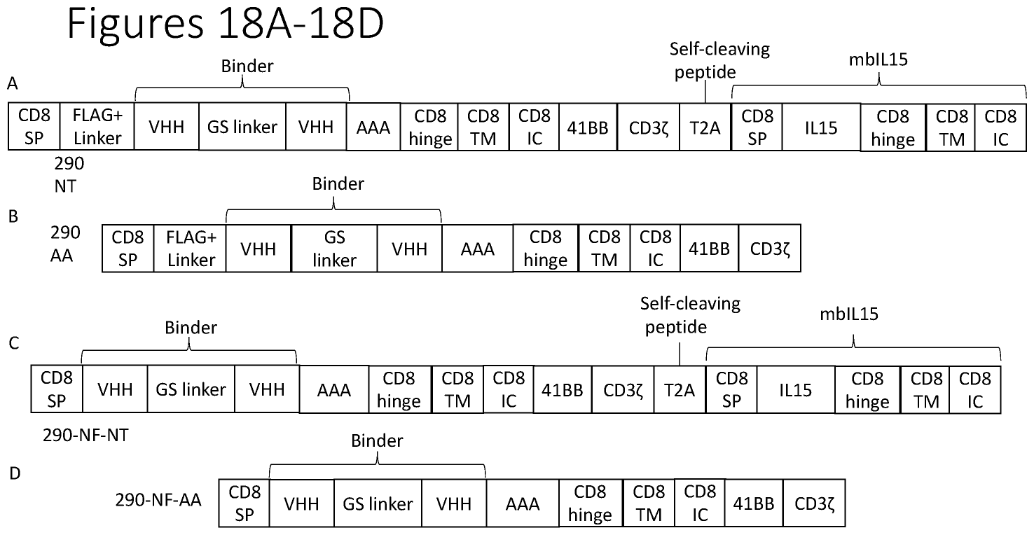

In some embodiments, the VH comprises a sequence having at least 85%, 90%, 91%, 92%, 93%, 94%, 95%, 96%, 97%, 98%, 99%, or 100% identity to any one of SEQ ID NOs: 260-285. In some embodiments, the VL comprises a sequence having at least 85%, 90%, 91%, 92%, 93%, 94%, 95%, 96%, 97%, 98%, 99%, or 100% identity to any one of SEQ ID NOs: 541-566. In some embodiments, the anti-BCMA binding moiety comprises a combination of VH and VL as depicted in FIG. 4.

In some embodiments, the HCDR1 is encoded by a nucleic acid comprising a sequence having at least 85%, 90%, 91%, 92%, 93%, 94%, 95%, 96%, 97%, 98%, 99%, or 100% identity to any one of SEQ ID NOs: 182-207. In some embodiments, the HCDR2 is encoded by a nucleic acid comprising a sequence having at least 85%, 90%, 91%, 92%, 93%, 94%, 95%, 96%, 97%, 98%, 99%, or 100% identity to any one of SEQ ID NOs: 208-233. In some embodiments, the HCDR3 is encoded by a nucleic acid comprising a sequence having at least 85%, 90%, 91%, 92%, 93%, 94%, 95%, 96%, 97%, 98%, 99%, or 100% identity to any one of SEQ ID NOs: 234-259.

In some embodiments, the LCDR1 is encoded by a nucleic acid comprising a sequence having at least 85%, 90%, 91%, 92%, 93%, 94%, 95%, 96%, 97%, 98%, 99%, or 100% identity to any one of SEQ ID NOs: 463-488. In some embodiments, the LCDR2 is encoded by a nucleic acid comprising a sequence having at least 85%, 90%, 91%, 92%, 93%, 94%, 95%, 96%, 97%, 98%, 99%, or 100% identity to any one of SEQ ID NOs: 489-514. In some embodiments, the LCDR3 is encoded by a nucleic acid comprising a sequence having at least 85%, 90%, 91%, 92%, 93%, 94%, 95%, 96%, 97%, 98%, 99%, or 100% identity to any one of SEQ ID NOs: 515-540.

In some embodiments, the VH comprises a sequence having at least 85%, 90%, 91%, 92%, 93%, 94%, 95%, 96%, 97%, 98%, 99%, or 100% identity to any one of SEQ ID NOs: 286-311. In some embodiments, the VH is encoded by a nucleic acid comprising a sequence having at least 85%, 90%, 91%, 92%, 93%, 94%, 95%, 96%, 97%, 98%, 99%, or 100% identity to any one of SEQ ID NOs: 286-311.

In some embodiments, the VL comprises a sequence having at least 85%, 90%, 91%, 92%, 93%, 94%, 95%, 96%, 97%, 98%, 99%, or 100% identity to any one of SEQ ID NOs: 567-592. In some embodiments, the VL is encoded by a nucleic acid comprising a sequence having at least 85%, 90%, 91%, 92%, 93%, 94%, 95%, 96%, 97%, 98%, 99%, or 100% identity to any one of SEQ ID NOs: 567-592.

In some embodiments, the VH further comprises a heavy chain signal peptide (H-SP). In some embodiments, the heavy chain signal peptide comprises the sequence of any one of SEQ ID NOs: 1-9. In some embodiments, the heavy chain signal peptide is encoded by a nucleic acid having the sequence of any one of SEQ ID NOs: 10-19. In some embodiments, the VH further comprises framework regions. In some embodiments, the VH comprises framework regions 1, 2, 3, and 4 (H-FR1, H-FR2, H-FR3, and H-FR4). In some embodiments, H-FR1 comprises the sequence of any one of SEQ ID NOs: 20-32. In some embodiments, H-FR2 comprises the sequence of any one of SEQ ID NOs: 33-38. In some embodiments, H-FR3 comprises the sequence of any one of SEQ ID NOs: 39-53. In some embodiments, H-FR4 comprises the sequence of SEQ ID NO: 54. In some embodiments, H-FR1 is encoded by a nucleic acid having the sequence of any one of SEQ ID NOs: 55-73. In some embodiments, H-FR2 is encoded by a nucleic acid having the sequence of any one of SEQ ID NOs: 74-84. In some embodiments, H-FR3 is encoded by a nucleic acid having the sequence of any one of SEQ ID NOs: 85-102. In some embodiments, H-FR4 is encoded by a nucleic acid having the sequence of SEQ ID NOs: 103.

In some embodiments, the VL further comprises a light chain signal peptide (L-SP). In some embodiments, the light chain signal peptide comprises the sequence of any one of SEQ ID NOs: 312-317. In some embodiments, the light chain signal peptide is encoded by a nucleic acid having the sequence of any one of SEQ ID NOs: 318-324. In some embodiments, the VL further comprises framework regions. In some embodiments, the VL comprises framework regions 1, 2, 3, and 4 (L-FR1, L-FR2, L-FR3, and L-FR4). In some embodiments, the L-FR1 comprises the sequence of any one of SEQ ID NOs: 325-331. In some embodiments, L-FR2 comprises the sequence of any one of SEQ ID NOs: 332-337. In some embodiments, L-FR3 comprises the sequence of any one of SEQ ID NOs: 338-347. In some embodiments, L-FR4 comprises the sequence of any one of SEQ ID NOs: 348-353. In some embodiments, L-FR1 is encoded by a nucleic acid having the sequence of any one of SEQ ID NOs: 354-357 or 1416-1420. In some embodiments, L-FR2 is encoded by a nucleic acid having the sequence of any one of SEQ ID NOs: 358-366. In some embodiments, L-FR3 is encoded by a nucleic acid having the sequence of any one of SEQ ID NOs: 367-377. In some embodiments, L-FR4 is encoded by a nucleic acid having the sequence of any one of SEQ ID NOs: 378-384.

Embodiments of the anti-BCMA binding moieties comprise various arrangements of the VH and VL disclosed herein. In some embodiments, the VH and VL are separated by a linker. In some embodiments, the linker comprises the sequence of SEQ ID NO: 1388.

In some embodiments of the anti-BCMA binding moieties, the VH is N-terminal of the VL. In some embodiments, the anti-BCMA binding moiety comprises a sequence having at least 85%, 90%, 91%, 92%, 93%, 94%, 95%, 96%, 97%, 98%, 99%, or 100% identity to any one of SEQ ID NOs: 593-618. In some embodiments, the anti-BCMA binding moiety is encoded by a nucleic acid comprising a sequence having at least 85%, 90%, 91%, 92%, 93%, 94%, 95%, 96%, 97%, 98%, 99%, or 100% identity to any one of SEQ ID NOs: 619-670.

In some embodiments of the anti-BCMA binding moieties, the VL is N-terminal of the VH. In some embodiments, the anti-BCMA binding moiety comprises a sequence having at least 85%, 90%, 91%, 92%, 93%, 94%, 95%, 96%, 97%, 98%, 99%, or 100% identity to any one of SEQ ID NOs: 671-696. In some embodiments, the anti-BCMA binding moiety is encoded by a nucleic acid comprising a sequence having at least 85%, 90%, 91%, 92%, 93%, 94%, 95%, 96%, 97%, 98%, 99%, or 100% identity to any one of SEQ ID NOs: 697-748.

In some embodiments, the anti-BCMA binding moiety comprises a sequence having at least 85%, 90%, 91%, 92%, 93%, 94%, 95%, 96%, 97%, 98%, 99%, or 100% identity to any one of SEQ ID NOs: 1421-1426. In some embodiments, the anti-BCMA binding moiety is encoded by a nucleic acid comprising a sequence having at least 85%, 90%, 91%, 92%, 93%, 94%, 95%, 96%, 97%, 98%, 99%, or 100% identity to any one of SEQ ID NOs: 1447-1472.

In some embodiments, the anti-BCMA binding moiety comprises a sequence having at least 85%, 90%, 91%, 92%, 93%, 94%, 95%, 96%, 97%, 98%, 99%, or 100% identity to any one of SEQ ID NOs: 1473-1498. In some embodiments, the anti-BCMA binding moiety is encoded by a nucleic acid comprising a sequence having at least 85%, 90%, 91%, 92%, 93%, 94%, 95%, 96%, 97%, 98%, 99%, or 100% identity to any one of SEQ ID NOs: 1499-1524.

In some embodiments, the anti-BCMA binding moiety comprises a sequence having at least 85%, 90%, 91%, 92%, 93%, 94%, 95%, 96%, 97%, 98%, 99%, or 100% identity to any one of SEQ ID NOs: 1582-1600. In some embodiments, the anti-BCMA binding moiety is encoded by a nucleic acid comprising a sequence having at least 85%, 90%, 91%, 92%, 93%, 94%, 95%, 96%, 97%, 98%, 99%, or 100% identity to any one of SEQ ID NOs: 1601-1619.

In some embodiments, the anti-BCMA binding moiety comprises a sequence having at least 85%, 90%, 91%, 92%, 93%, 94%, 95%, 96%, 97%, 98%, 99%, or 100% identity to any one of SEQ ID NOs: 1677-1695. In some embodiments, the anti-BCMA binding moiety is encoded by a nucleic acid comprising a sequence having at least 85%, 90%, 91%, 92%, 93%, 94%, 95%, 96%, 97%, 98%, 99%, or 100% identity to any one of SEQ ID NOs: 1696-1714.

In some embodiments, the anti-BCMA binding moiety comprises a sequence having at least 85%, 90%, 91%, 92%, 93%, 94%, 95%, 96%, 97%, 98%, 99%, or 100% identity to any one of SEQ ID NOs: 1715-1733. In some embodiments, the anti-BCMA binding moiety is encoded by a nucleic acid comprising a sequence having at least 85%, 90%, 91%, 92%, 93%, 94%, 95%, 96%, 97%, 98%, 99%, or 100% identity to any one of SEQ ID NOs: 1734-1771.

In some embodiments, the anti-BCMA binding moiety comprises a sequence having at least 85%, 90%, 91%, 92%, 93%, 94%, 95%, 96%, 97%, 98%, 99%, or 100% identity to any one of SEQ ID NOs: 1772-1790. In some embodiments, the anti-BCMA binding moiety is encoded by a nucleic acid comprising a sequence having at least 85%, 90%, 91%, 92%, 93%, 94%, 95%, 96%, 97%, 98%, 99%, or 100% identity to any one of SEQ ID NOs: 1791-1828.

In some embodiments, the anti-BCMA binding moiety comprises a sequence having at least 85%, 90%, 91%, 92%, 93%, 94%, 95%, 96%, 97%, 98%, 99%, or 100% identity to any one of SEQ ID NOs: 1829-1847. In some embodiments, the anti-BCMA binding moiety is encoded by a nucleic acid comprising a sequence having at least 85%, 90%, 91%, 92%, 93%, 94%, 95%, 96%, 97%, 98%, 99%, or 100% identity to any one of SEQ ID NOs: 1848-1866.

In some embodiments, the anti-BCMA binding moiety comprises a sequence having at least 85%, 90%, 91%, 92%, 93%, 94%, 95%, 96%, 97%, 98%, 99%, or 100% identity to any one of SEQ ID NOs: 1867-1885. In some embodiments, the anti-BCMA binding moiety is encoded by a nucleic acid comprising a sequence having at least 85%, 90%, 91%, 92%, 93%, 94%, 95%, 96%, 97%, 98%, 99%, or 100% identity to any one of SEQ ID NOs: 1886-1923.

In some embodiments, the anti-BCMA binding moiety comprises a sequence having at least 85%, 90%, 91%, 92%, 93%, 94%, 95%, 96%, 97%, 98%, 99%, or 100% identity to any one of SEQ ID NOs: 3255-3277. In some embodiments, the anti-BCMA binding moiety is encoded by a nucleic acid comprising a sequence having at least 85%, 90%, 91%, 92%, 93%, 94%, 95%, 96%, 97%, 98%, 99%, or 100% identity to any one of SEQ ID NOs: 3278-3323.

In some embodiments, the anti-BCMA binding moiety comprises a sequence having at least 85%, 90%, 91%, 92%, 93%, 94%, 95%, 96%, 97%, 98%, 99%, or 100% identity to any one of SEQ ID NOs: 3324-3346. In some embodiments, the anti-BCMA binding moiety is encoded by a nucleic acid comprising a sequence having at least 85%, 90%, 91%, 92%, 93%, 94%, 95%, 96%, 97%, 98%, 99%, or 100% identity to any one of SEQ ID NOs: 3347-3392.

In some embodiments, the anti-BCMA binding moiety comprises a sequence having at least 85%, 90%, 91%, 92%, 93%, 94%, 95%, 96%, 97%, 98%, 99%, or 100% identity to any one of SEQ ID NOs: 5457-5479. In some embodiments, the anti-BCMA binding moiety is encoded by a nucleic acid comprising a sequence having at least 85%, 90%, 91%, 92%, 93%, 94%, 95%, 96%, 97%, 98%, 99%, or 100% identity to any one of SEQ ID NOs: 5480-5502.

In some embodiments, the anti-BCMA binding moiety comprises a sequence having at least 85%, 90%, 91%, 92%, 93%, 94%, 95%, 96%, 97%, 98%, 99%, or 100% identity to any one of SEQ ID NOs: 3859-3862. In some embodiments, the anti-BCMA binding moiety is encoded by a nucleic acid comprising a sequence having at least 85%, 90%, 91%, 92%, 93%, 94%, 95%, 96%, 97%, 98%, 99%, or 100% identity to any one of SEQ ID NOs: 3875-3878.

In some embodiments, the anti-BCMA binding moiety comprises a VHH domain, having a sequence having at least 85%, 90%, 91%, 92%, 93%, 94%, 95%, 96%, 97%, 98%, 99%, or 100% identity to any one of SEQ ID NOs: 5457-5479. In some embodiments, the VHH domain is encoded by a nucleic acid comprising a sequence having at least 85%, 90%, 91%, 92%, 93%, 94%, 95%, 96%, 97%, 98%, 99%, or 100% identity to any one of SEQ ID NOs: 5480-5502.

In some embodiments, the binding moieties provided herein comprise one or more CDR(s) as part of a larger polypeptide chain. In some embodiments, the antigen-binding proteins covalently link the one or more CDR(s) to another polypeptide chain. In some embodiments, the antigen-binding proteins incorporate the one or more CDR(s) noncovalently. In some embodiments, the antigen-binding proteins may comprise at least one of the CDRs described herein incorporated into a biocompatible framework structure. In some embodiments, the biocompatible framework structure comprises a polypeptide or portion thereof that is sufficient to form a conformationally stable structural support, or framework, or scaffold, which is able to display one or more sequences of amino acids that bind to an antigen (e.g., CDRs, a variable region, etc.) in a localized surface region. Such structures can be a naturally occurring polypeptide or polypeptide “fold” (a structural motif), or can have one or more modifications, such as additions, deletions and/or substitutions of amino acids, relative to a naturally occurring polypeptide or fold. Depending on the embodiment, the scaffolds can be derived from a polypeptide of a variety of different species (or of more than one species), such as a human, a non-human primate or other mammal, other vertebrate, invertebrate, plant, bacteria or virus.

Depending on the embodiment, the biocompatible framework structures are based on protein scaffolds or skeletons other than immunoglobulin domains. In some such embodiments, those framework structures are based on fibronectin, ankyrin, lipocalin, neocarzinostain, cytochrome b, CP1 zinc finger, PST1, coiled coil, LACI-D1, Z domain and/or tendamistat domains.

There is also provided, in some embodiments, binding moieties with more than one binding site. In several embodiments, the binding sites are identical to one another while in some embodiments the binding sites are different from one another. For example, an antibody typically has two identical binding sites, while a “bispecific” or “bifunctional” antibody has two different binding sites. The two binding sites of a bispecific antigen-binding protein or antibody will bind to two different epitopes, which can reside on the same or different protein targets. In several embodiments, this is particularly advantageous, as a bispecific chimeric antigen receptor can impart to an engineered cell the ability to target multiple tumor markers, for example, BCMA and an additional tumor marker, such as CD19, CD38, CS1, FCRL5, GPR5CD, CD229, NKG2D or any other marker disclosed herein or appreciated in the art as a tumor specific antigen or tumor associated antigen.

Additional anti-BCMA binding moieties are known in the art, such as those disclosed in, for example, U.S. Pat. Nos. 9,765,342, 10,294,304, 10,174,095, European Patent No. EP 3230321, US Patent Publication No. 2018/0118842, US Patent Publication No. 2019/0153061, and PCT Patent Publication No. WO 2019/149269, the entirety of each of which is incorporated by reference herein.

As used herein, the term “chimeric antibody” shall be given its ordinary meaning, and shall also refer to an antibody that contains one or more regions from one antibody and one or more regions from one or more other antibodies. In some embodiments, one or more of the CDRs are derived from an anti-cancer antigen (e.g., BCMA) antibody. In several embodiments, all of the CDRs are derived from an anti-cancer antigen antibody (such as an anti-BCMA). In some embodiments, the CDRs from more than one anti-cancer antigen antibodies are mixed and matched in a chimeric antibody. For instance, a chimeric antibody may comprise a CDR1 from the light chain of a first anti-cancer antigen antibody, a CDR2 and a CDR3 from the light chain of a second anti-cancer antigen antibody, and the CDRs from the heavy chain from a third anti-cancer antigen antibody. Further, the framework regions of antigen-binding proteins disclosed herein may be derived from one of the same anti-cancer antigen (e.g., BCMA) antibodies, from one or more different antibodies, such as a human antibody, or from a humanized antibody. In one example of a chimeric antibody, a portion of the heavy and/or light chain is identical with, homologous to, or derived from an antibody from a particular species or belonging to a particular antibody class or subclass, while the remainder of the chain(s) is/are identical with, homologous to, or derived from an antibody or antibodies from another species or belonging to another antibody class or subclass. Also provided herein are fragments of such antibodies that exhibit the desired biological activity.

### Cytotoxic Signaling Complex

Some embodiments of the compositions and methods described herein relate to a chimeric antigen receptor, such as a BCMA-directed CAR, that includes a cytotoxic signaling complex. As disclosed herein, according to several embodiments, the provided cytotoxic receptor complexes comprise one or more transmembrane and/or intracellular domains that initiate cytotoxic signaling cascades upon the extracellular domain(s) binding to ligands on the surface of target cells. Certain embodiments disclosed herein relate to chimeric antigen receptor constructs wherein the tumor-targeting domain (e.g., an anti-BCMA binding moiety) is coupled to a cytotoxic signaling complex.

In several embodiments, the cytotoxic signaling complex comprises at least one transmembrane domain, at least one co-stimulatory domain, and/or at least one signaling domain. In some embodiments, more than one component part makes up a given domain—e.g., a co-stimulatory domain may comprise two subdomains. Moreover, in some embodiments, a domain may serve multiple functions, for example, a transmembrane domain may also serve to provide signaling function.

### Hinge Domains

Some embodiments of the CARs disclosed herein, such as the BCMA-directed CARs, comprise a hinge domain. The hinge domain typically serves to separate an extracellular binding moiety from the rest of the CAR components, including the intracellular components that are bridged to the extracellular binding moiety by a transmembrane domain. Any hinge domain disclosed herein or otherwise generally known in the art may be used in the BCMA-directed CARs disclosed herein.

In some embodiments, the hinge domain is a CD8 hinge domain. In some embodiments, the CD8 hinge domain comprises a sequence having at least 85%, 90%, 91%, 92%, 93%, 94%, 95%, 96%, 97%, 98%, 99%, or 100% identity to SEQ ID NO: 1389. In some embodiments, the CD8 hinge domain is encoded by a nucleic acid comprising a sequence having at least 85%, 90%, 91%, 92%, 93%, 94%, 95%, 96%, 97%, 98%, 99%, or 100% identity to SEQ ID NO: 1400. In some embodiments, the CD8 hinge domain is truncated or modified and is at least 70%, at least 75%, at least 80%, at least 85%, at least 90%, at least 95% homologous with the peptide having the sequence of SEQ ID NO: 1389.

In some embodiments, the hinge domain is an IgG4 hinge domain. In some embodiments, the IgG4 hinge domain comprises a sequence having at least 85%, 90%, 91%, 92%, 93%, 94%, 95%, 96%, 97%, 98%, 99%, or 100% identity to SEQ ID NO: 1390. In some embodiments, the IgG4 hinge domain is encoded by a nucleic acid comprising a sequence having at least 85%, 90%, 91%, 92%, 93%, 94%, 95%, 96%, 97%, 98%, 99%, or 100% identity to SEQ ID NO: 1401. In some embodiments, the IgG4 hinge domain is truncated or modified and is at least 70%, at least 75%, at least 80%, at least 85%, at least 90%, at least 95% homologous with the peptide having the sequence of SEQ ID NO: 1390.

In some embodiments, the hinge domain is an RQRCD8 hinge domain. As understood in the art, the “RQRCD8 hinge domain” is a CD8 hinge domain with additional CD20 and CD34 epitopes. The presence of these additional epitopes permit alternative methods of detection with these epitopes, as well as enabling the selected depletion of immune cells engineered with CARs having these epitopes (e.g. using antibodies specific for CD20 or CD34). In some embodiments, the RQRCD8 hinge domain comprises a sequence having at least 85%, 90%, 91%, 92%, 93%, 94%, 95%, 96%, 97%, 98%, 99%, or 100% identity to SEQ ID NO: 1391. In some embodiments, the RQRCD8 hinge domain is encoded by a nucleic acid comprising a sequence having at least 85%, 90%, 91%, 92%, 93%, 94%, 95%, 96%, 97%, 98%, 99%, or 100% identity to SEQ ID NO: 1402. In some embodiments, the RQRCD8 hinge domain is truncated or modified and is at least 70%, at least 75%, at least 80%, at least 85%, at least 90%, at least 95% homologous with the peptide having the sequence of SEQ ID NO: 1391. The RQRCD8 hinge domain (also referred as “RQR8”) is explored in Philip et al. “A highly compact epitope-based marker/suicide gene for easier and safer T-cell therapy” Blood 124(8):1277-1287 and PCT publication WO 2013/153391, each of which is hereby expressly incorporated by reference in its entirety.

### Transmembrane Domains

Some embodiments of the CARs disclosed herein, such as the BCMA-directed CARs, comprise a transmembrane domain. As conventionally understood, the transmembrane domain serves to act as the region spanning the plasma membrane of a cell to connect the extracellular and intracellular domains of the CAR. Any transmembrane domain disclosed herein or otherwise generally known in the art may be used in the BCMA-directed CARs disclosed herein.

In some embodiments, the transmembrane domain is a CD8 transmembrane domain (CD8TM). In some embodiments, the CD8 transmembrane domain comprises a sequence having at least 85%, 90%, 91%, 92%, 93%, 94%, 95%, 96%, 97%, 98%, 99%, or 100% identity to SEQ ID NO: 1392. In some embodiments, the CD8 transmembrane domain is encoded by a nucleic acid comprising a sequence having at least 85%, 90%, 91%, 92%, 93%, 94%, 95%, 96%, 97%, 98%, 99%, or 100% identity to SEQ ID NO: 1403. In some embodiments, the CD8 transmembrane domain is truncated or modified and is at least 70%, at least 75%, at least 80%, at least 85%, at least 90%, at least 95% homologous with the peptide having the sequence of SEQ ID NO: 1392.

In some embodiments, the CD8 transmembrane domain may also comprise a CD8 intracellular domain (CD8IC). In some embodiments, the CD8 intracellular domain comprises the sequence of SEQ ID NO: 1393. In some embodiments, the CD8 intracellular domain is encoded by a nucleic acid comprising the sequence of SEQ ID NO: 1404. In some embodiments, for the purposes for this disclosure, the CD8 transmembrane domain can be considered to include the CD8 intracellular domain, such that the CD8 transmembrane domain comprises the sequences of SEQ ID NO: 1392 and SEQ ID NO: 1393 from N-terminal to C-terminal orientation.

In some embodiments, the transmembrane domain is CD28 transmembrane domain (CD28TM). In some embodiments, the CD28 transmembrane domain comprises a sequence having at least 85%, 90%, 91%, 92%, 93%, 94%, 95%, 96%, 97%, 98%, 99%, or 100% identity to SEQ ID NO: 1414. In some embodiments, the CD8 transmembrane domain is encoded by a nucleic acid comprising a sequence having at least 85%, 90%, 91%, 92%, 93%, 94%, 95%, 96%, 97%, 98%, 99%, or 100% identity to SEQ ID NO: 1415. In some embodiments, the CD8 transmembrane domain is truncated or modified and is at least 70%, at least 75%, at least 80%, at least 85%, at least 90%, at least 95% homologous with the peptide having the sequence of SEQ ID NO: 1414.

### Signaling Domains

Some embodiments of the CARs disclosed herein, such as the BCMA-directed CARs, comprise an intracellular signaling domain. As generally understood in the art, unexpectedly enhanced signaling can be achieved through the use of multiple signaling domains whose activities act synergistically. Any intracellular signaling domain disclosed herein or otherwise generally known in the art may be used in the BCMA-directed CARs disclosed herein.

In some embodiments, the intracellular signaling domain comprises an OX40 subdomain and a CD3ζ subdomain. In some embodiments, the OX40 subdomain comprises a sequence having at least 85%, 90%, 91%, 92%, 93%, 94%, 95%, 96%, 97%, 98%, 99%, or 100% identity to SEQ ID NO: 1394. In some embodiments, the OX40 subdomain is encoded by a nucleic acid comprising a sequence having at least 85%, 90%, 91%, 92%, 93%, 94%, 95%, 96%, 97%, 98%, 99%, or 100% identity to SEQ ID NO: 1405. In some embodiments, the OX40 subdomain is truncated or modified and is at least 70%, at least 75%, at least 80%, at least 85%, at least 90%, at least 95% homologous with the peptide having the sequence of SEQ ID NO: 1394. In some embodiments, the CD3ζ subdomain comprises a sequence having at least 85%, 90%, 91%, 92%, 93%, 94%, 95%, 96%, 97%, 98%, 99%, or 100% identity to SEQ ID NO: 1395. In some embodiments, the CD3ζ subdomain is encoded by a nucleic acid comprising a sequence having at least 85%, 90%, 91%, 92%, 93%, 94%, 95%, 96%, 97%, 98%, 99%, or 100% identity to SEQ ID NO: 1406. In some embodiments, the CD3ζ subdomain is truncated or modified and is at least 70%, at least 75%, at least 80%, at least 85%, at least 90%, at least 95% homologous with the peptide having the sequence of SEQ ID NO: 1395. In some embodiments, the OX40 subdomain is N-terminal to the CD3ζ subdomain. In some embodiments, the CD3ζ subdomain is N-terminal to the OX40 subdomain.

### Co-Stimulatory Domains

In some embodiments, the CARs disclosed herein are expressed along with a co-stimulatory domain, where additional co-activating molecules improve aspects of CAR activity, including but not limited to cytotoxic activity, CAR stability, and cell longevity. In some embodiments, the additional co-activating molecules can be cytokines, including but not limited to certain interleukins, such as interleukin 2 (IL2) and/or interleukin 15 (IL15). In some embodiments, the immune cells expressing the CAR are also engineered to express such additional co-activating molecules as a secreted form. In some embodiments, the immune cells expressing the CAR are also engineered to express such additional co-activating molecules as a membrane bound form, acting as autocrine stimulatory molecules (or even as paracrine stimulators to neighboring cells). Membrane-bound IL15 is explored in WO 2015/174928, which is hereby expressly incorporated by reference in its entirety.

In some embodiments, the immune cells are engineered to express IL15, optionally as a membrane-bound IL15 (mbIL15). In such embodiments, mbIL15 expression on the immune cell enhances the cytotoxic effects of the engineered immune cells by enhancing the proliferation and/or longevity of the immune cells. In some embodiments, the IL15 is human IL15. In some embodiments, the IL15 comprises the sequence of SEQ ID NO: 1397. In some embodiments, the IL15 is encoded by a nucleic acid comprising the sequence of SEQ ID NO: 1408. In some embodiments, the mbIL15 is assembled by fusing an IL15 with one or more of a CD8 signal peptide (CD8SP), CD8 hinge (CD8h), CD8 transmembrane domain (CD8TM), and a CD8 intracellular domain (CD8IC). In some embodiments, the mbIL15 is assembled according to the order: CD8SP-IL15-CD8h-CD8TM-CD8IC, from N-terminus to C-terminus. However, other methods of producing a membrane-bound IL15 are also envisioned.

In some embodiments, the CD8SP of the mbIL15 comprises the sequence of SEQ ID NO: 1396. In some embodiments, the CD8h of the mbIL15 comprises the sequence of SEQ ID NO: 1400. In some embodiments, the CD8TM of the mbIL15 comprises the sequence of SEQ ID NO: 1392. In some embodiments, the CD8IC of the mbIL15 comprises the sequence of SEQ ID NO: 1393. In some embodiments, the CD8SP of the mbIL15 is encoded by a nucleic acid comprising the sequence of SEQ ID NO: 1407. In some embodiments, the CD8h of the mbIL15 is encoded by a nucleic acid comprising the sequence of SEQ ID NO: 1409. In some embodiments, the CD8TM of the mbIL15 is encoded by a nucleic acid comprising the sequence of SEQ ID NO: 1410. In some embodiments, the CD8IC of the mbIL15 is encoded by a nucleic acid comprising the sequence of SEQ ID NO: 1411. In some embodiments, the mbIL15 comprises a sequence having at least 85%, 90%, 91%, 92%, 93%, 94%, 95%, 96%, 97%, 98%, 99%, or 100% identity to the sequence of SEQ ID NO: 1398. In some embodiments, the mbIL15 is encoded by a nucleic acid comprising a sequence having at least 85%, 90%, 91%, 92%, 93%, 94%, 95%, 96%, 97%, 98%, 99%, or 100% identity to the sequence of SEQ ID NO: 1412. In some embodiments, the mbIL15 can be truncated or modified, such that it has at least 70%, at least 75%, at least 80%, at least 85%, at least 90%, at least 95% sequence identity with the sequence of SEQ ID NO: 1398.

In some embodiments, the CAR and mbIL15 are expressed simultaneously in a bicistronic configuration as a polypeptide comprising a self-cleaving peptide. In some embodiments, the CAR and mbIL15 are expressed simultaneously in a bicistronic configuration from a polynucleotide comprising a sequence encoding for a self-cleaving peptide. In some embodiments, the self-cleaving peptide is a T2A self-cleaving peptide, a P2A self-cleaving peptide, an E2A self-cleaving peptide, or an F2A self-cleaving peptide. In this way, the CAR and mbIL15 can be delivered to an immune cell as a single vector, if needed. In some embodiments, a T2A self-cleaving peptide is used. In some embodiments, the T2A self-cleaving peptide comprises the sequence of SEQ ID NO: 1399. In some embodiments, the T2A self-cleaving peptide is encoded by a nucleic acid comprising the sequence of SEQ ID NO: 1413.

### BCMA-Directed Chimeric Antigen Receptors

Disclosed herein are BCMA-directed CARs. In some embodiments, the BCMA-directed CARs comprise an extracellular anti-BCMA binding moiety, a hinge domain, a transmembrane domain, and an intracellular signaling domain comprising an OX40 subdomain and a CD3ζ subdomain. In some embodiments, the anti-BCMA binding moiety may be any one of the anti-BCMA binding moieties disclosed herein. In some embodiments, the hinge domain may be any one of the hinge domains disclosed herein. In some embodiments, the transmembrane domain may be any one of the transmembrane domains disclosed herein. In some embodiments, the intracellular signaling domain may be any one of the intracellular signaling domains disclosed herein.

In some embodiments, the OX40 subdomain (OX40) comprises a sequence having at least 85%, 90%, 91%, 92%, 93%, 94%, 95%, 96%, 97%, 98%, 99%, or 100% identity to SEQ ID NO: 1394. In some embodiments, the OX40 subdomain is encoded by a nucleic acid comprising a sequence having at least 85%, 90%, 91%, 92%, 93%, 94%, 95%, 96%, 97%, 98%, 99%, or 100% identity to SEQ ID NO: 1405. In some embodiments, the OX40 subdomain is truncated or modified and is at least 70%, at least 75%, at least 80%, at least 85%, at least 90%, at least 95% homologous with the peptide having the sequence of SEQ ID NO: 1394. In some embodiments, the CD3ζ subdomain comprises a sequence having at least 85%, 90%, 91%, 92%, 93%, 94%, 95%, 96%, 97%, 98%, 99%, or 100% identity to SEQ ID NO: 1395. In some embodiments, the CD3ζ subdomain is encoded by a nucleic acid comprising a sequence having at least 85%, 90%, 91%, 92%, 93%, 94%, 95%, 96%, 97%, 98%, 99%, or 100% identity to SEQ ID NO: 1406. In some embodiments, the CD3ζ subdomain is truncated or modified and is at least 70%, at least 75%, at least 80%, at least 85%, at least 90%, at least 95% homologous with the peptide having the sequence of SEQ ID NO: 1395. In some embodiments, the OX40 subdomain is N-terminal to the CD3ζ subdomain. In some embodiments, the CD3ζ subdomain is N-terminal to the OX40 subdomain.

In some embodiments, the transmembrane domain is a CD8 transmembrane domain (CD8TM). In some embodiments, the CD8 transmembrane domain comprises a sequence having at least 85%, 90%, 91%, 92%, 93%, 94%, 95%, 96%, 97%, 98%, 99%, or 100% identity to SEQ ID NO: 1392. In some embodiments, the CD8 transmembrane domain is encoded by a nucleic acid comprising a sequence having at least 85%, 90%, 91%, 92%, 93%, 94%, 95%, 96%, 97%, 98%, 99%, or 100% identity to SEQ ID NO: 1403. In some embodiments, the CD8 transmembrane domain is truncated or modified and is at least 70%, at least 75%, at least 80%, at least 85%, at least 90%, at least 95% homologous with the peptide having the sequence of SEQ ID NO: 1392.

In some embodiments, the transmembrane domain is CD28 transmembrane domain (CD28TM). In some embodiments, the CD28 transmembrane domain comprises a sequence having at least 85%, 90%, 91%, 92%, 93%, 94%, 95%, 96%, 97%, 98%, 99%, or 100% identity to SEQ ID NO: 1414. In some embodiments, the CD8 transmembrane domain is encoded by a nucleic acid comprising a sequence having at least 85%, 90%, 91%, 92%, 93%, 94%, 95%, 96%, 97%, 98%, 99%, or 100% identity to SEQ ID NO: 1415. In some embodiments, the CD8 transmembrane domain is truncated or modified and is at least 70%, at least 75%, at least 80%, at least 85%, at least 90%, at least 95% homologous with the peptide having the sequence of SEQ ID NO: 1414.

In some embodiments, the hinge domain is a CD8 hinge domain (CD8h). In some embodiments, the CD8 hinge domain comprises a sequence having at least 85%, 90%, 91%, 92%, 93%, 94%, 95%, 96%, 97%, 98%, 99%, or 100% identity to SEQ ID NO: 1389. In some embodiments, the CD8 hinge domain is encoded by a nucleic acid comprising a sequence having at least 85%, 90%, 91%, 92%, 93%, 94%, 95%, 96%, 97%, 98%, 99%, or 100% identity to SEQ ID NO: 1400. In some embodiments, the CD8 hinge domain is truncated or modified and is at least 70%, at least 75%, at least 80%, at least 85%, at least 90%, at least 95% homologous with the peptide having the sequence of SEQ ID NO: 1389.

In some embodiments, the CD8 hinge domain, transmembrane domain, and intracellular signaling domain make up the part of the BCMA-directed CAR other than the anti-BCMA binding moiety. In some embodiments, the BCMA-directed CAR comprises a CD8 hinge domain (CD8h), a CD8 transmembrane domain (CD8TM), a CD8 intracellular domain (CD8IC), an OX40 subdomain (OX40), and a CD3ζ subdomain (CD3ζ). In some embodiments, the CD8h, CD8TM, CD8IC, OX40, and CD3ζ are in the order of CD8h-CD8TM-CD8IC-OX40-CD3ζ, from N-terminus to C-terminus. In some embodiments, the CD8 hinge domain, transmembrane domain, and intracellular signaling domain are represented by a sequence having at least 85%, 90%, 91%, 92%, 93%, 94%, 95%, 96%, 97%, 98%, 99%, or 100% identity to SEQ ID NO: 1375. In some embodiments, the CD8 hinge domain, transmembrane domain, and intracellular signaling domain are encoded by a nucleic acid comprising a sequence having at least 85%, 90%, 91%, 92%, 93%, 94%, 95%, 96%, 97%, 98%, 99%, or 100% identity to SEQ ID NO: 1376.

In some embodiments where the BCMA-directed CAR comprises a CD8h, CD8TM, CD8IC, OX40, and CD3ζ with the anti-BCMA binding moiety, the BCMA-directed CAR may comprise a sequence having at least 85%, 90%, 91%, 92%, 93%, 94%, 95%, 96%, 97%, 98%, 99%, or 100% identity to any one of SEQ ID NOs: 749-774. In some embodiments, the BCMA-directed CAR is encoded by a nucleic acid having a sequence at least 85%, 90%, 91%, 92%, 93%, 94%, 95%, 96%, 97%, 98%, 99%, or 100% identity to any one of SEQ ID NOs: 775-800. In some embodiments, the BCMA-directed CAR is encoded by a nucleic acid codon optimized for human and having a sequence at least 85%, 90%, 91%, 92%, 93%, 94%, 95%, 96%, 97%, 98%, 99%, or 100% identity to any one of SEQ ID NOs: 801-826. In some embodiments, the BCMA-directed CAR comprises a sequence having at least 85%, 90%, 91%, 92%, 93%, 94%, 95%, 96%, 97%, 98%, 99%, or 100% identity to any one of SEQ ID NOs: 905-930. In some embodiments, the BCMA-directed CAR is encoded by a nucleic acid having a sequence at least 85%, 90%, 91%, 92%, 93%, 94%, 95%, 96%, 97%, 98%, 99%, or 100% identity to any one of SEQ ID NOs: 931-956. In some embodiments, the BCMA-directed CAR is encoded by a nucleic acid codon optimized for human and having a sequence at least 85%, 90%, 91%, 92%, 93%, 94%, 95%, 96%, 97%, 98%, 99%, or 100% identity to any one of SEQ ID NOs: 957-982.

In some embodiments, the hinge domain is an IgG4 hinge domain. In some embodiments, the IgG4 hinge domain comprises a sequence having at least 85%, 90%, 91%, 92%, 93%, 94%, 95%, 96%, 97%, 98%, 99%, or 100% identity to SEQ ID NO: 1390. In some embodiments, the IgG4 hinge domain is encoded by a nucleic acid comprising a sequence having at least 85%, 90%, 91%, 92%, 93%, 94%, 95%, 96%, 97%, 98%, 99%, or 100% identity to SEQ ID NO: 1401. In some embodiments, the IgG4 hinge domain is truncated or modified and is at least 70%, at least 75%, at least 80%, at least 85%, at least 90%, at least 95% homologous with the peptide having the sequence of SEQ ID NO: 1390.

In some embodiments, the IgG4 hinge domain, transmembrane domain, and intracellular signaling domain make up the part of the BCMA-directed CAR other than the anti-BCMA binding moiety. In some embodiments, the BCMA-directed CAR comprises an IgG4 hinge domain (IgG4h), a CD8TM, a CD8IC, an OX40, and a CD3ζ. In some embodiments, the IgG4h, CD8TM, CD8IC, OX40, and CD3ζ are in the order of IgG4h-CD8TM-CD8IC-OX40-CD3ζ, from N-terminus to C-terminus. In some embodiments, the IgG4 hinge domain, transmembrane domain, and intracellular signaling domain are represented by a sequence having at least 85%, 90%, 91%, 92%, 93%, 94%, 95%, 96%, 97%, 98%, 99%, or 100% identity to SEQ ID NO: 1377. In some embodiments, the IgG4 hinge domain, transmembrane domain, and intracellular signaling domain are encoded by a nucleic acid comprising a sequence having at least 85%, 90%, 91%, 92%, 93%, 94%, 95%, 96%, 97%, 98%, 99%, or 100% identity to SEQ ID NO: 1378.

In some embodiments where the BCMA-directed CAR comprises an IgG4, CD8TM, CD8IC, OX40, and CD3ζ with the anti-BCMA binding moiety, the BCMA-directed CAR may comprise a sequence having at least 85%, 90%, 91%, 92%, 93%, 94%, 95%, 96%, 97%, 98%, 99%, or 100% identity to any one of SEQ ID NOs: 1061-1086. In some embodiments, the BCMA-directed CAR is encoded by a nucleic acid having a sequence at least 85%, 90%, 91%, 92%, 93%, 94%, 95%, 96%, 97%, 98%, 99%, or 100% identity to any one of SEQ ID NOs: 1087-1112. In some embodiments, the BCMA-directed CAR is encoded by a nucleic acid codon optimized for human and having a sequence at least 85%, 90%, 91%, 92%, 93%, 94%, 95%, 96%, 97%, 98%, 99%, or 100% identity to any one of SEQ ID NOs: 1113-1138.

In some embodiments, the hinge domain is an RQRCD8 hinge domain. In some embodiments, the RQRCD8 hinge domain comprises a sequence having at least 85%, 90%, 91%, 92%, 93%, 94%, 95%, 96%, 97%, 98%, 99%, or 100% identity to SEQ ID NO: 1391. In some embodiments, the RQRCD8 hinge domain is encoded by a nucleic acid comprising a sequence having at least 85%, 90%, 91%, 92%, 93%, 94%, 95%, 96%, 97%, 98%, 99%, or 100% identity to SEQ ID NO: 1402. In some embodiments, the RQRCD8 hinge domain is truncated or modified and is at least 70%, at least 75%, at least 80%, at least 85%, at least 90%, at least 95% homologous with the peptide having the sequence of SEQ ID NO: 1391.

In some embodiments, the RQRCD8 hinge domain, transmembrane domain, and intracellular signaling domain make up the part of the BCMA-directed CAR other than the anti-BCMA binding moiety. In some embodiments, the BCMA-directed CAR comprises an RQRCD8 hinge domain (RQRCD8h), a CD8TM, a CD8IC, an OX40, and a CD3ζ. In some embodiments, the RQRCD8h, CD8TM, CD8IC, OX40, and CD3ζ are in the order of RQRCD8h-CD8TM-CD8IC-OX40-CD3ζ, from N-terminus to C-terminus. In some embodiments, the RQRCD8 hinge domain, transmembrane domain, and intracellular signaling domain are represented by a sequence having at least 85%, 90%, 91%, 92%, 93%, 94%, 95%, 96%, 97%, 98%, 99%, or 100% identity to SEQ ID NO: 1379. In some embodiments, the RQRCD8 hinge domain, transmembrane domain, and intracellular signaling domain are encoded by a nucleic acid comprising a sequence having at least 85%, 90%, 91%, 92%, 93%, 94%, 95%, 96%, 97%, 98%, 99%, or 100% identity to SEQ ID NO: 1380.

In some embodiments where the BCMA-directed CAR comprises an RQRCD8, CD8TM, CD8IC, OX40, and CD3ζ with the anti-BCMA binding moiety, the BCMA-directed CAR may comprise a sequence having at least 85%, 90%, 91%, 92%, 93%, 94%, 95%, 96%, 97%, 98%, 99%, or 100% identity to any one of SEQ ID NOs: 1217-1242. In some embodiments, the BCMA-directed CAR is encoded by a nucleic acid having a sequence at least 85%, 90%, 91%, 92%, 93%, 94%, 95%, 96%, 97%, 98%, 99%, or 100% identity to any one of SEQ ID NOs: 1243-1268. In some embodiments, the BCMA-directed CAR is encoded by a nucleic acid codon optimized for human and having a sequence at least 85%, 90%, 91%, 92%, 93%, 94%, 95%, 96%, 97%, 98%, 99%, or 100% identity to any one of SEQ ID NOs: 1269-1294.

### BCMA-Directed Chimeric Antigen Receptor Constructs

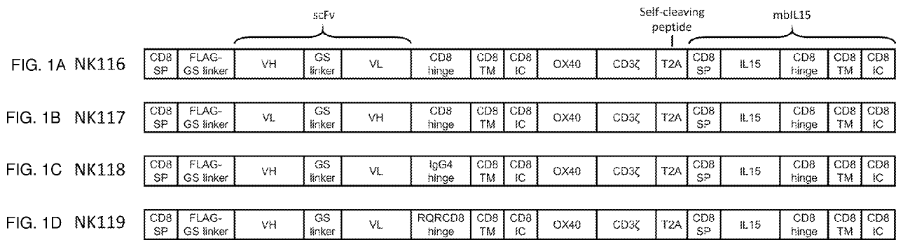

In some embodiments, BCMA-directed CAR constructs comprising a BCMA-directed CAR are provided. FIG. 1 depicts exemplary BCMA-directed CAR constructs as embodied in the present disclosure. As shown in FIG. 1, embodiments of BCMA-directed CAR constructs comprise an anti-BCMA binding moiety, a hinge domain, a CD8 transmembrane domain, a CD8 intracellular domain, an OX40 subdomain, a CD3ζ subdomain, a T2A self-cleaving peptide, and a membrane-bound IL15. In some embodiments, the CD8 transmembrane domain may be substituted for a CD28 transmembrane domain. Any one or more of these components may be any of the embodiments of the respective components disclosed herein. In some embodiments, the BCMA-directed CAR is any one of the BCMA-directed CARs disclosed herein, and the BCMA-directed CAR constructs comprise said BCMA-directed CARs and a membrane-bound IL15 in a bicistronic configuration.

In some embodiments, the BCMA-directed CAR construct comprises a BCMA-directed CAR comprising an anti-BCMA binding moiety, a CD8h, a CD8TM, a CD8IC, an OX40, and a CD3ζ and a membrane-bound IL15 (mbIL15), where the BCMA-directed CAR and mbIL15 are separated by a self-cleaving peptide (e.g. a T2A self-cleaving peptide). In some embodiments, the BCMA-directed CAR construct is arranged in the order of the anti-BCMA binding moiety-CD8h-CD8TM-CD8IC-OX40-CD3ζ-T2A-mbIL15, from N-terminus to C-terminus. In some embodiments, the CD8h-CD8TM-CD8IC-OX40-CD3ζ-T2A-mbIL15 comprises a sequence having at least 85%, 90%, 91%, 92%, 93%, 94%, 95%, 96%, 97%, 98%, 99%, or 100% identity to SEQ ID NO: 1381. In some embodiments, the CD8h-CD8TM-CD8IC-OX40-CD3ζ-T2A-mbIL15 is encoded by a nucleic acid comprising a sequence having at least 85%, 90%, 91%, 92%, 93%, 94%, 95%, 96%, 97%, 98%, 99%, or 100% identity to SEQ ID NO: 1382. In some embodiments, the anti-BCMA binding moiety comprises a VH and VL arranged such that the VH is N-terminal to the VL. In some embodiments, the BCMA-directed CAR construct comprises a sequence having at least 85%, 90%, 91%, 92%, 93%, 94%, 95%, 96%, 97%, 98%, 99%, or 100% identity to any one of SEQ ID NOs: 827-852. In some embodiments, the BCMA-directed CAR construct is encoded by a nucleic acid comprising a sequence having at least 85%, 90%, 91%, 92%, 93%, 94%, 95%, 96%, 97%, 98%, 99%, or 100% identity to any one of SEQ ID NOs: 853-878. In some embodiments, the BCMA-directed CAR construct is encoded by a nucleic acid codon optimized for human and comprising a sequence having at least 85%, 90%, 91%, 92%, 93%, 94%, 95%, 96%, 97%, 98%, 99%, or 100% identity to any one of SEQ ID NOs: 879-904.

In some embodiments, the BCMA-directed CAR construct comprises a BCMA-directed CAR comprising an anti-BCMA binding moiety, a CD8h, a CD8TM, a CD8IC, an OX40, and a CD3ζ and an mbIL15, where the BCMA-directed CAR and mbIL15 are separated by a self-cleaving peptide (e.g. a T2A self-cleaving peptide). In some embodiments, the BCMA-directed CAR construct is arranged in the order of the anti-BCMA binding moiety-CD8h-CD8TM-CD8IC-OX40-CD3ζ-T2A-mbIL15, from N-terminus to C-terminus. In some embodiments, the CD8h-CD8TM-CD8IC-OX40-CD3ζ-T2A-mbIL15 comprises a sequence having at least 85%, 90%, 91%, 92%, 93%, 94%, 95%, 96%, 97%, 98%, 99%, or 100% identity to SEQ ID NO: 1381. In some embodiments, the CD8h-CD8TM-CD8IC-OX40-CD3ζ-T2A-mbIL15 is encoded by a nucleic acid comprising a sequence having at least 85%, 90%, 91%, 92%, 93%, 94%, 95%, 96%, 97%, 98%, 99%, or 100% identity to SEQ ID NO: 1382. In some embodiments, the anti-BCMA binding moiety comprises a VH and VL arranged such that the VL is N-terminal to the VH. In some embodiments, the BCMA-directed CAR construct comprises a sequence having at least 85%, 90%, 91%, 92%, 93%, 94%, 95%, 96%, 97%, 98%, 99%, or 100% identity to any one of SEQ ID NOs: 983-1008. In some embodiments, the BCMA-directed CAR construct is encoded by a nucleic acid comprising a sequence having at least 85%, 90%, 91%, 92%, 93%, 94%, 95%, 96%, 97%, 98%, 99%, or 100% identity to any one of SEQ ID NOs: 1009-1034. In some embodiments, the BCMA-directed CAR construct is encoded by a nucleic acid codon optimized for human and comprising a sequence having at least 85%, 90%, 91%, 92%, 93%, 94%, 95%, 96%, 97%, 98%, 99%, or 100% identity to any one of SEQ ID NOs: 1035-1060.

In some embodiments, the BCMA-directed CAR construct comprises a BCMA-directed CAR comprising an anti-BCMA binding moiety, an IgG4h, a CD8TM, a CD8IC, an OX40, and a CD3ζ and an mbIL15, where the BCMA-directed CAR and mbIL15 are separated by a self-cleaving peptide (e.g. a T2A self-cleaving peptide). In some embodiments, the BCMA-directed CAR construct is arranged in the order of the anti-BCMA binding moiety-IgG4h-CD8TM-CD8IC-OX40-CD3ζ-T2A-mbIL15, from N-terminus to C-terminus. In some embodiments, the IgG4h-CD8TM-CD8IC-OX40-CD3ζ-T2A-mbIL15 comprises a sequence having at least 85%, 90%, 91%, 92%, 93%, 94%, 95%, 96%, 97%, 98%, 99%, or 100% identity to SEQ ID NO: 1383. In some embodiments, the IgG4h-CD8TM-CD8IC-OX40-CD3ζ-T2A-mbIL15 is encoded by a nucleic acid comprising a sequence having at least 85%, 90%, 91%, 92%, 93%, 94%, 95%, 96%, 97%, 98%, 99%, or 100% identity to SEQ ID NO: 1384. In some embodiments, the BCMA-directed CAR construct comprises a sequence having at least 85%, 90%, 91%, 92%, 93%, 94%, 95%, 96%, 97%, 98%, 99%, or 100% identity to any one of SEQ ID NOs: 1139-1164. In some embodiments, the BCMA-directed CAR construct is encoded by a nucleic acid comprising a sequence having at least 85%, 90%, 91%, 92%, 93%, 94%, 95%, 96%, 97%, 98%, 99%, or 100% identity to any one of SEQ ID NOs: 1165-1190. In some embodiments, the BCMA-directed CAR construct is encoded by a nucleic acid codon optimized for human and comprising a sequence having at least 85%, 90%, 91%, 92%, 93%, 94%, 95%, 96%, 97%, 98%, 99%, or 100% identity to any one of SEQ ID NOs: 1191-1216.

In some embodiments, the BCMA-directed CAR construct comprises a BCMA-directed CAR comprising an anti-BCMA binding moiety, an RQR8CDh, a CD8TM, a CD8IC, an OX40, and a CD3ζ and an mbIL15, where the BCMA-directed CAR and mbIL15 are separated by a self-cleaving peptide (e.g. a T2A self-cleaving peptide). In some embodiments, the BCMA-directed CAR construct is arranged in the order of the anti-BCMA binding moiety-RQR8CDh-CD8TM-CD8IC-OX40-CD3ζ-T2A-mbIL15, from N-terminus to C-terminus. In some embodiments, the RQRCD8h-CD8TM-CD8IC-OX40-CD3ζ-T2A-mbIL15 comprises a sequence having at least 85%, 90%, 91%, 92%, 93%, 94%, 95%, 96%, 97%, 98%, 99%, or 100% identity to SEQ ID NO: 1385. In some embodiments, the RQRCD8h-CD8TM-CD8IC-OX40-CD3ζ-T2A-mbIL15 is encoded by a nucleic acid comprising a sequence having at least 85%, 90%, 91%, 92%, 93%, 94%, 95%, 96%, 97%, 98%, 99%, or 100% identity to SEQ ID NO: 1386. In some embodiments, the BCMA-directed CAR construct comprises a sequence having at least 85%, 90%, 91%, 92%, 93%, 94%, 95%, 96%, 97%, 98%, 99%, or 100% identity to any one of SEQ ID NOs: 1295-1320. In some embodiments, the BCMA-directed CAR construct is encoded by a nucleic acid comprising a sequence having at least 85%, 90%, 91%, 92%, 93%, 94%, 95%, 96%, 97%, 98%, 99%, or 100% identity to any one of SEQ ID NOs: 1321-1346. In some embodiments, the BCMA-directed CAR construct is encoded by a nucleic acid codon optimized for human and comprising a sequence having at least 85%, 90%, 91%, 92%, 93%, 94%, 95%, 96%, 97%, 98%, 99%, or 100% identity to any one of SEQ ID NOs: 1347-1372.

In some embodiments, the BCMA-directed CAR is encoded by a nucleic acid that also encodes mbIL15 and has at least 85%, 90%, 91%, 92%, 93%, 94%, 95%, 96%, 97%, 98%, 99%, or 100% identity to any one of SEQ ID NOs: 3851-3854.

In some embodiments, the BCMA-directed CAR comprises a sequence having at least 85%, 90%, 91%, 92%, 93%, 94%, 95%, 96%, 97%, 98%, 99%, or 100% identity to any one of SEQ ID NOs: 3867-3870. In some embodiments, the BCMA-directed CAR is encoded by a nucleic acid comprising a sequence having at least 85%, 90%, 91%, 92%, 93%, 94%, 95%, 96%, 97%, 98%, 99%, or 100% identity to any one of SEQ ID NOs: 3883-3886.

In some embodiments, the BCMA-directed CAR comprises a sequence having at least 85%, 90%, 91%, 92%, 93%, 94%, 95%, 96%, 97%, 98%, 99%, or 100% identity to any one of SEQ ID NOs: 3887-3912. In some embodiments, the BCMA-directed CAR is encoded by a nucleic acid comprising a sequence having at least 85%, 90%, 91%, 92%, 93%, 94%, 95%, 96%, 97%, 98%, 99%, or 100% identity to any one of SEQ ID NOs: 3913-3964.

In some embodiments, the BCMA-directed CAR is encoded by a nucleic acid that also encodes mbIL15 and has at least 85%, 90%, 91%, 92%, 93%, 94%, 95%, 96%, 97%, 98%, 99%, or 100% identity to any one of SEQ ID NOs: 3991-4042.

In some embodiments, the BCMA-directed CAR comprises a sequence having at least 85%, 90%, 91%, 92%, 93%, 94%, 95%, 96%, 97%, 98%, 99%, or 100% identity to any one of SEQ ID NOs: 4043-4068. In some embodiments, the BCMA-directed CAR is encoded by a nucleic acid comprising a sequence having at least 85%, 90%, 91%, 92%, 93%, 94%, 95%, 96%, 97%, 98%, 99%, or 100% identity to any one of SEQ ID NOs: 4069-4120. In some embodiments, the BCMA-directed CAR is encoded by a nucleic acid that also encodes mbIL15 and has at least 85%, 90%, 91%, 92%, 93%, 94%, 95%, 96%, 97%, 98%, 99%, or 100% identity to any one of SEQ ID NOs: 4147-4198.

In some embodiments, the BCMA-directed CAR comprises a sequence having at least 85%, 90%, 91%, 92%, 93%, 94%, 95%, 96%, 97%, 98%, 99%, or 100% identity to any one of SEQ ID NOs: 4199-4224. In some embodiments, the BCMA-directed CAR is encoded by a nucleic acid comprising a sequence having at least 85%, 90%, 91%, 92%, 93%, 94%, 95%, 96%, 97%, 98%, 99%, or 100% identity to any one of SEQ ID NOs: 4225-4276. In some embodiments, the BCMA-directed CAR is encoded by a nucleic acid that also encodes mbIL15 and has at least 85%, 90%, 91%, 92%, 93%, 94%, 95%, 96%, 97%, 98%, 99%, or 100% identity to any one of SEQ ID NOs: 4303-4354.

In some embodiments, the BCMA-directed CAR comprises a sequence having at least 85%, 90%, 91%, 92%, 93%, 94%, 95%, 96%, 97%, 98%, 99%, or 100% identity to any one of SEQ ID NOs: 4355-4380. In some embodiments, the BCMA-directed CAR is encoded by a nucleic acid comprising a sequence having at least 85%, 90%, 91%, 92%, 93%, 94%, 95%, 96%, 97%, 98%, 99%, or 100% identity to any one of SEQ ID NOs: 4381-4432. In some embodiments, the BCMA-directed CAR is encoded by a nucleic acid that also encodes mbIL15 and has at least 85%, 90%, 91%, 92%, 93%, 94%, 95%, 96%, 97%, 98%, 99%, or 100% identity to any one of SEQ ID NOs: 4459-4510.

In some embodiments, the BCMA-directed CAR comprises a sequence having at least 85%, 90%, 91%, 92%, 93%, 94%, 95%, 96%, 97%, 98%, 99%, or 100% identity to any one of SEQ ID NOs: 4513-4531. In some embodiments, the BCMA-directed CAR is encoded by a nucleic acid comprising a sequence having at least 85%, 90%, 91%, 92%, 93%, 94%, 95%, 96%, 97%, 98%, 99%, or 100% identity to any one of SEQ ID NOs: 4531-4569. In some embodiments, the BCMA-directed CAR is encoded by a nucleic acid that also encodes mbIL15 and has at least 85%, 90%, 91%, 92%, 93%, 94%, 95%, 96%, 97%, 98%, 99%, or 100% identity to any one of SEQ ID NOs: 4589-4626.

In some embodiments, the BCMA-directed CAR comprises a sequence having at least 85%, 90%, 91%, 92%, 93%, 94%, 95%, 96%, 97%, 98%, 99%, or 100% identity to any one of SEQ ID NOs: 4627-4645. In some embodiments, the BCMA-directed CAR is encoded by a nucleic acid comprising a sequence having at least 85%, 90%, 91%, 92%, 93%, 94%, 95%, 96%, 97%, 98%, 99%, or 100% identity to any one of SEQ ID NOs: 4646-4683. In some embodiments, the BCMA-directed CAR is encoded by a nucleic acid that also encodes mbIL15 and has at least 85%, 90%, 91%, 92%, 93%, 94%, 95%, 96%, 97%, 98%, 99%, or 100% identity to any one of SEQ ID NOs: 4703-4740.

In some embodiments, the BCMA-directed CAR comprises a sequence having at least 85%, 90%, 91%, 92%, 93%, 94%, 95%, 96%, 97%, 98%, 99%, or 100% identity to any one of SEQ ID NOs: 4741-4759. In some embodiments, the BCMA-directed CAR is encoded by a nucleic acid comprising a sequence having at least 85%, 90%, 91%, 92%, 93%, 94%, 95%, 96%, 97%, 98%, 99%, or 100% identity to any one of SEQ ID NOs: 4760-4797. In some embodiments, the BCMA-directed CAR is encoded by a nucleic acid that also encodes mbIL15 and has at least 85%, 90%, 91%, 92%, 93%, 94%, 95%, 96%, 97%, 98%, 99%, or 100% identity to any one of SEQ ID NOs: 4817-4854.

In some embodiments, the BCMA-directed CAR comprises a sequence having at least 85%, 90%, 91%, 92%, 93%, 94%, 95%, 96%, 97%, 98%, 99%, or 100% identity to any one of SEQ ID NOs: 4855-4873. In some embodiments, the BCMA-directed CAR is encoded by a nucleic acid comprising a sequence having at least 85%, 90%, 91%, 92%, 93%, 94%, 95%, 96%, 97%, 98%, 99%, or 100% identity to any one of SEQ ID NOs: 4874-4911. In some embodiments, the BCMA-directed CAR is encoded by a nucleic acid that also encodes mbIL15 and has at least 85%, 90%, 91%, 92%, 93%, 94%, 95%, 96%, 97%, 98%, 99%, or 100% identity to any one of SEQ ID NOs: 4931-4968.

In some embodiments, the BCMA-directed CAR comprises a sequence having at least 85%, 90%, 91%, 92%, 93%, 94%, 95%, 96%, 97%, 98%, 99%, or 100% identity to any one of SEQ ID NOs: 4969-4994. In some embodiments, the BCMA-directed CAR is encoded by a nucleic acid comprising a sequence having at least 85%, 90%, 91%, 92%, 93%, 94%, 95%, 96%, 97%, 98%, 99%, or 100% identity to any one of SEQ ID NOs: 4995-5046. In some embodiments, the BCMA-directed CAR is encoded by a nucleic acid that also encodes mbIL15 and has at least 85%, 90%, 91%, 92%, 93%, 94%, 95%, 96%, 97%, 98%, 99%, or 100% identity to any one of SEQ ID NOs: 5073-5124.

In some embodiments, the BCMA-directed CAR comprises a sequence having at least 85%, 90%, 91%, 92%, 93%, 94%, 95%, 96%, 97%, 98%, 99%, or 100% identity to any one of SEQ ID NOs: 5125-5150. In some embodiments, the BCMA-directed CAR is encoded by a nucleic acid comprising a sequence having at least 85%, 90%, 91%, 92%, 93%, 94%, 95%, 96%, 97%, 98%, 99%, or 100% identity to any one of SEQ ID NOs: 5151-5176. In some embodiments, the BCMA-directed CAR is encoded by a nucleic acid that also encodes mbIL15 and has at least 85%, 90%, 91%, 92%, 93%, 94%, 95%, 96%, 97%, 98%, 99%, or 100% identity to any one of SEQ ID NOs: 5203-5228.

In some embodiments, the BCMA-directed CAR comprises a sequence having at least 85%, 90%, 91%, 92%, 93%, 94%, 95%, 96%, 97%, 98%, 99%, or 100% identity to any one of SEQ ID NOs: 5229-5247. In some embodiments, the BCMA-directed CAR is encoded by a nucleic acid comprising a sequence having at least 85%, 90%, 91%, 92%, 93%, 94%, 95%, 96%, 97%, 98%, 99%, or 100% identity to any one of SEQ ID NOs: 5248-5285. In some embodiments, the BCMA-directed CAR is encoded by a nucleic acid that also encodes mbIL15 and has at least 85%, 90%, 91%, 92%, 93%, 94%, 95%, 96%, 97%, 98%, 99%, or 100% identity to any one of SEQ ID NOs: 5305-5342.

In some embodiments, the BCMA-directed CAR comprises a sequence having at least 85%, 90%, 91%, 92%, 93%, 94%, 95%, 96%, 97%, 98%, 99%, or 100% identity to any one of SEQ ID NOs: 5343-5361. In some embodiments, the BCMA-directed CAR is encoded by a nucleic acid comprising a sequence having at least 85%, 90%, 91%, 92%, 93%, 94%, 95%, 96%, 97%, 98%, 99%, or 100% identity to any one of SEQ ID NOs: 5362-5399. In some embodiments, the BCMA-directed CAR is encoded by a nucleic acid that also encodes mbIL15 and has at least 85%, 90%, 91%, 92%, 93%, 94%, 95%, 96%, 97%, 98%, 99%, or 100% identity to any one of SEQ ID NOs: 5419-5456.

In some embodiments, the BCMA-directed CAR comprises a sequence having at least 85%, 90%, 91%, 92%, 93%, 94%, 95%, 96%, 97%, 98%, 99%, or 100% identity to any one of SEQ ID NOs: 5503-5525. In some embodiments, the BCMA-directed CAR is encoded by a nucleic acid comprising a sequence having at least 85%, 90%, 91%, 92%, 93%, 94%, 95%, 96%, 97%, 98%, 99%, or 100% identity to any one of SEQ ID NOs: 5526-5571. In some embodiments, the BCMA-directed CAR is encoded by a nucleic acid that also encodes mbIL15 and has at least 85%, 90%, 91%, 92%, 93%, 94%, 95%, 96%, 97%, 98%, 99%, or 100% identity to any one of SEQ ID NOs: 5595-5640.

In some embodiments, the BCMA-directed CAR comprises a sequence having at least 85%, 90%, 91%, 92%, 93%, 94%, 95%, 96%, 97%, 98%, 99%, or 100% identity to any one of SEQ ID NOs: 5641-5663. In some embodiments, the BCMA-directed CAR is encoded by a nucleic acid comprising a sequence having at least 85%, 90%, 91%, 92%, 93%, 94%, 95%, 96%, 97%, 98%, 99%, or 100% identity to any one of SEQ ID NOs: 5664-5709. In some embodiments, the BCMA-directed CAR is encoded by a nucleic acid that also encodes mbIL15 and has at least 85%, 90%, 91%, 92%, 93%, 94%, 95%, 96%, 97%, 98%, 99%, or 100% identity to any one of SEQ ID NOs: 5733-5778.

In some embodiments, the BCMA-directed CAR comprises a VHH domain and comprises a sequence having at least 85%, 90%, 91%, 92%, 93%, 94%, 95%, 96%, 97%, 98%, 99%, or 100% identity to any one of SEQ ID NOs: 5779-5801. In some embodiments, the BCMA-directed VHH-CAR is encoded by a nucleic acid comprising a sequence having at least 85%, 90%, 91%, 92%, 93%, 94%, 95%, 96%, 97%, 98%, 99%, or 100% identity to any one of SEQ ID NOs: 5802-5847. In some embodiments, the BCMA-directed VHH-CAR is encoded by a nucleic acid that also encodes mbIL15 and has at least 85%, 90%, 91%, 92%, 93%, 94%, 95%, 96%, 97%, 98%, 99%, or 100% identity to any one of SEQ ID NOs: 5871-5916.

It shall be appreciated that, for any receptor construct described herein, certain sequence variability, extensions, and/or truncations of the disclosed sequences may result when combining sequences, as a result of, for example, ease or efficiency in cloning (e.g., for creation of a restriction site).

### Methods of Treatment and Administration and Dosing

Disclosed herein are populations of immune cells comprising any one of the anti-BCMA binding moieties, BCMA-directed CARs, or BCMA-directed CAR constructs disclosed herein. In some embodiments, the immune cells are NK cells and/or T cells. In some embodiments, the population of immune cells further comprise an additional extracellular moiety or CAR that binds a non-BCMA cancer marker. In some embodiments, the non-BCMA cancer marker comprises one or more of CD138, SLAMF7, CD38, GPRC5D, or CD19.

Also provided herein are embodiments relating to methods of treating, ameliorating, inhibiting, or preventing cancer with an immune cell or population of immune cells comprising any one of the anti-BCMA binding moieties, BCMA-directed CARs, or BCMA-directed CAR constructs disclosed herein. In some embodiments, the methods comprise administering a therapeutically effective amount of the immune cell or population of immune cells comprising any one of the anti-BCMA binding moieties, BCMA-directed CARs, or BCMA-directed CAR constructs disclosed herein.

In certain embodiments, treatment of a subject with a genetically engineered cell(s) described herein achieves one, two, three, four, or more of the following effects, including, for example: (i) reduction or amelioration the severity of disease or symptom associated therewith; (ii) reduction in the duration of a symptom associated with a disease; (iii) protection against the progression of a disease or symptom associated therewith; (iv) regression of a disease or symptom associated therewith; (v) protection against the development or onset of a symptom associated with a disease; (vi) protection against the recurrence of a symptom associated with a disease; (vii) reduction in the hospitalization of a subject; (viii) reduction in the hospitalization length; (ix) an increase in the survival of a subject with a disease; (x) a reduction in the number of symptoms associated with a disease; (xi) an enhancement, improvement, supplementation, complementation, or augmentation of the prophylactic or therapeutic effect(s) of another therapy. Each of these comparisons are versus, for example, a different therapy for a disease, which includes a cell-based immunotherapy for a disease using cells that do not express the constructs disclosed herein.

Administration can be by a variety of routes, including, without limitation, intravenous, intra-arterial, subcutaneous, intramuscular, intrahepatic, intraperitoneal and/or local delivery to an affected tissue. Doses of immune cells such as NK and/or T cells can be readily determined for a given subject based on their body mass, disease type and state, and desired aggressiveness of treatment, but range, depending on the embodiments, from about 10⁵ cells per kg to about 10¹² cells per kg (e.g., 10⁵-10⁷, 10⁷-10¹⁰, 10¹⁰-10¹² and overlapping ranges therein). In one embodiment, a dose escalation regimen is used. In several embodiments, a range of immune cells such as NK and/or T cells is administered, for example between about 1×10⁶ cells/kg to about 1×10⁸ cells/kg. Depending on the embodiment, various types of cancer can be treated. In several embodiments, hepatocellular carcinoma is treated. Additional embodiments provided for herein include treatment or prevention of the following non-limiting examples of cancers including, but not limited to, acute lymphoblastic leukemia (ALL), acute myeloid leukemia (AML), adrenocortical carcinoma, Kaposi sarcoma, lymphoma, gastrointestinal cancer, appendix cancer, central nervous system cancer, basal cell carcinoma, bile duct cancer, bladder cancer, bone cancer, brain tumors (including but not limited to astrocytomas, spinal cord tumors, brain stem glioma, glioblastoma, craniopharyngioma, ependymoblastoma, ependymoma, medulloblastoma, medulloepithelioma), breast cancer, bronchial tumors, Burkitt lymphoma, cervical cancer, colon cancer, chronic lymphocytic leukemia (CLL), chronic myelogenous leukemia (CML), chronic myeloproliferative disorders, ductal carcinoma, endometrial cancer, esophageal cancer, gastric cancer, Hodgkin lymphoma, non-Hodgkin lymphoma, hairy cell leukemia, renal cell cancer, leukemia, oral cancer, nasopharyngeal cancer, liver cancer, lung cancer (including but not limited to, non-small cell lung cancer, (NSCLC) and small cell lung cancer), pancreatic cancer, bowel cancer, lymphoma, melanoma, ocular cancer, ovarian cancer, pancreatic cancer, prostate cancer, pituitary cancer, uterine cancer, and vaginal cancer.

Also provided are uses of engineered NK cell as disclosed herein for the treatment of cancer and/or for the preparation of a medicament for the treatment of cancer. In several embodiments, the cancer is multiple myeloma.

In several embodiments, polynucleotides encoding the disclosed chimeric antigen receptors (including, but not limited to BCMA-directed chimeric antigen receptors) are mRNA. In some embodiments, the polynucleotide is DNA. In some embodiments, the polynucleotide is operably linked to at least one regulatory element for the expression of the cytotoxic receptor complex.

Additionally provided, according to several embodiments, is a vector comprising the polynucleotide encoding any of the polynucleotides provided for herein, wherein the polynucleotides are optionally operatively linked to at least one regulatory element for expression of a cytotoxic receptor complex. In several embodiments, the vector is a retrovirus.

Further provided herein are engineered immune cells (such as NK and/or T cells) comprising the polynucleotide, vector, or cytotoxic receptor complexes as disclosed herein. Further provided herein are compositions comprising a mixture of engineered immune cells (such as NK cells and/or engineered T cells), each population comprising the polynucleotide, vector, or cytotoxic receptor complexes as disclosed herein.

### Cancer Types

Some embodiments of the compositions and methods described herein relate to administering immune cells comprising a chimeric antigen receptor, such as a BCMA-directed chimeric antigen receptor, to a subject with cancer. Various embodiments provided for herein include treatment or prevention of the following non-limiting examples of cancers. Examples of cancer include, but are not limited to, multiple myeloma, acute lymphoblastic leukemia (ALL), acute myeloid leukemia (AML), adrenocortical carcinoma, Kaposi sarcoma, lymphoma, gastrointestinal cancer, appendix cancer, central nervous system cancer, basal cell carcinoma, bile duct cancer, bladder cancer, bone cancer, brain tumors (including but not limited to astrocytomas, spinal cord tumors, brain stem glioma, craniopharyngioma, ependymoblastoma, ependymoma, medulloblastoma, medulloepithelioma), breast cancer, bronchial tumors, Burkitt lymphoma, cervical cancer, colon cancer, chronic lymphocytic leukemia (CLL), chronic myelogenous leukemia (CML), chronic myeloproliferative disorders, ductal carcinoma, endometrial cancer, esophageal cancer, gastric cancer, Hodgkin lymphoma, non-Hodgkin lymphoma, hairy cell leukemia, renal cell cancer, leukemia, oral cancer, nasopharyngeal cancer, liver cancer, lung cancer (including but not limited to, non-small cell lung cancer, (NSCLC) and small cell lung cancer), pancreatic cancer, bowel cancer, lymphoma, melanoma, ocular cancer, ovarian cancer, pancreatic cancer, prostate cancer, pituitary cancer, uterine cancer, and vaginal cancer.

### Cancer Targets

Some embodiments of the compositions and methods described herein relate to immune cells comprising one or more chimeric antigen receptors that target a cancer antigen. Non-limiting examples of target antigens include: BCMA, CD19, CD38, CD138 (also known as syndecan 1), G protein-coupled receptor, class C group 5 member D (GPRC5D), SLAMF7, CD229 (SLAMF3), CD123, DLL3, epidermal growth factor receptor (EGFR), prostate-specific membrane antigen (PSMA), Fms Like Tyrosine Kinase 3 (FLT3); KREMEN2 (Kringle Containing Transmembrane Protein 2, Alkaline phosphatase, placental-like 2 (ALPPL2, claudin 4, claudin 6, CD5, CD22; CD30; CD171; CS1 (also referred to as CD2 subset 1, CRACC, SLAMF7, CD319, and 19A24); C-type lectin-like molecule-1 (CLL-1 or CLECLI); CD33; epidermal growth factor receptor variant III (EGFRviii); ganglioside G2 (GD2); ganglioside GD3 (aNeu5Ac(2-8)aNeu5Ac(2-3)bDGalp(1-4)bDGlcp(1-1)Cer); Tn antigen ((Tn Ag) or (GalNAca-Ser/Thr)); prostate-specific membrane antigen (PSMA); Receptor tyrosine kinase-like orphan receptor 1 (ROR1); Fms Like Tyrosine Kinase 3 (FLT3); Tumor-associated glycoprotein 72 (TAG72); CD38; CD44v6; a glycosylated CD43 epitope expressed on acute leukemia or lymphoma but not on hematopoietic progenitors, a glycosylated CD43 epitope expressed on non-hematopoietic cancers, Carcinoembryonic antigen (CEA); Epithelial cell adhesion molecule (EPCAM); B7H3 (CD276); KIT (CD117); Interleukin-13 receptor subunit alpha-2 (IL-13Ra2 or CD213A2); Mesothelin; Interleukin 11 receptor alpha (IL-11Ra); prostate stem cell antigen (PSCA); Protease Serine 21 (Testisin or PRSS21); vascular endothelial growth factor receptor 2 (VEGFR2); Lewis(Y) antigen; CD24; Platelet-derived growth factor receptor beta (PDGFR-beta); Stage-specific embryonic antigen-4 (SSEA-4); CD20; Folate receptor alpha (FRa or FR1); Folate receptor beta (FRb); Receptor tyrosine-protein kinase ERBB2 (Her2/neu); Mucin 1, cell surface associated (MUC1); epidermal growth factor receptor (EGFR); neural cell adhesion molecule (NCAM); Prostase; prostatic acid phosphatase (PAP); elongation factor 2 mutated (ELF2M); Ephrin B2; fibroblast activation protein alpha (FAP); insulin-like growth factor 1 receptor (IGF-I receptor), carbonic anhydrase IX (CAIX); Proteasome (Prosome, Macropain) Subunit, Beta Type, 9 (LMP2); glycoprotein 100 (gp100); oncogene fusion protein consisting of breakpoint cluster region (BCR) and Abelson murine leukemia viral oncogene homolog 1 (Abl) (bcr-abl); tyrosinase; ephrin type-A receptor 2 (EphA2); sialyl Lewis adhesion molecule (sLe); ganglioside GM3 (aNeu5Ac(2-3)bDClalp(1-4)bDGlcp(1-1)Cer); transglutaminase 5 (TGS5); high molecular weight-melanoma associated antigen (HMWMAA); o-acetyl-GD2 ganglioside (OAcGD2); tumor endothelial marker 1 (TEM1/CD248); tumor endothelial marker 7-related (TEM7R); claudin 6 (CLDN6); thyroid stimulating hormone receptor (TSHR); G protein coupled receptor class C group 5, member D (GPRC5D); chromosome X open reading frame 61 (CXORF61); CD97; CD179a; anaplastic lymphoma kinase (ALK); Polysialic acid; placenta-specific 1 (PLAC1); hexasaccharide portion of globoH glycoceramide (GloboH); mammary gland differentiation antigen (NY-BR-1); uroplakin 2 (UPK2); Hepatitis A virus cellular receptor 1 (HAVCR1); adrenoceptor beta 3 (ADRB3); pannexin 3 (PANX3); G protein-coupled receptor 20 (GPR20); lymphocyte antigen 6 complex, locus K 9 (LY6K); Olfactory receptor 51E2 (OR51E2); TCR Gamma Alternate Reading Frame Protein (TARP); Wilms tumor protein (WT1); Cancer/testis antigen 1 (NY-ESO-1); Cancer/testis antigen 2 (LAGE-la); Melanoma-associated antigen 1 (MAGE-A1); ETS translocation-variant gene 6, located on chromosome 12p (ETV6-AML); sperm protein 17 (SPA17); X Antigen Family, Member 1A (XAGE1); angiopoietin-binding cell surface receptor 2 (Tie 2); melanoma cancer testis antigen-1 (MAD-CT-1); melanoma cancer testis antigen-2 (MAD-CT-2); Fos-related antigen 1; tumor protein p53 (p53); p53 mutant; prostein; survivin; telomerase; prostate carcinoma tumor antigen-1 (PCT A-1 or Galectin 8), melanoma antigen recognized by T cells 1 (MelanA or MARTI); Rat sarcoma (Ras) mutant; human Telomerase; reverse transcriptase (hTERT); sarcoma translocation breakpoints; melanoma inhibitor of apoptosis (ML-IAP); ERG (transmembrane protease, serine 2 (TMPRSS2) ETS fusion gene); N-Acetyl glucosaminyl-transferase V (NA17); paired box protein Pax-3 (PAX3); Androgen receptor; Cyclin B1; v-myc avian myelocytomatosis viral oncogene neuroblastoma derived homolog (MYCN); Ras Homolog Family Member C (RhoC); Tyrosinase-related protein 2 (TRP-2); Cytochrome P450 IB 1 (CYPIB 1); CCCTC-Binding Factor (Zinc Finger Protein)-Like (BORIS or Brother of the Regulator ofimprinted Sites), Squamous Cell Carcinoma Antigen Recognized By T Cells 3 (SART3); Paired box protein Pax-5 (PAX5); proacrosin binding protein sp32 (OY-TES1); lymphocyte-specific protein tyrosine kinase (LCK); A kinase anchor protein 4 (AKAP-4); synovial sarcoma, X breakpoint 2 (SSX2); Receptor for Advanced Gly cation Endproducts (RAGE-1); renal ubiquitous 1 (RU1); renal ubiquitous 2 (RU2); legumain; human papilloma virus E6 (HPV E6); human papilloma virus E7 (HPV E7); intestinal carboxyl esterase; heat shock protein 70-2 mutated (mut hsp70-2); CD79a; CD79b; CD72; Leukocyte-associated immunoglobulin-like receptor 1 (LAIR1); Fc fragment of IgA receptor (FCAR or CD89); Leukocyte immunoglobulin-like receptor subfamily A member 2 (LILRA2); CD300 molecule-like family member f (CD300LF); C-type lectin domain family 12 member A (CLEC12A); bone marrow stromal cell antigen 2 (BST2); EGF-like module-containing mucin-like hormone receptor-like 2 (EMR2); lymphocyte antigen 75 (LY75); Glypican-3 (GPC3); Fc receptor-like 5 (FCRL5); and immunoglobulin lambda-like polypeptide 1 (IGLLI), MPL, Biotin, c-MYC epitope Tag, CD34, LAMP1 TROP2, GFRalpha4, CDH17, CDH6, NYBR1, CDH19, CD200R, Slea (CA19.9; Sialyl Lewis Antigen); Fucosyl-GMI, PTK7, gpNMB, CDH1-CD324, DLL3, CD276/B7H3, IL1 1Ra, IL13Ra2, CD179b-IGLll, TCRgamma-delta, NKG2D, CD32 (FCGR2A), Tn ag, Timl-/HVCR1, CSF2RA (GM-CSFR-alpha), TGFbetaR2, Lews Ag, TCR-beta1 chain, TCR-beta2 chain, TCR-gamma chain, TCR-delta chain, FITC, Leutenizing hormone receptor (LHR), Follicle stimulating hormone receptor (FSHR), Gonadotropin Hormone receptor (CGHR or GR), CCR4, GD3, SLAMF6, SLAMF4, HIV1 envelope glycoprotein, HTLVI-Tax, CMV pp65, EBV-EBNA3c, KSHV K8.1, KSHV-gH, influenza A hemagglutinin (HA), GAD, PDL1, Guanylyl cyclase C (GCC), auto antibody to desmoglein 3 (Dsg3), auto antibody to desmoglein 1 (Dsgl), HLA, HLA-A, HLA-A2, HLA-B, HLA-C, HLA-DP, HLA-DM, HLA-DOA, HLA-DOB, HLA-DQ, HLA-DR, HLA-G, IgE, CD99, Ras G12V, Tissue Factor 1 (TF1), AFP, GPRC5D, Claudin1 8.2 (CLD18A2 or CLDN18A.2)), P-glycoprotein, STEAP1, Liv1, Nectin-4, Cripto, gpA33, BST1/CD157, low conductance chloride channel, and the antigen recognized by TNT antibody.

### EXAMPLES

The materials and methods disclosed herein are non-limiting examples that are to be employed according to certain embodiments disclosed herein.

### Example 1—Anti-BCMA CAR Expression in NK Cells

According to several embodiments, NK cells will be isolated from peripheral blood mononuclear cells and expanded through the use of a feeder cell line. In several embodiments, the feeder cells are engineered to express certain stimulatory molecules (e.g. interleukins, CD3, 4-1BBL, etc.) to promote immune cell expansion and activation. Engineered feeder cells and related methods for expanding NK cells are disclosed in, for example, U.S. Pat. No. 7,435,596 or 8,026,097, International Patent Application PCT/SG2018/050138, International Patent Application PCT/US2020/044033, and U.S. Provisional Patent Application No. 63/073,671, each of which is expressly incorporated by reference in its entirety herein.

NK cells isolated from PBMC will be co-cultured with K562 cells expressing membrane-bound IL15 and 4-1BBL, with the media to be supplemented with IL2. Viral transduction, with a vector encoding an anti-BCMA-directed chimeric antigen receptor construct, such as any one of those disclosed herein, will be performed at approximately Day 7. Various anti-BCMA CAR constructs will be transduced into different populations of NK cells. Any combination of one or more transmembrane domains, one or more hinge domains, one or more co-stimulatory domains and one or more signaling domains disclosed herein may be used. In several embodiments, the anti-BCMA CAR will comprise an OX40 domain and a CD3ζ signaling domain. In several embodiments, the viral vector will also encode interleukin 15, optionally in a membrane-bound format to be expressed by the NK cells along with the anti-BCMA CAR. The resultant engineered NK cells will be evaluated at 14, or more, days of total culture time.

Expression of the anti-BCMA CAR constructs will be evaluated by detecting the CAR construct, for example by assessing the percentage of NK cells in a test population that express a tag sequence integrated into the CAR (e.g., a FLAG epitope tag or the CD34 and CD20 epitopes of the RQRCD8 hinge), although embodiments disclosed herein also provide for epitope tag-free CAR constructs. It is believed that at least about 75% or more of the NK cells will express the CAR in a stable manner (e.g., for at least 2-3 weeks in culture or more).

This is a prophetic example.

### Example 2—Anti-BCMA CAR Expression in T Cells

According to several embodiments, T cells will be isolated from peripheral blood mononuclear cells and expanded through the use of commercially available T cell expansion products (e.g., beads coupled to anti-CD3 and anti-CD28 antibodies).

Viral transduction of the T cells, with various vector encoding an anti-BCMA-directed chimeric antigen receptor construct, such as any one of those disclosed herein, will be performed at approximately Day 7. Various anti-BCMA CAR constructs will be transduced into different populations of T cells. Any combination of one or more transmembrane domains, one or more hinge domains, one or more co-stimulatory domains and one or more signaling domains disclosed herein may be used. In several embodiments, the anti-BCMA CAR will comprise an OX40 domain and a CD3ζ signaling domain. In several embodiments, the viral vector will also encode interleukin 15, optionally in a membrane-bound format to be expressed by the T cells along with the anti-BCMA CAR. The resultant engineered T cells will be evaluated at 14, or more, days of total culture time.

Expression of the anti-BCMA CAR constructs will be evaluated by detecting the CAR construct, for example by assessing the percentage of T cells in a test population that express a tag sequence integrated into the CAR (e.g., a FLAG epitope tag or the CD34 and CD20 epitopes of the RQRCD8 hinge), although embodiments disclosed herein also provide for epitope tag-free CAR constructs. It is believed that at least about 75% or more of the T cells will express the CAR in a stable manner (e.g., for at least 2-3 weeks in culture or more).

This is a prophetic example.

### Example 3—In Vitro Assessment of Cytotoxicity of Anti-BCMA CAR-Expressing NK and T Cells

NK cells and/or T cells expressing various anti-BCMA CARs, such as those disclosed herein, will be co-cultured with tumor cells expressing BCMA as well as cells expressing little or no BCMA as a control. Non-transduced NK and/or T cells can also be used as a negative control. Tumor cells will optionally be tagged with a fluorescent detection tag (e.g., GFP) for detection/quantification by flow cytometry. Various effector:target (E:T) ratios will be assessed, for example 8:1, 4:1, 2:1, 1:1, 1:2, 1:4, and/or 1:8. After co-culture, culture media will be collected and assayed for levels of various cytotoxic or proinflammatory cytokines. Tumor cell survival will be quantified.

It is believed that NK and/or T cells expressing anti-BCMA CARs and co-cultured with tumor cells expressing BCMA will result in higher release of cytotoxic effector molecules (such as Granzyme B, perforin, and/or interferon gamma) as compared to release of those effectors by non-transduced NK and/or T cells and BCMA-CAR-expressing NK and/or T cells cultured with tumor cells expressing reduced BCMA levels.

It is believed that NK and/or T cells expressing anti-BCMA CARs and co-cultured with tumor cells expressing BCMA will exhibit cytotoxic effects against the tumor cells in a manner dependent on the E:T ratio of a given experiment. It is believed that the engineered NK and/or T cells will exhibit anti-tumor cell effects that are persistent in nature (e.g., able to exhibit cytotoxicity for at least 2-3 weeks post transduction with the anti-BCMA CAR.

This is a prophetic example.

### Example 4—In Vivo Assessment of Cytotoxicity of Anti-BCMA CAR-Expressing NK and T Cells

NK and T cells will be isolated from PBMCs and expanded as described herein. NK cells and T cells will be engineered to express anti-BCMA CARs, such as any one of those disclosed herein, through viral transduction of the NK or T cells. Viral transduction will be performed at approximately Day 7 post-isolation. Various anti-BCMA CAR constructs will be transduced into different populations of NK cells and T cells. Any combination of one or more transmembrane domains, one or more hinge domains, one or more co-stimulatory domains and one or more signaling domains disclosed herein may be used. In several embodiments, the anti-BCMA CAR will comprise an OX40 domain and a CD3ζ signaling domain. In several embodiments, the viral vector will also encode interleukin 15, optionally in a membrane-bound format to be expressed by the NK cells along with the anti-BCMA CAR. The resultant engineered NK and engineered T cells will be evaluated at 14, or more, days of total culture time.

Immunodeficient NSG mice will be injected intravenously on Day 0 with BCMA-positive tumor cells (e.g., multiple myeloma cells such as NCI-H929, U266-B1, or RPMI-8226) at an appropriate dose (e.g., 1×10⁵ cells) and expressing a luminescence marker. At Day 1, mice will receive either a PBS control injection, non-transduced NK and/or T cells, or NK and/or T cells expressing one of the various anti-BCMA CARs disclosed herein. Bioluminescent imaging data will be collected at various time points, such as Day 0, Day 8, Day 11, Day 16, Day 20, Day 28, Day 32, and Day 40. Blood samples will be collected at various time points, such as Day 5, Day 15 and Day 20, Day 25, Day 30 Day, 35, and Day 40.

Blood samples will be analyzed for the presence and number of tumor cells using flow cytometry to detect BCMA or another identifying cell surface protein. Bioluminescent images will be reviewed for signal intensity across time points. It is expected that bioluminescence signal will increase over time for the PBS-control group and the non-transduced NK and/or T cells, indicating expansion of the tumor cells. It is anticipated that injection of NK cells expressing an anti-BCMA CAR will result in reduced progression of tumor cell growth. It is anticipated that injection of T cells expressing an anti-BCMA CAR will result in reduced progression of tumor cell growth. It is anticipated that injection of a combination of NK cells expressing an anti-BCMA CAR and T cells expressing an anti-BCMA CAR will yield marked, or even synergistic, reduction in the progression of tumor cell growth. In embodiments in which an additional epitope of BCMA is targeted (e.g., by a bi-specific CAR or a second CAR expressed by the NK and/or T cells), further enhanced cytotoxicity is expected.

It is expected that mice receiving NK cells and/or T cells expressing an anti-BCMA CAR (or CARs) will exhibit enhanced survival rate over the control groups.

This is a prophetic example.

### Example 5—Combinations of Other Cancer Targets with Anti-BCMA CAR-Expressing NK and/or T Cells

NK and T cells will be isolated from PBMCs and expanded as described herein. NK cells and T cells will be engineered to express anti-BCMA CARs, such as any one of those disclosed herein, through viral transduction of the NK or T cells. Viral transduction will be performed at approximately Day 7 post-isolation. Various anti-BCMA CAR constructs will be transduced into different populations of NK cells and T cells. Any combination of one or more transmembrane domains, one or more hinge domains, one or more co-stimulatory domains and one or more signaling domains disclosed herein may be used. In several embodiments, the anti-BCMA CAR will comprise an OX40 domain and a CD3ζ signaling domain. In several embodiments, the viral vector will also encode interleukin 15, optionally in a membrane-bound format to be expressed by the NK cells along with the anti-BCMA CAR.

NK cells and/or T cells will be engineered to express a CAR directed against an additional, e.g., non-BCMA, tumor marker. The additional tumor marker will be one or more of CD19, CD38, CD138, SLAM-F7, or GPRC5D, or other tumor marker generally known in the art. In several embodiments, a single CAR is engineered to target both BCMA and one or more of CD19, CD38, CD138, SLAM-F7, or GPRC5D, or other tumor marker generally known in the art. As with the BCMA-targeting CARs, any combination of one or more transmembrane domains, one or more hinge domains, one or more co-stimulatory domains and one or more signaling domains disclosed herein may be used. In several embodiments, the CAR directed against a non-BCMA marker will comprise an OX40 domain and a CD3ζ signaling domain. In several embodiments, the viral vector used to transduce NK and/or T cells with the non-BCMA CAR will also encode interleukin 15, optionally in a membrane-bound format to be expressed by the NK cells along with the non-BCMA CAR (or bispecific CAR). The resultant engineered NK and engineered T cells will be evaluated at 14, or more, days of total culture time.

Immunodeficient NSG mice will be injected intravenously on Day 0 with tumor cells (at an appropriate dose, such as 1×10⁵ cells) that are BCMA-positive, positive for one or more non-BCMA tumor markers (e.g., CD19, CD38, CD138, SLAM-F7, or GPRC5D) and expressing a luminescence marker. At Day 1, mice will receive either a PBS control injection, non-transduced NK and/or T cells, or NK and/or T cells expressing one of the various anti-BCMA CARs disclosed herein, expressing one of the various non-BCMA CARs disclosed herein, or a bispecific BCMA/non-BCMA CAR. Bioluminescent imaging data will be collected at various time points, such as Day 0, Day 8, Day 11, Day 16, Day 20, Day 28, Day 32, and Day 40. Blood samples will be collected at various time points, such as Day 5, Day 15 and Day 20, Day 25, Day 30 Day, 35, and Day 40.

Blood samples will be analyzed for the presence and number of tumor cells using flow cytometry to detect BCMA and the non-BCMA cell surface protein. Bioluminescent images will be reviewed for signal intensity across time points. It is expected that bioluminescence signal will increase over time for the PBS-control group and the non-transduced NK and/or T cells, indicating expansion of the tumor cells. It is anticipated that injection of NK cells, T cells, and/or combinations of NK cells with T cells expressing an anti-BCMA CAR and a CAR directed to a non-BCMA target will result in reduced progression of tumor cell growth. It is anticipated that this reduction will yield tumor growth reductions that are greater than those achieved by cells expressing either an anti-BCMA or non-BCMA CAR would yield alone. In several embodiments, similar reductions in tumor cell growth will be expected with a bi-specific CAR targeting BCMA and a non-BCMA target, whether expressed on NK cells, T cells, or on both NK cell and T cells in combination.

It is expected that mice receiving NK cells and/or T cells expressing an anti-BCMA CAR and a non-BCMA targeting CAR (or a single, bi-specific CAR) will exhibit enhanced survival rate over the control groups as well as over groups treated with cells expressing only one CAR (either BCMA or non-BCMA-directed).

This is a prophetic example.

### Example 6—Screen of Tonic Versus Target Binding-Induced Signaling

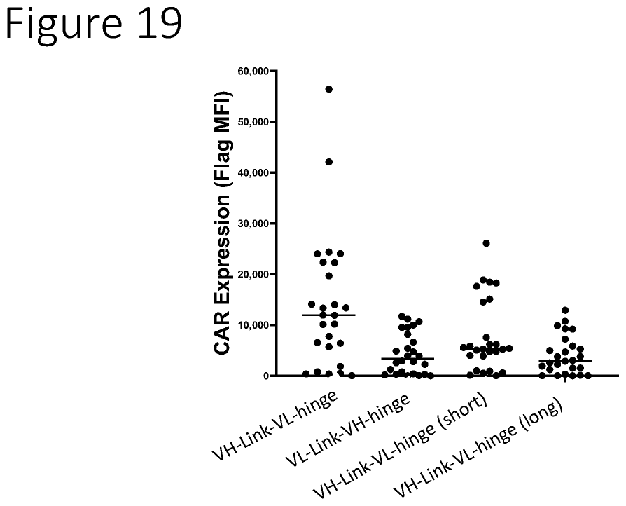

As described above, CARs provided for herein employ various structural components and/or use the same component in a different structural configuration. In order to select CARs for use in immunotherapy, a series of experiments was conducted to evaluate how the various CAR components and CAR formats disclosed herein affect expression, target binding, and target binding-induced activation as compared to tonic signaling (e.g., signaling from the CAR in the absence of the target). As depicted in the Figures (as non-limiting embodiments) and as described herein, CARs employ various structures depending on the embodiment. For example, in several embodiments different hinge domains are used to operably connect the tumor binding portion of the CAR to the remainder of the CAR (e.g., the transmembrane and signaling regions). In several embodiments, CD8 alpha-derived domains are used. For example, in several embodiments, the CD8 hinge, transmembrane and intracellular domains are used in a CAR. In several embodiments, CD28 is used. In several embodiments, longer or shorter hinge domains are used. Likewise, for scFv-containing CARs may employ a VH-linker-VL or VL-linker-VH format, noting that the linker may be different between constructs. FIG. 19 shows the results of generating four non-limiting CAR structures, a VH-linker-VL-CD8 alpha hinge, a VL-linker-VH-CD8 alpha hinge, a VH-linker-VL-IgG4 hinge (short), and a VH-linker-VL-RQRCD8 hinge (long). Expression was measured by detection of a FLAG tag embedded in the nucleotide sequence encoding the construct. It shall be appreciated that any constructs presented herein that employ a FLAG (or other detection tag) are also envisioned absent the tag (and any corresponding additional sequences, such as an associated linker). Expression data for FIG. 19 are shown as Mean Fluorescence Intensity (MFI).

As shown in FIG. 19, each of these formats, regardless of linker employed were expressed by Jurkat cells. While expression did not appear to be significantly impacted by the choice of linker in this experiment, the VH-linker-VL scFv format did appear to result in greater expression levels. However, in several embodiments, the VL-linker-VH scFv format is still able to expressed sufficiently well to enable CARs employing such scFvs to bind the respective target (e.g., BCMA) and induce cytotoxicity against such a target-expressing tumor cell.

Cancer immunotherapy requires that engineered cells are able to bind tumor cells and largely or completely avoid targeting and acting on non-tumor cells. Certain CARs are responsive primarily, or only, when they bind the tumor marker to which they have been engineered to recognize. This is referred to herein as activation. Other CARs, which are less desirable for use in immunotherapy, exhibit signaling even when their corresponding tumor marker is not present. This is referred to herein as tonic signaling. Certain experiments discussed herein relate to the ratio of activation to tonic signaling, in which a larger value for the ratio represents greater signaling for a given CAR and/or less tonic signaling.

Activation and tonic signaling are evaluated in a model system for assessing signaling in which Jurkat cells are used (as a surrogate for other cells to be used in cell therapy). Jurkat cells are an immortal human leukemic T cell line that have seen widespread use in the assessment of T cell activation and signaling mechanisms. Although Jurkat cells do not secrete the complete repertoire cytokines that primary T cells do, and Jurkat cells lack significant cytolytic activity, Jurkat cells do produce IL-2 and upregulate CD69 upon activation. Due to the ease with which cell-surface CD69 expression can be detected using fluorescent antibodies (e.g., through flow-cytometry), evaluation of the induced expression of CD69 staining was used to determine activation of the Jurkat cells expressing CARs provided for herein in the presence and absence of BCMA-expressing target cells (here MM.S1 cells).

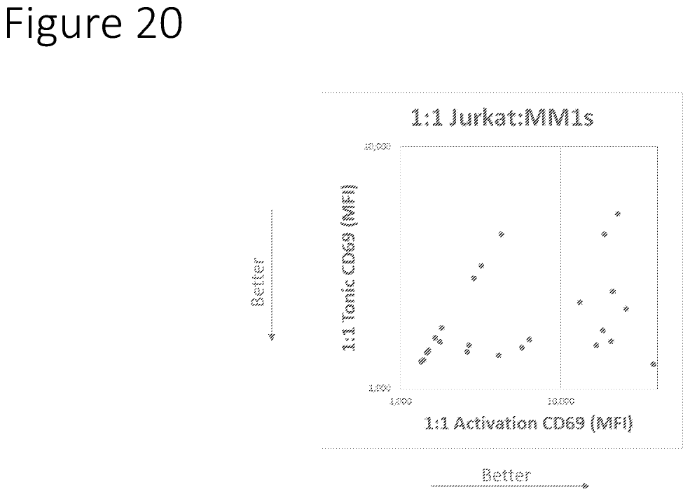

FIG. 20 shows the resultant data from an E:T ratio of 1:1 (Jurkat:MM.1S) which is plotted based off signaling of the engineered Jurkat cells in the absence of target cells. As compared to similar experiments when smaller numbers of tumor cells were present (e.g., E:T of 10:1, data not shown), an increase in the number of target tumor cells results in an increase in the expression of CD69. As shown in the scatterplot, several of the clones exhibit high activation as well as elevated tonic signaling (upper right). Likewise, some clones show low activation and elevated tonic signaling or low activation and low signaling (left portion of scatterplot). Select clones, however, show high activation and low tonic signaling, making them attractive for potential use in cancer immunotherapy.

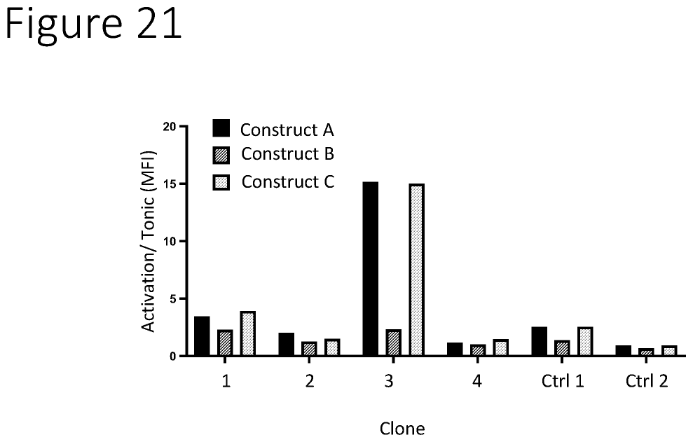

The data such as those related to the tonic signaling and BCMA binding can be used to generate a ratio of BCMA binding-induced signaling to tonic signaling, which can serve as a data point to compare a given CAR to others in terms of on-target performance. FIG. 21 shows selected non-limiting CAR constructs that were compared in terms of their activation/tonic signaling ratio. Each of Construct A, Construct B, and Construct C have different architecture (e.g., order of VH/VL domains, linker, etc. Within a given architecture, each of Clone 1, 2, 3, and 4 vary in terms of their binder sequence. Two different control CAR architectures were used for control. The sequence of tumor marker binder within a given CAR architecture does afford some differences in terms of the activation/tonic signaling ratio. For example, Clone A1 appears to have an elevated ratio as compared to Clone A4. Similar results are seen, for example with Clone C1 as compared to C4. Marked differences are shown in comparing Clone A1 or C1 as compared to Clone A3 or C3. While the overall Clone 1 and Clone 3 groups share a similar pattern, Clone A3 and C3 show markedly higher activation/tonic signaling ratios. These data demonstrate that the architecture of a CAR can significantly impact the activation/tonic signaling ratio. Thus, according to several embodiments, CAR architecture (e.g., VH-linker A-VL, VL-linker A-VH, VH-linker B-VL, or VL-linker B-VH) can be optimized to enhance the degree of activity that is due to binding of the CAR to a tumor marker, such as BCMA, while reducing/eliminating tonic signaling that occurs even when the tumor marker is minimally expressed, or not expressed at all.

### Example 7—Assessment of CAR Components on Expression and Function

Building on the results of the prior Example, further experiments were undertaken to evaluate BCMA-directed CAR expression and function. As provided for herein, two series of CARs were designed and constructed that varied in their architecture, as outlined below.

TABLE 1
CAR Architectures

| | | | | | | | | | |
|---|---|---|---|---|---|---|---|---|---|
| Structure | Binder Clone | Chain 1 | Linker | Chain 2 | Hinge | TM | IC | Co-stim | Stim |

Round 1

| | | | | | | | | | |
|---|---|---|---|---|---|---|---|---|---|
| A | 1-26 | VH | GS3 | VL | CD8 | CD8 | CD8 | OX40 | CD3z |
| B | 1-26 | VL | | VH | CD8 | | | | |
| C | 1-26 | VH | | VL | Short | | | | |
| D | 1-26 | VH | | VL | Long | | | | |
| E.1 | 1^{st} of 26 | VL | Whitlow | VH | CD8 | | | | |
| E.2 | 2^{nd} of 26 | VL | | VH | CD8 | | | | |
| E.3 | 3^{rd} of 26 | VL | | VH | CD8 | | | | |
| E.4 | 4^{th} of 26 | VL | | VH | CD8 | | | | |

Round 2

| | | | | | | | | | |
|---|---|---|---|---|---|---|---|---|---|
| F | 1-19 | VH | GS3 | VL | CD8 | CD8 | CD8 | Ox40 | CD3z |
| G | 1-19 | VH | Whitlow | VL | | | | | |
| H | 1-19 | VL | | VH | | | | | |
| I | 1-19 | VH | GS3 | VL | CD28 | CD28 | CD28 | | |

Based on the various structures above, assays were performed to determine if particular CAR architecture, and particular sequences within a given architecture, result in enhanced expression and/or activity (e.g., elevated activation with reduced tonic signaling).

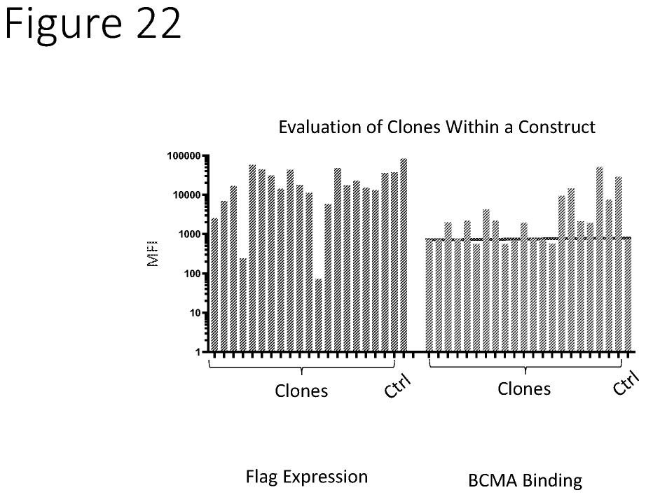

FIG. 22 show a series of histograms that relate to expression of each of the clones within a given CAR architecture (architecture F is shown in this Figure as non-limiting data). The left panel of FIG. 22 shows expression of CARs by Jurkat cells as measured by detection of a FLAG tag integrated into the nucleic acid encoding the CAR. Data are shown as MFI, which represents the degree to which a particular cell expresses the Car (e.g., “copies” of the CAR per cell). As can be seen, even within a given architecture, certain clone sequences express more robustly than others. The right panel of FIG. 22 relates to the ability of each of the clones within a given architecture to bind BCMA, the intended target. Interestingly, while some robustly expressing cells achieved elevated BCMA binding, even some of those that were low expressing still exhibited BCMA binding on par with other CARs that had higher mean expression. This data indicate that not only is sequence important for expression, but that binding can be independent of expression. Analysis of both aspects of a CAR is thus important, in several embodiments, in so far as a lower-expressing CAR may overcome that lower expression level with elevated binding activity.

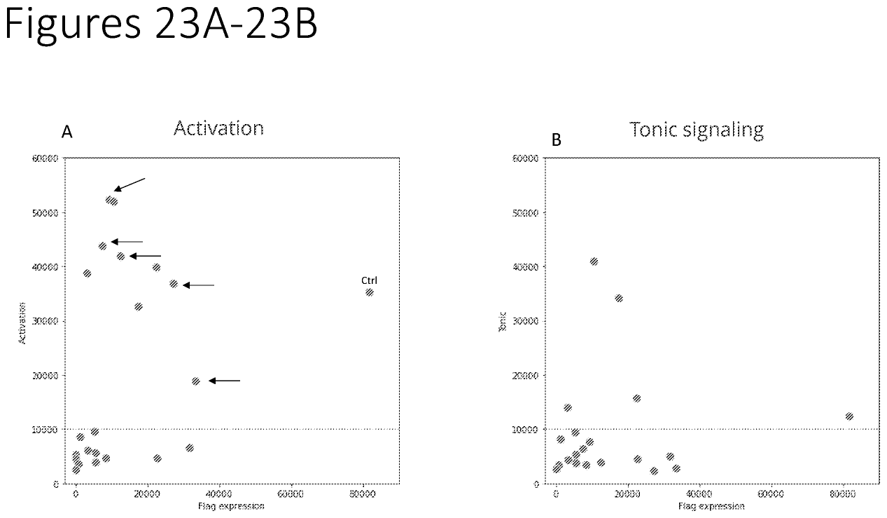

Another characteristic that can be evaluated, as discussed above, is the activation (based on target binding) to tonic signaling ratio. This ratio helps to evaluate the potential “on-” and “off-target” potential for a particular CAR. FIGS. 23A-23B shows scatter plots related to BCMA binding-induced activation (23A) and undesired tonic signaling (23B). As can be seen in FIG. 23A, several of the constructs (from varying architectures and also within an architecture) achieve activation of the CAR based on binding to BCMA. These are depicted as the circles above the 10,000 value threshold horizontal line. Those below the threshold line were deemed to have insufficient activation for passing through the screen (at least based on activation alone). FIG. 23B shows the corresponding data for tonic signaling. Again those CAR-expressing cells that are activated, even in the absence of all or substantially all target tumor marker, are deemed to exhibit some degree of tonic signaling. Those circles above the 10,000 unit threshold are deemed to show some degree of tonic signaling. Those that are below the threshold do not. However, assessing either tonic signaling or activation alone is a partial analysis. In looking at the clones that show activation above the threshold, but do not exhibit tonic signaling, five CARs satisfy those criteria in this particular screen. They are identified by arrows in FIG. 23A.

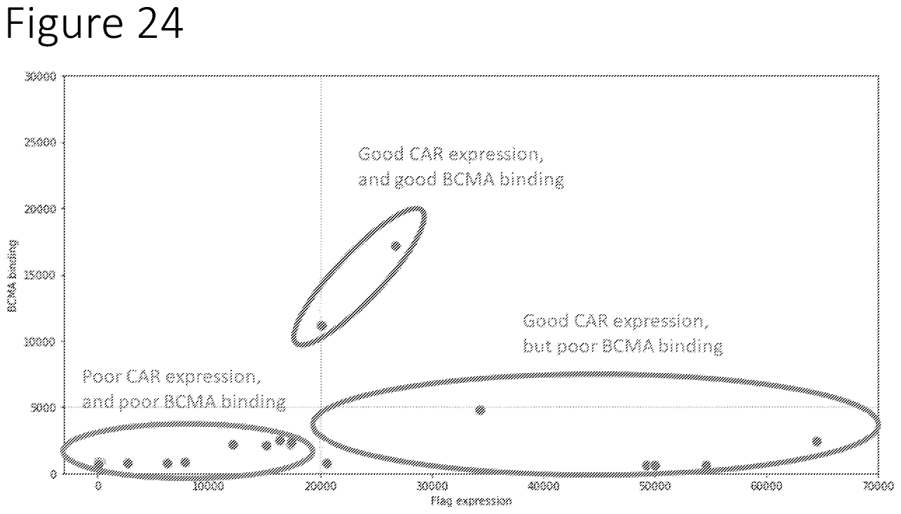

Building on this analysis, FIG. 24 shows a scatterplot of BCMA binding as a function of CAR expression, with labels indicating various groupings of the CARs. The lower left of the plot shows those CARS that neither express sufficiently well nor show sufficient BCMA binding. The lower right shows the subpopulation of CARs that express well, but show insufficient BCMA binding. The two clones in the central portion of the plot are those that exhibit good expression and good BCMA binding. However, as discussed above, for a more robust understanding of a given CAR, expression and activation can be considered along with an assessment of tonic signaling (or lack thereof).

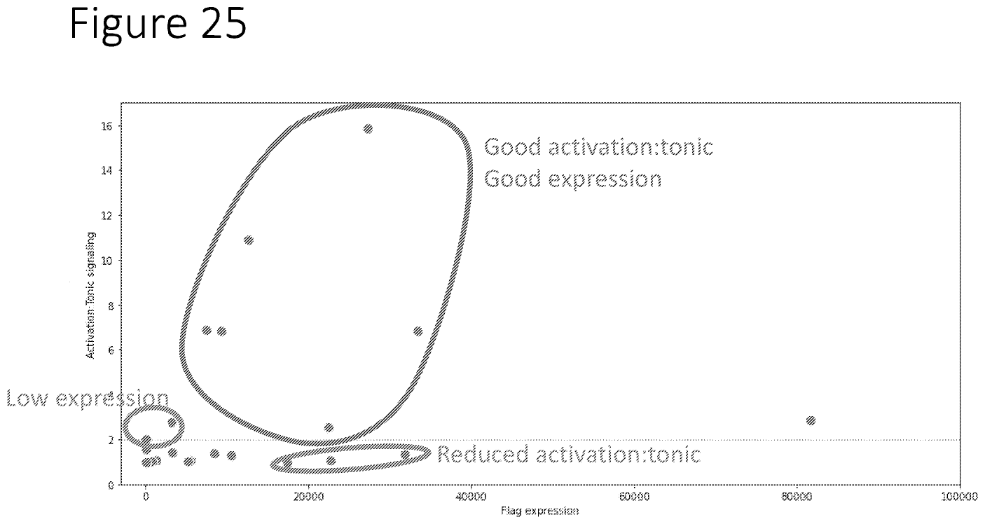

FIG. 25 shows a scatterplot of the ratio of activation/tonic signaling in a screen of a portion of the CARs provided for herein. The activation/tonic signaling ratio is shown on the Y axis, with expression shown on the X axis. As with the prior figure, those in the lower left portion show low expression or a low activation/tonic signaling ratio (indicative of a greater than desired degree of tonic signaling, which lowers the ratio). The lower right shows those clones with reasonable expression, but undesirable activation/tonic signaling ratios. The large number of clones in the central portion of the graph exhibit varied amounts of acceptable expression levels and higher activation/tonic signaling ratios.

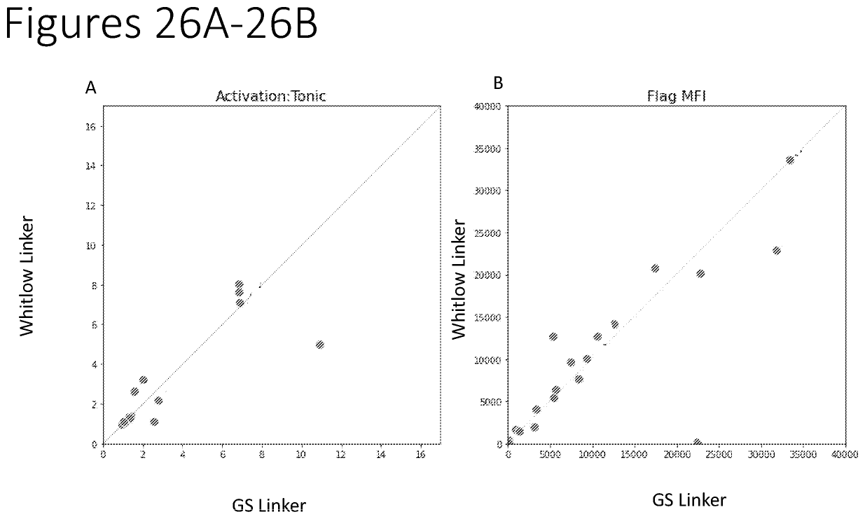

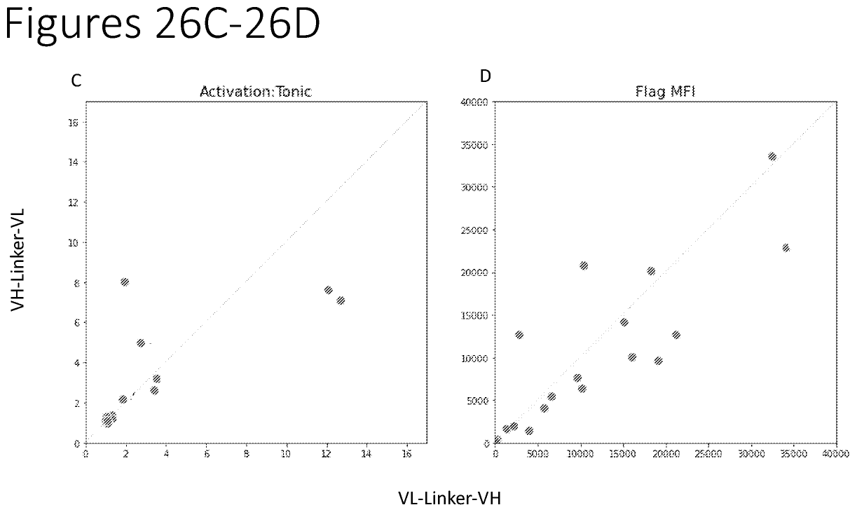

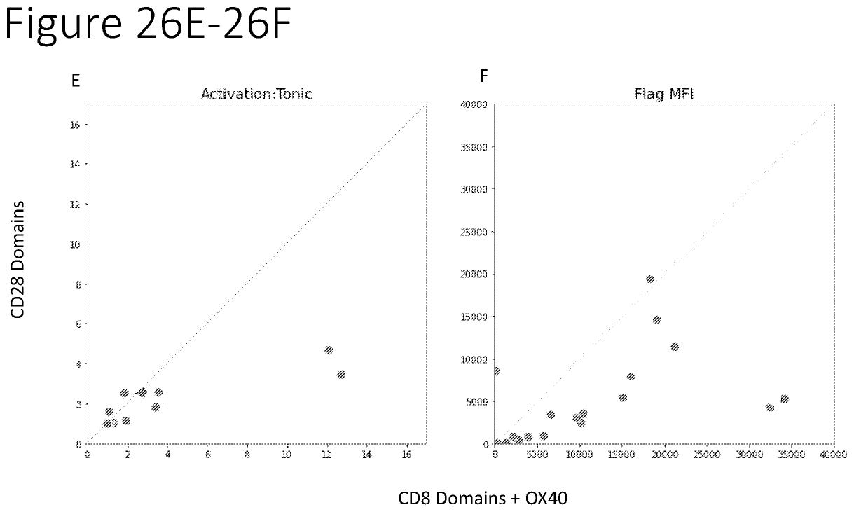

Further assessment of the impact of CAR architecture was undertaken to determine if a particular feature of CAR architecture showed a dominant impact on expression, target binding, or activation/tonic signaling. FIGS. 26A and 26B show data related to the activation/tonic signaling ratio (26A) and expression (26B) when using a Whitlow versus a GS linker. While certain individual clones appeared to behave uniquely, the majority of clones did not appear to deviate from the norm in terms of activation/tonic signaling or expression. A somewhat greater distribution of expression and activation/tonic signaling ratio was identified when comparing the orientation of heavy and light chains in the scFv. FIG. 26C shows the activation/tonic signaling ratio when comparing VH-linker-VL scFv structure (Y axis) versus VL-linker-VH scFv structure (X axis). While certain clones show improved activation with VH-VL ordering, others show better activity with VL-VH ordering. Similar, clone-specific, results are shown in FIG. 26D with respect to expression.

Because certain CARs in this screen differed in the sub-components of the transmembrane and signaling domains, an additional comparison was made to compare the activation/tonic signaling ratio in those CARs with CD28-derived sequences for the hinge, transmembrane and intracellular domains as compared to those using corresponding domains from CD8 alpha (with the OX40 co-stimulatory domain. Here, the data as shown in FIG. 26E seem to suggest a general trend towards improved activation/tonic signaling ratio when the CD8-OX40 domains are used, as opposed to the CD28 domains. This also appears to be the case, perhaps even more clearly, with respect to expression of the CAR constructs, as shown in FIG. 26F. These data show that both structural and sequence-based changes can influence CAR expression, binding, and activation. According to several embodiments disclosed herein, BCMA-directed CARs can achieve suitable expression in immune cells (such as NK cells), and desirable levels of activation and tonic signaling, making them appropriate candidates for BCMA-directed cancer immunotherapy.

### Example 8—Evaluation of VHH-Containing CARs

Also provided for herein are CARs that, rather than an scFv tumor binder format, employ a camelid-based tumor binder. In several embodiments, a monovalent (single VHH) CAR is used, while in other embodiments a bivalent (VHH-linker-VHH) CAR format is used. Similar to the examples discussed above, experiments were undertaken to determine if structural or sequence-based alterations to the VHH-CAR architecture impacted expression, target binding, and/or activation of the cell expressing the VHH-CAR. Monovalent or bivalent VHH-CARs were expressed in Jurkat and assessed for expression, BCMA binding, and activation/tonic signaling ratio.

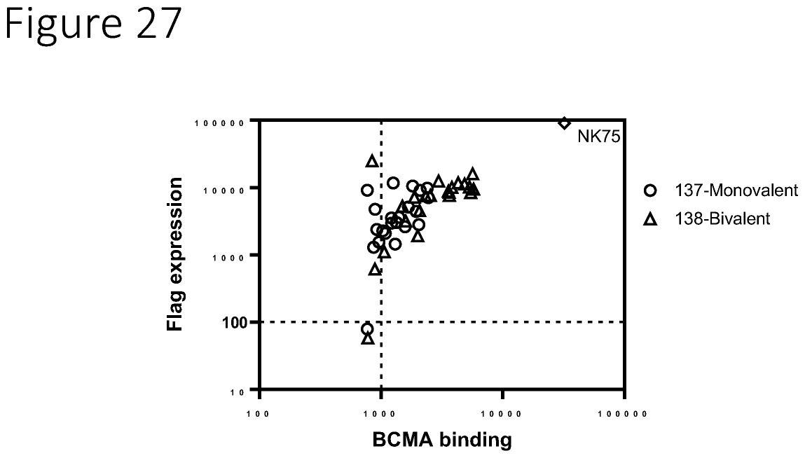

FIG. 27 shows data plotting the degree of expression of monovalent and bivalent VHH-CARs relative to BCMA binding. These data indicate a relatively similar degree of expression for both monovalent (circles) and bivalent (triangles) VHH-CARs, albeit with variation among the individual clones. These data also appear to show a trend for bivalent VHH-CARs to show increased BMCA binding. This is not entirely unexpected, given the dual binding domain format of a bivalent VHH-CAR.

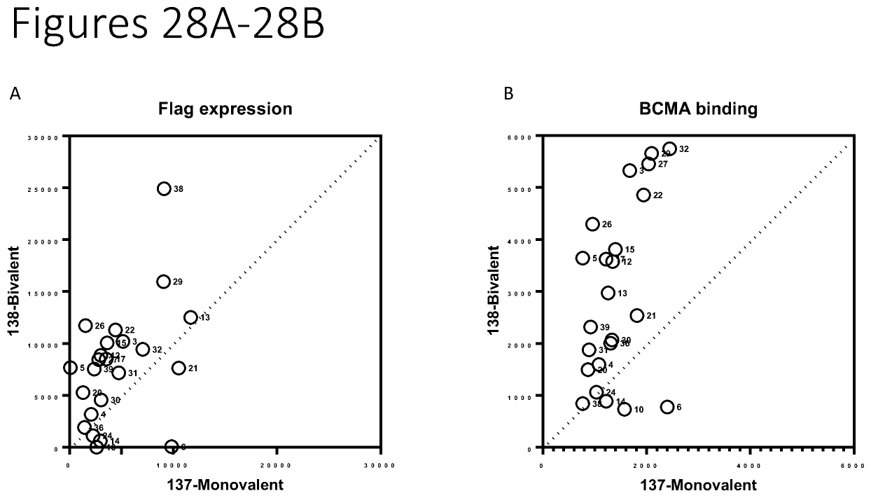

FIGS. 28A-28B further support this initial suggestion. FIG. 28A shows data for expression of bivalent versus monovalent VHH-CARs. As shown in the scatterplot, most of the data points lie above the threshold line of equivalence, indicating that expression is greater for the bivalent VHH-CARs. Similar, if not more profound, results are shown in FIG. 28B, where BCMA binding appears more robust when using bivalent VHH-CARs as compared to monovalent VHH-CARs.

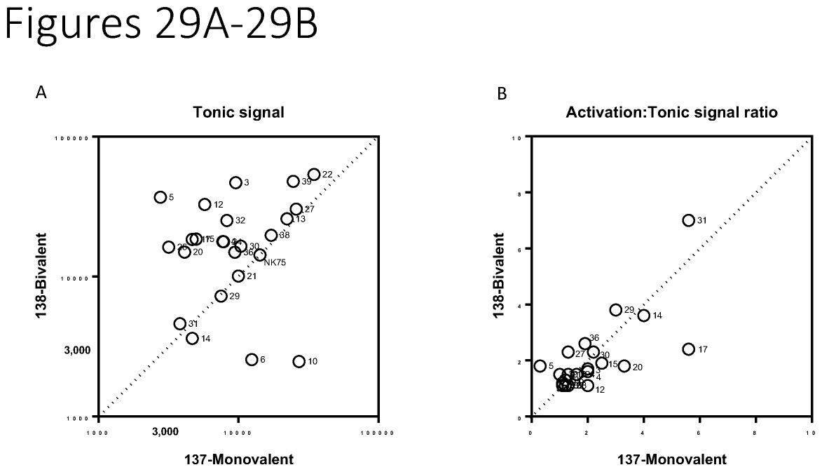

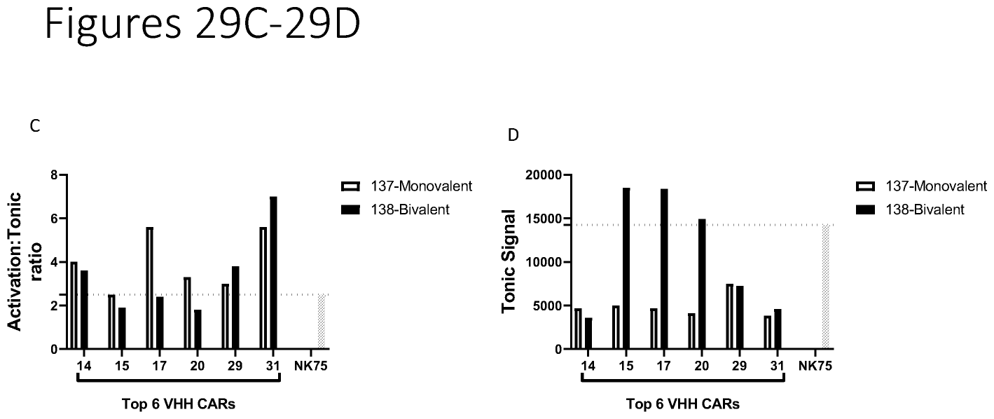

As discussed above, however, there is a need to understand the potential interplay between binding of a target tumor marker and the activation of a cell expressing a CAR in the absence of a target tumor marker (e.g., tonic activation). FIG. 29A shows data related to the detection of tonic signaling for bivalent and monovalent VHH-CARs. These data suggest that tonic signaling is more prevalent in a bivalent VHH-CAR format. FIG. 29B assesses the activation/tonic signaling ratio, which shows a fairly even distribution of the ratio among the monovalent versus bivalent format. This may be explained, in part, by the apparent increase in BMCA binding shown by bivalent constructs. Thus, both the numerator (binding/activation) and the denominator (tonic signaling) are increased in the bivalent format, thus making the evaluation based on the ratio appear more similar to that of the monovalent.

FIGS. 29C-29D break out a subset of the data, using the six VHH-CARs showing the most preferred characteristics for the monovalent and bivalent formats. FIG. 29C is a histogram comparing the activation/tonic signaling ratio. These data suggest that the monovalent format shows a generally higher ratio than the bivalent format. The underlying mechanism appears to be borne out in FIG. 29B, which shows that the tonic signaling of the bivalent format is much greater than the monovalent construct.

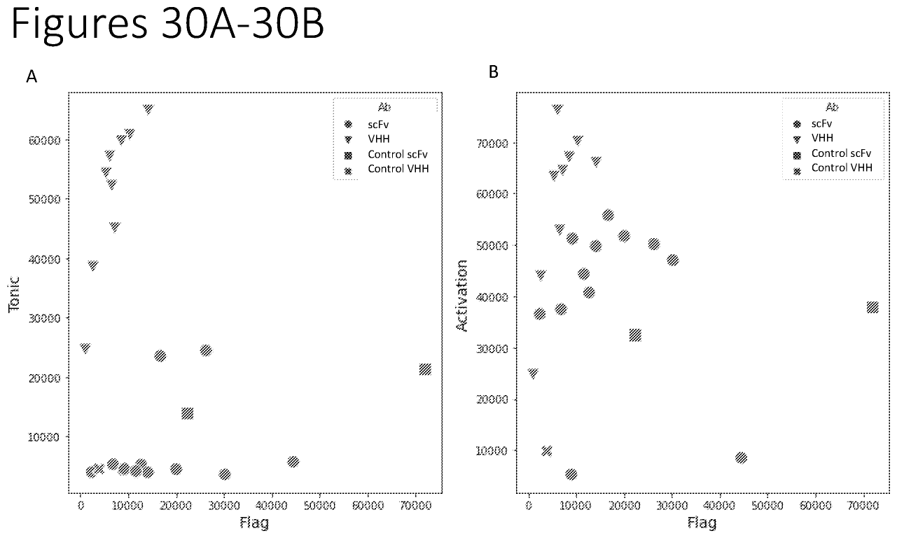

Taking each of these examples into account, an analysis was performed to compare scFv-CARs with VHH-CARs. These data are shown in FIGS. 30A and 30B. FIG. 30A shows the degree of tonic signaling as a function of expression. Candidate VHH-CARs show significantly greater tonic signaling than candidate scFv's (as well as control VHH-CARs). FIG. 30B shows data related to cell activation and reveal that, while VHH-CARs in general have higher activation than scFv, the nominally lower activation when scFv CARs bind BCMA, in conjunction with the dramatically lower tonic signaling, scFv CAR formats appear to perform in a preferred manner, making them therefore likely candidates for use in cellular immunotherapy. While this particular data suggests that scFv-CARs are preferred, in some embodiments/formats, VHH-CARs may be utilized as well. Nonetheless, these data suggest that an independent evaluation of each of these parameters may be warranted to fully understand the features of a given CAR.

It is contemplated that various combinations or subcombinations of the specific features and aspects of the embodiments disclosed above may be made and still fall within one or more of the inventions. Further, the disclosure herein of any particular feature, aspect, method, property, characteristic, quality, attribute, element, or the like in connection with an embodiment can be used in all other embodiments set forth herein. Accordingly, it should be understood that various features and aspects of the disclosed embodiments can be combined with or substituted for one another in order to form varying modes of the disclosed inventions. Thus, it is intended that the scope of the present inventions herein disclosed should not be limited by the particular disclosed embodiments described above. Moreover, while the invention is susceptible to various modifications, and alternative forms, specific examples thereof have been shown in the drawings and are herein described in detail. It should be understood, however, that the invention is not to be limited to the particular forms or methods disclosed, but to the contrary, the invention is to cover all modifications, equivalents, and alternatives falling within the spirit and scope of the various embodiments described and the appended claims. Any methods disclosed herein need not be performed in the order recited. The methods disclosed herein include certain actions taken by a practitioner; however, they can also include any third-party instruction of those actions, either expressly or by implication. In addition, where features or aspects of the disclosure are described in terms of Markush groups, those skilled in the art will recognize that the disclosure is also thereby described in terms of any individual member or subgroup of members of the Markush group.

The ranges disclosed herein also encompass any and all overlap, sub-ranges, and combinations thereof. Language such as “up to,” “at least,” “greater than,” “less than,” “between,” and the like includes the number recited. Numbers preceded by a term such as “about” or “approximately” include the recited numbers. For example, “about 90%” includes “90%.” In some embodiments, a sequence having at least 95% sequence identity includes sequences having 96%, 97%, 98%, 99%, and 100% sequence identity to the reference sequence. In addition, when a sequence is disclosed as “comprising” a nucleotide or amino acid sequence, such a reference shall also include, unless otherwise indicated, that the sequence “comprises”, “consists of” or “consists essentially of” the recited sequence.

In several embodiments, there are provided amino acid sequences that correspond to any of the nucleic acids disclosed herein, while accounting for degeneracy of the nucleic acid code. Furthermore, those sequences (whether nucleic acid or amino acid) that vary from those expressly disclosed herein, but have functional similarity or equivalency are also contemplated within the scope of the present disclosure. The foregoing includes mutants, truncations, substitutions, or other types of modifications.

Any titles or subheadings used herein are for organization purposes and should not be used to limit the scope of embodiments disclosed herein.

All references cited herein, including but not limited to published and unpublished applications, patents, and literature references, are incorporated herein by reference in their entirety and are hereby made a part of this specification. To the extent publications and patents or patent applications incorporated by reference contradict the disclosure contained in the specification, the specification is intended to supersede and/or take precedence over any such contradictory material.

1-87. (canceled)

88. An anti-BCMA binding moiety comprising a heavy chain variable region (VH) comprising a HCDR1, a HCDR2, and a HCDR3, and a light chain variable region (VL) comprising a LCDR1, a LCDR2, and a LCDR3, wherein the VH comprises the HCDR1, the HCDR2, and the HCDR3 contained within the VH amino acid sequence set forth in any one of SEQ ID NOS:260-285 and 1582-1600; and the VL comprises the LCDR1, the LCDR2, and the LCDR3 contained within the VL amino acid sequence set forth in any one of SEQ ID NOS:541-566 and 1677-1695.

89. The anti-BCMA binding moiety of claim 88, wherein the VH comprises an amino acid sequence having at least 90% sequence identity to the amino acid sequence set forth in any one of SEQ ID NOS:260-285 and 1582-1600, and the VL comprises an amino acid sequence having at least 90% sequence identity to the amino acid sequence set forth in any one of SEQ ID NOS:541-566 and 1677-1695.

90. The anti-BCMA binding moiety of claim 88, wherein the VH comprises the amino acid sequence set forth in any one of SEQ ID NOS:260-285 and 1582-1600, and the VL comprises the amino acid sequence set forth in any one of SEQ ID NOS:541-566 and 1677-1695.

91. The anti-BCMA binding moiety of claim 88, wherein:
(a) the VH comprises the HCDR1, the HCDR2, and the HCDR3 contained within the VH amino acid sequence set forth in SEQ ID NO:268, and the VL comprises the LCDR1, the LCDR2, and the LCDR3 contained within the VL amino acid sequence set forth in SEQ ID NO:549;
(b) the VH comprises the HCDR1, the HCDR2, and the HCDR3 contained within the VH amino acid sequence set forth in SEQ ID NO:270, and the VL comprises the LCDR1, the LCDR2, and the LCDR3 contained within the VL amino acid sequence set forth in SEQ ID NO:551;
(c) the VH comprises the HCDR1, the HCDR2, and the HCDR3 contained within the VH amino acid sequence set forth in SEQ ID NO:1600, and the VL comprises the LCDR1, the LCDR2, and the LCDR3 contained within the VL amino acid sequence set forth in SEQ ID NO:1695; or
(d) the VH comprises the HCDR1, the HCDR2, and the HCDR3 contained within the VH amino acid sequence set forth in SEQ ID NO:1586, and the VL comprises the LCDR1, the LCDR2, and the LCDR3 contained within the VL amino acid sequence set forth in SEQ ID NO:1681.

92. The anti-BCMA binding moiety of claim 88, wherein:
(a) the VH comprises an amino acid sequence having at least 90% sequence identity to the VH amino acid sequence set forth in SEQ ID NO:268, and the VL comprises an amino acid sequence having at least 90% sequence identity to the VL amino acid sequence set forth in SEQ ID NO:549;
(b) the VH comprises an amino acid sequence having at least 90% sequence identity to the VH amino acid sequence set forth in SEQ ID NO:270, and the VL comprises an amino acid sequence having at least 90% sequence identity to the VL amino acid sequence set forth in SEQ ID NO:551;
(c) the VH comprises an amino acid sequence having at least 90% sequence identity to the VH amino acid sequence set forth in SEQ ID NO:1600, and the VL comprises an amino acid sequence having at least 90% sequence identity to the VL amino acid sequence set forth in SEQ ID NO:1695; or
(d) the VH comprises an amino acid sequence having at least 90% sequence identity to the VH amino acid sequence set forth in SEQ ID NO:1586, and the VL comprises an amino acid sequence having at least 90% sequence identity to the VL amino acid sequence set forth in SEQ ID NO:1681.

93. The anti-BCMA binding moiety of claim 88, wherein:
(a) the VH comprises the amino acid sequence set forth in SEQ ID NO:268, and the VL comprises the amino acid sequence set forth in SEQ ID NO:549;
(b) the VH comprises the amino acid sequence set forth in SEQ ID NO:270, and the VL comprises the amino acid sequence set forth in SEQ ID NO:551;
(c) the VH comprises the amino acid sequence set forth in SEQ ID NO:1600, and the VL comprises the amino acid sequence set forth in SEQ ID NO:1695; or
(d) the VH comprises the amino acid sequence set forth in SEQ ID NO:1586, and the VL comprises the amino acid sequence set forth in SEQ ID NO:1681.

94. The anti-BCMA binding moiety of claim 88, wherein the anti-BCMA binding moiety is a single-chain variable fragment (scFv), a Fv, a Fab, or a F(ab′)2.

95. The anti-BCMA binding moiety of claim 88, wherein the VH and VL are joined by a linker comprising the amino acid sequence set forth in SEQ ID NO:1388 or 2388.

96. The anti-BCMA binding moiety of claim 88, wherein the anti-BCMA binding moiety is a single-chain variable fragment (scFv) comprising the amino acid sequence set forth in any one of SEQ ID NOS:593-618 or 671-696.

97. An anti-BCMA binding moiety comprising a first heavy chain variable region (VHH1), wherein the VHH1 comprises a HCDR1, a HCDR2, and a HCDR3 contained within the VHH1 amino acid sequence set forth in any one of SEQ ID NOS:3255-3277.

98. The anti-BCMA binding moiety of claim 97, wherein the VHH1 comprises an amino acid sequence having at least 90% sequence identity to the amino acid sequence set forth in any one of SEQ ID NOS:3255-3277.

99. The anti-BCMA binding moiety of claim 97, wherein:
(a) the VHH1 comprises the HCDR1, the HCDR2, and the HCDR3 contained within the VHH1 amino acid sequence set forth in SEQ ID NO:3262;
(b) the VHH1 comprises the HCDR1, the HCDR2, and the HCDR3 contained within the VHH1 amino acid sequence set forth in SEQ ID NO:3263;
(c) the VHH1 comprises the HCDR1, the HCDR2, and the HCDR3 contained within the VHH1 amino acid sequence set forth in SEQ ID NO:3264;
(d) the VHH1 comprises the HCDR1, the HCDR2, and the HCDR3 contained within the VHH1 amino acid sequence set forth in SEQ ID NO:3265;
(e) the VHH1 comprises the HCDR1, the HCDR2, and the HCDR3 contained within the VHH1 amino acid sequence set forth in SEQ ID NO:3271; or
(f) the VHH1 comprises the HCDR1, the HCDR2, and the HCDR3 contained within the VHH1 amino acid sequence set forth in SEQ ID NO:3273.

100. The anti-BCMA binding moiety of claim 97, further comprising a second heavy chain variable region (VHH2), wherein the VHH2 comprises a HCDR1, a HCDR2, and a HCDR3 contained within the VHH2 amino acid sequence set forth in any one of SEQ ID NOS:3255-3277.

101. The anti-BCMA binding moiety of claim 100, wherein the VHH2 comprises an amino acid sequence having at least 90% sequence identity to the amino acid sequence set forth in any one of SEQ ID NOS:3255-3277.

102. The anti-BCMA binding moiety of claim 100, wherein the anti-BCMA binding moiety comprises the amino acid sequence set forth in any one of SEQ ID NOS:3324-3346.

103. A BCMA-directed chimeric antigen receptor (CAR) comprising:
(a) the anti-BCMA binding moiety of claim 88;
(b) a hinge domain;
(c) a transmembrane domain; and
(d) an intracellular signaling domain comprising a co-stimulatory subdomain and a CD3ζ subdomain.

104. The BCMA-directed CAR of claim 103, wherein the co-stimulatory subdomain comprises an OX40 subdomain, a CD28 subdomain, or a 4-1BB subdomain.

105. A polynucleotide encoding the anti-BCMA binding moiety of claim 88.

106. A polynucleotide encoding the anti-BCMA binding moiety of claim 97.

107. A vector comprising the polynucleotide of claim 105.

108. An immune cell comprising the BCMA-directed CAR of claim 88.

109. The immune cell of claim 108, wherein the immune cell is a natural killer (NK) cell or T cell.

110. A composition comprising a plurality of the immune cells of claim 108.

111. A method for treating a subject having a cancer, the method comprising administering the composition of claim 110 to a subject having a cancer.

112. The method of claim 111, wherein the cancer is multiple myeloma.
